SEQUENCE LISTING (47 sequences)

SEQ ID NO: 1 - RNA, 21 nt, Homo sapiens
uaaagugcugacagugcagau

SEQ ID NO: 2 - RNA, 24 nt, Homo sapiens
aaaagugcuuacagugcagguagc

SEQ ID NO: 3 - RNA, 23 nt, Homo sapiens
uaaagugcuuauagugcagguag

SEQ ID NO: 4 - RNA, 23 nt, Homo sapiens
caaagugcucauagugcagguag

SEQ ID NO: 5 - RNA, 24 nt, Homo sapiens
caaagugcuuacagugcagguagu

SEQ ID NO: 6 - RNA, 24 nt, Homo sapiens
gaaagugcugcgacauuugagcgu

SEQ ID NO: 7 - RNA, 23 nt, Homo sapiens
caaagugcuguucgugcagguag

SEQ ID NO: 8 - RNA, 7 nt, Homo sapiens
aaagugc

SEQ ID NO: 9 - RNA, 21 nt, Homo sapiens
aucugcacugucagcacuuua

SEQ ID NO: 10 - RNA, 24 nt, Homo sapiens
gcuaccugcacuguaagcacuuuu

SEQ ID NO: 11 - RNA, 23 nt, Homo sapiens
cuaccugcacuauaagcacuuua

SEQ ID NO: 12 - RNA, 23 nt, Homo sapiens
cuaccugcacuauaagcacuuug

SEQ ID NO: 13 - RNA, 24 nt, Homo sapiens
acuaccugcacuguaagcacuuug

SEQ ID NO: 14 - RNA, 22 nt, Homo sapiens
cuaccugcacgaacagcacuuu

SEQ ID NO: 15 - RNA, 23 nt, Homo sapiens
cuaccugcacgaacagcacuuug

SEQ ID NO: 16 - RNA, 23 nt, Homo sapiens
acgcucaaaugucgcagcacuuu

SEQ ID NO: 17 - RNA, 24 nt, Homo sapiens
acgcucaaaugucgcagcacuuuc

SEQ ID NO: 18 - RNA, 22 nt, Homo sapiens
aaagugcuguucgugcagguag

SEQ ID NO: 19 - RNA, 23 nt, Homo sapiens
aaagugcugcgacauuugagcgu

SEQ ID NO: 20 - RNA, 21 nt, Homo sapiens
aaagugcuuccuuuuagaggg

SEQ ID NO: 21 - RNA, 21 nt, Homo sapiens
aaagugcuuccuuuuagaggc

SEQ ID NO: 22 - RNA, 23 nt, Homo sapiens
aaagugcauccuuuuagagguuu

SEQ ID NO: 23 - RNA, 22 nt, Homo sapiens
uaaggugcaucuagugcagaua

SEQ ID NO: 24 - RNA, 82 nt, Homo sapiens
ccugccggggcuaaagugcugacagugcagauagugguccucuccgugcuaccgcacuguggguacuugcugcuccagcagg

SEQ ID NO: 25 - RNA, 81 nt, Homo sapiens
ccuuggccauguaaaagugcuuacagugcagguagcuuuuugagaucuacugcaauguaagcacuucuuacauuaccaugg

SEQ ID NO: 26 - RNA, 71 nt, Homo sapiens
guagcacuaaagugcuuauagugcagguaguguuuaguuaucuacugcauuaugagcacuuaaaguacugc

SEQ ID NO: 27 - RNA, 69 nt, Homo sapiens
aguaccaaagugcucauagugcagguaguuuuggcaugacucuacuguaguaugggcacuuccaguacu

SEQ ID NO: 28 - RNA, 84 nt, Homo sapiens
gucagaauaaugucaaagugcuuacagugcagguagugauaugugcaucuacugcagugaaggcacuuguagcauuauggugac

SEQ ID NO: 29 - RNA, 80 nt, Homo sapiens
cugggggcuccaaagugcuguucgugcagguagugugauuacccaaccuacugcugagcuagcacuucccgagcccccgg

SEQ ID NO: 30 - RNA, 80 nt, Homo sapiens; identical to SEQ ID NO: 29

SEQ ID NO: 31 - RNA, 67 nt, Homo sapiens
gugggccucaaauguggagcacuauucugauguccaaguggaaagugcugcgacauuugagcgucac

SEQ ID NO: 32 - RNA, 67 nt, Homo sapiens; identical to SEQ ID NO: 31

SEQ ID NO: 33 - DNA, 2281 bp, Homo sapiens
agctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttaaaaaaaaaaaaaaaaaaaa

SEQ ID NO: 34 - DNA, 2168 bp, Homo sapiens
gtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttaaaaaaaaaaaaaaaaaaaa

SEQ ID NO: 35 - DNA, 3332 bp, Homo sapiens
gcgccgagccggtttccccgccggtgtccgagaggcgcccccggcccggcccggcccggcccgcgccctccgcccccgcctccccgggccggcggcggtgggcgagctcgcgggcccggccgcccccagccccagccccgccgggccccgccccccgtcgagtgcatgaggttgacgctactttgttgcacctggagggaagaacgtatgggagaggaaggaagcgagttgcccgtgtgtgcaagctgcggccagaggatctatgatggccagtacctccaggccctgaacgcggactggcacgcagactgcttcaggtgttgtgactgcagtgcctccctgtcgcaccagtactatgagaaggatgggcagctcttctgcaagaaggactactgggcccgctatggcgagtcctgccatgggtgctctgagcaaatcaccaagggactggttatggtggctggggagctgaagtaccaccccgagtgtttcatctgcctcacgtgtgggacctttatcggtgacggggacacctacacgctggtggagcactccaagctgtactgcgggcactgctactaccagactgtggtgacccccgtcatcgagcagatcctgcctgactcccctggctcccacctgccccacaccgtcaccctggtgtccatcccagcctcatctcatggcaagcgtggactttcagtctccattgaccccccgcacggcccaccgggctgtggcaccgagcactcacacaccgtccgcgtccagggagtggatccgggctgcatgagcccagatgtgaagaattccatccacgtcggagaccggatcttggaaatcaatggcacgcccatccgaaatgtgcccctggacgagattgacctgctgattcaggaaaccagccgcctgctccagctgaccctcgagcatgaccctcacgatacactgggccacgggctggggcctgagaccagccccctgagctctccggcttatactcccagcggggaggcgggcagctctgcccggcagaaacctgtcttgaggagctgcagcatcgacaggtctccgggcgctggctcactgggctccccggcctcccagcgcaaggacctgggtcgctctgagtccctccgcgtagtctgccggccacaccgcatcttccggccgtcggacctcatccacggggaggtgctgggcaagggctgcttcggccaggctatcaaggtgacacaccgtgagacaggtgaggtgatggtgatgaaggagctgatccggttcgacgaggagacccagaggacgttcctcaaggaggtgaaggtcatgcgatgcctggaacaccccaacgtgctcaagttcatcggggtgctctacaaggacaagaggctcaacttcatcactgagtacatcaagggcggcacgctccggggcatcatcaagagcatggacagccagtacccatggagccagagagtgagctttgccaaggacatcgcatcagggatggcctacctccactccatgaacatcatccaccgagacctcaactcccacaactgcctggtccgcgagaacaagaatgtggtggtggctgacttcgggctggcgcgtctcatggtggacgagaagactcagcctgagggcctgcggagcctcaagaagccagaccgcaagaagcgctacaccgtggtgggcaacccctactggatggcacctgagatgatcaacggccgcagctatgatgagaaggtggatgtgttctcctttgggatcgtcctgtgcgagatcatcgggcgggtgaacgcagaccctgactacctgccccgcaccatggactttggcctcaacgtgcgaggattcctggaccgctactgccccccaaactgccccccgagcttcttccccatcaccgtgcgctgttgcgatctggaccccgagaagaggccatcctttgtgaagctggaacactggctggagaccctccgcatgcacctggccggccacctgccactgggcccacagctggagcagctggacagaggtttctgggagacctaccggcgcggcgagagcggactgcctgcccaccctgaggtccccgactgagccagggccactcagctgcccctgtccccacctctggagaatccacccccaccagattcctccgcgggaggtggccctcagctgggacagtggggacccaggcttctcctcagagccaggccctgacttgccttctcccaccccgtggaccgcttcccctgccttctctctgccgtggcccagagccggcccagctgcacacacacaccatgctctcgccctgctgtaacctctgtcttggcagggctgtcccctcttgcttctccttgcatgagctggagggcctgtgtgagttacgcccctttccacacgccgctgccccagcaaccctgttcacgctccacctgtctggtccatagctccctggaggctgggccaggaggcagcctccgaaccatgccccatataacgcttgggtgcgtgggagggcgcacatcagggcagaggccaagttccaggtgtctgtgttcccaggaaccaaatggggagtctggggcccgttttccccccagggggtgtctaggtagcaacaggtatcgaggactctccaaacccccaaagcagagagagggctgatcccatggggcggaggtccccagtggctgagcaaacagccccttctctcgctttgggtcttttttttgtttctttcttaaagccactttagtgagaagcaggtaccaagcctcagggtgaagggggtcccttgagggagcgtggagctgcggtgccctggccggcgatggggaggagccggctccggcagtgagaggataggcacagtggaccgggcaggtgtccaccagcagctcagcccctgcagtcatctcagagccccttcccgggcctctcccccaaggctccctgcccctcctcatgcccctctgtcctctgcgttttttctgtgtaatctattttttaagaagagtttgtattattttttcatacggctgcagcagcagctgccaggggcttgggattttatttttgtggcgggcgggggtgggagggccattttgtcactttgcctcagttgagcatctaggaagtattaaaactgtgaagctttctcagtgcactttgaacctggaaaacaatcccaacaggcccgtgggaccatgacttagggaggtgggacccacccacccccatccaggaaccgtgacgtccaaggaaccaaacccagacgcagaacaataaaataaattccgtactccccaccc

SEQ ID NO: 36 - DNA, 2013 bp, Homo sapiens
agttccggcatcgcgcctggtggcggagttctgccgagtggggcgccgcggccgctattgtcccgccccctgctccgcaagattcgagcctgagcggcctgggcgtctcgagaggtgagagagttggcggcgaggtctcggcggctaagcgagcgtcggcgactgtctctccgcgagaggaggcaagttggggtccaggctccaaagccggtggccgcgtaccgcggtggagccgctgtctttgaggtctgaggagagaacagacagagtctagctctttcacccaggctggagtgaagtggtgcaatctcagctcagtgcaacctccgccccctgggttcaagtgattctcctgcctcagcctccccagtagctgggattacagtgatatcctgagagaagatgggaaagggctgcaaggttgtggtttgtggattgttatctgtggggaaaactgcaattttggagcagctcctttatggaaatcatactattggaatggaagattgcgaaacaatggaagatgtatacatggcttcagtagaaacagaccgaggagtaaaagaacagttacatctttatgacaccagaggtctacaggaaggcgtggagctgccaaagcattatttttcatttgctgatggcttcgttcttgtgtacagtgtgaataaccttgaatcctttcaaagagtggagcttctgaagaaagaaatcgataagttcaaagacaaaaaagaggtagcaattgtggtattaggaaacaaaatcgacctttctgagcagagacaagtggacgctgaagtggcacagcagtgggcaaaaagtgagaaagtaagactgtgggaggtgactgttacagatcggaaaactctgattgaaccattcactttattagccagtaaactttctcaaccccagagcaaatcaagctttcctttgcctgggaggaaaaacaaagggaactctaattctgagaactaaaaatcagtaatttccacaattgtatgttgaatagtgattgcctttaagtgtctgtgaacatggagtaatattactatttaaaataggccatttgtatctacctttggtccttaggaaaattcctaaggaagtcaattaatgcactttagatgttaaaagtatttgggctaaggttattattgcctgatatgaaataatatattcttattctcattgtttgaaacctgtctttgaaattagcacctttgttatttatgttgtacttgtgaaaacagtaaaatagtttggatagttatgcaaatgcacctatgtgtaacttccccccaaccccaagctgtttcggaagatatcataatcattctgtgtaacattatgcaaacttctaagcccaaacatgactttgtttttaaaaagttcattaatctaatgtctaggattataaaacatttttttgtgtctaaattggacccaaaacattgaacagtttggggtagtaagctaaatttcatcttgtggagattttgctaaacagactaagacccatgatttagctttgctcaaattagaatgtttagcatgagttgaggtaccaggtagtgttaagtaggttcatcacgctctaaggccgttttttccttagccagacccctgttgatagaccagatacttgagggcaaactgtttgctcctcctcttgaaaatgattaggcacttaaggacagtaaagctgtattttctggaaggaagactgtatcttctggaatagttttctagaaaactagtcatatacaataaaagtatcaaaaatattgggctctaatttgatctgacttagatgtctgagtttgtgttgtttctctaaagattttggcaagactcaagcaatgtggctgactgtaactttattaatttaaaaggtaggaagtaagctacttagtggtttcacctgtgaaataactattttgactgaaatgtaaaataagctattcaacaaagaacatattaaaacatcaaaaaaaaaaaaaaaaaaaa

SEQ ID NO: 37 - DNA, 1880 bp, Homo sapiens
tctctgtgcgttgaagccggagaccgcggcggcctcagcgaggaccctccgccccggagccgccggccggagccgcagcctctgccgcagcgcccccgccacctgtcccctccccctccgcctccgccggagccgcctcgtgcactctggggtatggccgtcaatgtgtactccacatctgtgaccagtgaaaatctgagtcgccatgatatgcttgcatgggtcaacgactccctgcacctcaactataccaagatagaacagctttgttcaggggcagcctactgccagttcatggacatgctcttccccggctgtgtgcacttgaggaaagtgaagttccaggccaaactagagcatgaatacatccacaacttcaaggtgctgcaagcagctttcaagaagatgggtgttgacaaaatcattcctgtagagaaattagtgaaaggaaaattccaagataattttgagtttattcagtggtttaagaaattctttgacgcaaactatgatggaaaggattacaaccctctgctggcgcggcagggccaggacgtagcgccacctcctaacccaggtgatcagatcttcaacaaatccaagaaactcattggcacagcagttccacagaggacgtcccccacaggcccaaaaaacatgcagacctctggccggctgagcaatgtggcccccccctgcattctccggaagaatcctccatcagcccgaaatggcggccatgagactgatgcccaaattcttgaactcaaccaacagctggtggacttgaagctgacagtggatgggctggagaaggaacgtgacttctacttcagcaaacttcgtgacatcgagctcatctgccaggagcatgaaagtgaaaacagccctgttatctcaggcatcattggcatcctctatgccacagaggaaggattcgcaccccctgaggacgatgagattgaagagcatcaacaagaagaccaggacgagtactgagggcggccgcagccctggctgactgcacggcttccccgtgcctccctccctgctccactcccacattatagtcctttcctaacacggtcggccgggtgctttgtgtcagtgctgcagcactggggagccaggcgaggggggcttgggggcatggggccggaaagcaggcagaagcccgtcctgggtggtgctggcccagttggtgggacccctgtccacacccaccctatttatttccgttgtctctctgctgtgtcgcccaacacttcccagggtgctgctgccacccgccccagccagccacctgctcctgacagccagcagctgtgtatttgacaaagtcattggtatatttttacttactggattctccttgcactttacctgttcttttccagagctgacagcacgggctccggcgcagtgtgcctggcttggcttcccttccccatggctgggggctggggtaggactcacccattctaatttattttgtcttttggcttctcagtagctaaggggaaggctgatgtcaggagagggagagggggctgaggaggtagtgctgtaggcccagggggtcagggaaagggaggggggcatgtgagggatggaaatgacctcctggcaccaggctcacccacccaaggccccctgccccagcactgaatcccagcgctgccctgaggcccccagccactccctccagcagcctggttcaccacacaaactctgcctggaccccattgtctgtctgcttcccacctgccctccccaccccctgcccctcgggcaccagcctgcatatgtgttcacttttatttaaataaacttgtgtggtaaaagtacatgccatgtgtccctcaactgaaaaaaaaaaa

SEQ ID NO: 38 - DNA, 2057 bp, Homo sapiens
ggctgcgcctgcgtggttcgtcctcacgtggccgtcaagccctctagtgccttagattccagcgagctacgcaagcaatcctggcccagccgagcttgcttccccaaatcccgtaatccttgaccttattcccccaaagaagcggcctcccgggaaggagcgccctggcggagaagactcgaacggctcccacagccgggcgttgggggaaaggcatgaagaactcttgactgacagaaacggagggtgtgtccaaagttttgaggacggccgagcggcgctccaaaacccgtcctcacagcctcgccccgttcgcctcagctacaacaaatcatcgtcaacctgttccaccttctccagtctggtagcaaaaaggggtgtctcagaatctccggcctgtgaaactgtgaggggattcggccaagacgtcctcttccctctgcctcccacccaggccactcttcacctccaccatgagcctggacatccagagcctggacatccagtgtgaggagctgagcgacgctagatgggccgagctcctccctctgctccagcagtgccaagtggtcaggctggacgactgtggcctcacggaagcacggtgcaaggacatcagctctgcacttcgagtcaaccctgcactggcagagctcaacctgcgcagcaacgagctgggcgatgtcggcgtgcattgcgtgctccagggcctgcagaccccctcctgcaagatccagaagctgagcctccagaactgctgcctgacgggggccggctgcggggtcctgtccagcacactacgcaccctgcccaccctgcaggagctgcacctcagcgacaacctcttgggggatgcgggcctgcagctgctctgcgaaggactcctggacccccagtgccgcctggaaaagctgcagctggagtattgcagcctctcggctgccagctgcgagcccctggcctccgtgctcagggccaagccggacttcaaggagctcacggttagcaacaacgacatcaatgaggctggcgtccgtgtgctgtgccagggcctgaaggactccccctgccagctggaggcgctcaagctggagagctgcggtgtgacatcagacaactgccgggacctgtgcggcattgtggcctccaaggcctcgctgcgggagctggccctgggcagcaacaagctgggtgatgtgggcatggcggagctgtgcccagggctgctccaccccagctccaggctcaggaccctgtggatctgggagtgtggcatcactgccaagggctgcggggatctgtgccgtgtcctcagggccaaggagagcctgaaggagctcagcctggccggcaacgagctgggggatgagggtgcccgactgctgtgtgagaccctgctggaacctggctgccagctggagtcgctgtgggtgaagtcctgcagcttcacagccgcctgctgctcccacttcagctcagtgctggcccagaacaggtttctcctggagctacagataagcaacaacaggctggaggatgcgggcgtgcgggagctgtgccagggcctgggccagcctggctctgtgctgcgggtgctctggttggccgactgcgatgtgagtgacagcagctgcagcagcctcgccgcaaccctgttggccaaccacagcctgcgtgagctggacctcagcaacaactgcctgggggacgccggcatcctgcagctggtggagagcgtccggcagccgggctgcctcctggagcagctggtcctgtacgacatttactggtctgaggagatggaggaccggctgcaggccctggagaaggacaagccatccctgagggtcatctcctgaggctcttcctgctgctgctctccctggacgaccggcctcgaggcaaccctggggcccaccagcccctgccatgctctcaccctgcatatcctaggtttgaagagaaacgctcagatccgcttatttctgccagtatattttggacactttataatcattaaagcactttcttggcaggaaaaaaaaaaaaaaaaaaaa

SEQ ID NO: 39 - DNA, 5916 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 40 - DNA, 1691 bp, Homo sapiens
tgcggccgcgtttccgtggagacagccgagcctgcggaaggcggcggcggcggcacctgcgatcagcggctggggcaggttatggtagtgcggactgcggtgtgagcagagcggccacggggcccgccatgcgccggcggccctgacatgggcgccagcgggtccaaagctcggggcctgtggcccttcgcctcggcggccggaggcggcggctcagaggcagcaggagctgagcaagctttggtgcggcctcggggccgagctgtgccccccttcgtattcacgcgccgcggctctatgttctatgatgaggatggggatctggctcacgagttctatgaggagacaatcgtcaccaagaacgggcagaagcgggccaagctgaggcgagtgcataagaatctgattcctcagggcatcgtgaagctggatcacccccgcatccacgtggatttccctgtgatcctctatgaggtgtgaccctgggaggtggcagacagaagcaccccctgccccggcaagaaactcccaggctcaatcaaggtgtggcttccattgaggagcccaggctggggccacaaccctgaataaactctgttggcccataaccttcagctgtgagcgggtcggtcccacagtattggttgggtgttggtttgtgtgtggacaagaggtggttggtgggtggtgaaggctaatggcagagttagcaccccactctcccaagccacccctgcaagcagcatagcagggcatataccagtcaggaatgcccgttacctggttccttgcctggtctgctttcttccaagtttgcctggggcctagccctgctagaggctacagcactttacaagcaaggtatgctttcttccagcccctaggctgtgggcactgtatacaagtaggaacttcctttccttcacttcccttttaacccctagtcagagcatttcagccgtttgctacctcgattcctcctgtgttggacagaggctgggggcagtgccagcctgattcttccgacctacctgccatttgttcccgccttcagatggatggacagtttgctggctattgataggagtggggactgggtgggggcttctccctctacccagggctgggctgatccccctactgcaactaactgttgccccccaaccccgaacccccagttgaggagttgagagagtgcaggctggggtcaggacaggctgcggatgcttgtgcctatggggagttactccaacccacctattctgtctaatctccatggctttgcaccaaatcctccacccctccaattgggaggggactgttcaccaccttgtggtaagggacaacaccctaaggctggtgccagtagttatgagtagcctaccaccccctcccttacagtaacccccaccccttcaggatcagtcaagggaaagcactagaacccctgggtagggaaagaaaggagggaaaaaccataaaaggaatacttataatgtgaaggtttgtaaatagtccatgatgatgtcgtggcagagtctgatttctatatagaggtgactttttttttaagtactgtgcaagctctgtgcttctataatgtgggaaatggcttggggaggatggcccctagcttaggaagactgttgtgttatttgttcaatttcaataaaatgatttgtagatcctgc

SEQ ID NO: 41 - DNA, 1464 bp, Homo sapiens
ccctcttccgggccgcgagccccctgcgcgccgctttggggctgcgctcactcgtgtgcgcgctcgtccgcccgccagtcctctcaacgcgcgcttggccgcccgacgacgcgggagccgcacgcgccggacgaggctcgctgcgctccctgttgcccagcgcgggcccgttgaggcggagccctcagttcccggccaggacacggtctgggccgccgaatctccggccgaagagcggcggcggcagcggcgggaaaaaaatgaagaatgaaattgctgccgttgtcttctttttcacaaggctagttcgaaaacatgataagttgaaaaaagaggcagttgagaggtttgctgagaaattgaccctaatacttcaagaaaaatataaaaatcactggtatccagaaaaaccatcgaaaggacaggcctacagatgtattcgtgtcaataaatttcagagagttgatcctgatgtcctgaaagcctgtgaaaacagctgcatcttgtatagtgacctgggcttgccaaaggagctcactctctgggtggacccatgtgaggtgtgctgtcggtatggagagaaaaacaatgcattcattgttgccagctttgaaaataaagatgagaacaaggatgagatctccaggaaagttaccagggcccttgataaggttacctctgattatcattcaggatcctcttcttcagatgaagaaacaagtaaggaaatggaagtgaaacccagttcggtgactgcagccgcaagtcctgtgtaccagatttcagaacttatatttccacctcttccaatgtggcaccctttgcccagaaaaaagccaggaatgtatcgagggaatggccatcagaatcactatcctcctcctgttccatttggttatccaaatcagggaagaaaaaataaaccatatcgcccaattccagtgacatgggtacctcctcctggaatgcattgtgaccggaatcactggattaatcctcacatgttagcacctcactaacttcgtttttgattgtgttggtgtcatgttgagaaaaaggtagaataaaccttactacacattaaaagttaaaagttcttactaatagtagtgaagttagatgggccaaaccatcaaacttatttttatagaagttattgagaataatctttcttaaaaaatatatgcactttagatattgatatagtttgagaaattttattaaagttagtcaagtgcctaagtttttaatattggacttgagtatttatatattgtgcatcaactctgttggatacgagaacactgtagaagtggacgatttgttctagcacctttgagaatttactttatggagcgtatgtaagttatttatatacaaggaaatctattttatgtcgttgtttaagagaattgtgtgaaatcatgtagttgcaaataaaaaatagtttgaggcatgacaaaa

SEQ ID NO: 42 - DNA, 6067 bp, Homo sapiens
aaagggggggaacctagagtcggtgggggggaagcgatgtttgcccgtcagtcgagtccggagtgaggagctcggtcgccgaagcggagggagactcttgagcttcatcttgccgccgccacggccaccgcctggacctttgcccggagggagctgcagagggtccatcgccgccgtcctctggagggcagcgcgattgggggcccggacctccagtccgggggggatttttcgtcgtccccctccccccaaccagggagcccgagcggccgccaaacaaaggtaccagtcgccgccgcgggaggaggaggagccggagcctctgcctcagcagccgctggacccgccgcccttcttccccatctctcccccgggcctgctggttttgggggggagaaggagagaggggactctggacgtgccagggtcagatctcgcctccgaggaaggtgcagctgaacctggtgttttagaggataccttggtcccagagtcatcatgaaggcccttgatgagcctccctatttgacagtgggcactgatgtgagtgctaaatacagaggagccttttgtgaagccaagatcaagacagcaaaaagacttgtcaaagtcaaggtgacatttagacatgattcttcaacagtggaagttcaggatgaccacataaagggcccactaaaggtaggagctattgtggaagtgaagaatcttgatggtgcatatcaggaagctgttatcaataaactaacagatgcgagttggtacactgtagtttttgatgacggagatgagaagacactgagacgatcttcactgtgcctgaaaggagagaggcattttgctgaaagtgaaacattagaccagctcccactcaccaaccctgagcattttggcactccagtcataggaaagaaaacaaatagaggaagaagatctaatcatataccagaggaagagtcttcatcatcctccagtgatgaagatgaggatgataggaaacagattgatgagctactaggcaaagttgtatgtgtagattacattagtttggataaaaagaaagcactgtggtttcctgcattggtggtttgtcctgattgtagtgatgagattgctgtaaaaaaggacaatattcttgttcgatctttcaaagatggaaaatttacttcagttccaagaaaagatgtccatgaaattactagtgacactgcaccaaagcctgatgctgttttaaagcaagcctttgaacaggcacttgaatttcacaaaagtagaactattcctgctaactggaagactgaattgaaagaagatagctctagcagtgaagcagaggaagaagaggaggaggaagatgatgaaaaagaaaaggaggataatagcagtgaagaagaagaagaaatagaaccatttccagaagaaagggagaactttcttcagcaattgtacaaatttatggaagatagaggtacacctattaacaaacgacctgtacttggatatcgaaatttgaatctctttaagttattcagacttgtacacaaacttggaggatttgataatattgaaagtggagctgtttggaaacaagtctaccaagatcttggaatccctgtcttaaattcagctgcaggatacaatgttaaatgtgcttataaaaaatacttatatggttttgaggagtactgtagatcagccaacattgaatttcagatggcattgccagagaaagttgttaacaagcaatgtaaggagtgtgaaaatgtaaaagaaataaaagttaaggaggaaaatgaaacagagatcaaagaaataaagatggaggaggagaggaatataataccaagagaagaaaagcctattgaggatgaaattgaaagaaaagaaaatattaagccctctctgggaagtaaaaagaatttattagaatctatacctacacattctgatcaggaaaaagaagttaacattaaaaaaccagaagacaatgaaaatctggatgacaaagatgatgacacaactagggtagatgaatccctcaacataaaggtagaagctgaggaagaaaaagcaaaatctggagatgaaacgaataaagaagaagatgaagatgatgaagaagcagaagaggaggaggaggaggaagaagaagaagaggatgaagatgatgatgacaacaatgaggaagaggagtttgagtgctatccaccaggcatgaaagtccaagtgcggtatggacgagggaaaaatcaaaaaatgtatgaagctagtattaaagattctgatgtcgaaggtggagaggtcctttacttggtgcattactgcggatggaatgtgagatacgatgaatggattaaagcagataaaatagtaagacctgctgataaaaatgtgccaaagataaaacatcggaagaaaataaagaataaattagacaaagaaaaagacaaagatgaaaaatactctccaaaaaactgtaaacttcggcgcttgtccaaaccaccatttcagacaaatccatctcctgaaatggtatccaaactggatctcactgatgccaaaaactctgatactgctcatattaagtccatagaaattacttcgatccttaatggacttcaagcttctgaaagttctgctgaagacagtgagcaggaagatgagagaggtgctcaagacatggataataatggcaaagaggaatctaagattgatcatttgaccaacaacagaaatgatcttatttcaaaggaggaacagaacagttcatctttgctagaagaaaacaaagttcatgcagatttggtaatatccaaaccagtgtcaaaatctccagaaagattaaggaaagatatagaagtattatccgaagatactgattatgaagaagatgaagtcacaaaaaagagaaaggatgtcaagaaggacacaacagataaatcttcaaaaccacaaataaaacgtggtaaaagaaggtattgcaatacagaagagtgtctaaaaactggatcacctggcaaaaaggaagagaaggccaagaacaaagaatcactttgcatggaaaacagtagcaacagctcttcagatgaagatgaagaagaaacaaaagcaaagatgacaccaactaagaaatacaatggtttggaggaaaaaagaaaatctctacggacaactggtttctattcaggattttcagaagtggcagaaaaaaggattaaacttttaaataactctgatgaaagacttcaaaacagcagggccaaagatcgaaaagatgtctggtcaagtattcagggacagtggcctaaaaaaacgctgaaagagcttttttcagactctgatactgaggctgcagcttccccaccgcatcctgccccagaggagggggtggcagaggagtcactgcagactgtggctgaagaggagagttgttcacccagtgtagaactagaaaaaccacctccagtcaatgtcgatagtaaacccattgaagaaaaaacagtagaggtcaatgacagaaaagcagaatttccaagtagtggcagtaattcagtgctaaatacccctcctactacacctgaatcgccttcatcagtcactgtaacagaaggcagccggcagcagtcttctgtaacagtatcagaaccactggctccaaaccaagaagaggttcgaagtatcaagagtgaaactgatagcacaattgaggtggatagtgttgctggggagctccaagacctccagtctgaagggaatagctcgccagcaggttttgatgccagtgtgagctcaagcagtagtaatcagccagaaccagaacatcctgaaaaagcctgtacaggtcagaaaagagtgaaagatgctcagggaggaggaagttcatcaaaaaagcagaaaagaagccataaagcaacagtggtaaacaacaaaaagaagggaaaaggcacaaatagtagtgatagtgaagaactttcagctggtgaaagtataactaagagtcagccagtcaaatcagtttccactggaatgaagtctcatagtaccaaatctcccgcaaggacgcagtctccaggaaaatgtggaaagaatggtgataaggatcctgatctcaaggaacccagtaatcgattacccaaagtttacaaatggagttttcagatgtcggacctggaaaatatgacaagtgccgaacgcatcacaattcttcaagaaaaacttcaagaaatcagaaaacattatctgtcattaaaatctgaagtagcttccattgatcggaggagaaagcgtttaaagaagaaagagagagaaagtgctgctacatcctcatcctcctcttcaccttcatccagttccataacagctgctgttatgttaactttagctgaaccgtcaatgtccagcgcatcacaaaatggaatgtcagttgagtgcaggtgacagcaggacttgctaaagcactttgcacttaatggctgttgagggccactttttttttatactgcacagtggcacaaaaaaatatcagacaagcactattttatatttaaaaattgtttcttgacaagctgacttggcacttaagtgcacttttttatgaagaaaaagtacaatgaactgcttttcctcaagcaataattgtttccaacttgtctgggaattgtgtgtctggtaactggaaggccttccactgtggcaaatggaggcttttcactgcctgtagagacaatacagtaagcatagttaaggggtgggtcagaacatgttaagataacttactgtatatgtattcccttgtattttgttaaagctggaacatttgatatttttccatttatttatgaaaaaatatgaacctattttcatttgtacaaggtaattgttttttaaagcaagtcaccttagggtggctttaattgtataagtcaagcacatgtaataaattcaaaacctgcagttaacaggatattagacatcaatcctggtaaccaaatattaaagattctctttaaaaaagactgaacatgtttacaggtttgaattaggctaaaaggtcttgcagtggcttttcatggcccttcaaattggaatggaactactgtactttgccatttttctataaatcagtatttttttttaattttgatatacattgtgtgaaaaaagaaaatggctaataaactgtattaaatcttaaacaatgtataaagattgtacttagccagttcaaagtgtatatttattcataatgaattataacagttatatttttgtgttttcttgtaaatgtttcttttcccttaaatacagataattcatttgtattgcttattttattatgagctacaacaaaaggacttcaggaacaagtaatgtattagtatggttcaagattgttgataggaactgtctcaaaaggatggtggttattttaaatataaatagctaatgggggtggtaggcctataaaattaaatgccttgtataaaatccaaaatgaatgcaaaattgttttcacttgtattgactttatgttgtatgattccaatctctgttctgtttggcacttgtatttaattcttcacctttgtaagacatttgtatattgtggatgtgttcattcaagctatttaatatctggcactgttaatacacagtactttattgtacagactgttttactgttttaattgtagttctgtgtactttttttggatggggctggcatgttttctttgtttcctggcaatacgacgtgggaatttcaatgcgttttgttgtagatgctaacgtgtcagaatcctttacattcaacttttctaagaaaagcattttcagtcttgtagtgtgtgcttacagtaactaattttgttgaaaatggtttcaagttattcaaatttgtacaggactgtaaagatttgttgacagcaaaatgttgaagaaaaaagcttatagaataaaagctataaagtatatattaggatctgcaaacaatgaagaattatgtaatatattgtacaaatgtaagcaaaggctctgaaataaaatgccatagtttgtgaatccttga

SEQ ID NO: 43 - DNA, 2500 bp, Homo sapiens
cccaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgttttgtactcacggcggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcataaaaaaaaaaaaaaaaaaaaaaaaa

SEQ ID NO: 44 - DNA, 7536 bp, Homo sapiens
ccttttcacgcgcgtcgcgagctaacggactcggcggcggcggcggcggcggcctgcgccccacccgcaccccatctggaccgcatcgctgaatgtgcccggacctgcgccttctgggtctctgaaagaagatgaatttggctgagatttgtgataatgcaaagaaaggaagagaatatgcccttcttggaaattacgactcatcaatggtatattaccagggggtgatgcagcagattcagagacattgccagtcagtcagagatccagctatcaaaggcaaatggcaacaggttcggcaggaattattggaggaatatgaacaagttaaaagtattgtcagcactttagaaagttttaaaattgacaagcctccagatttccctgtgtcctgtcaagatgaaccatttagagatcctgctgtttggccaccccctgttcctgcagaacacagagctccacctcagatcaggcgtcccaatcgagaagtaagacctctgaggaaagaaatggcaggagtaggagcccggggacctgtaggccgagcacatcctatatcaaagagtgaaaagccttctacaagtagggacaaggactatagagcaagagggagagatgacaagggaaggaagaatatgcaagatggtgcaagtgatggtgaaatgccaaaatttgatggtgctggttatgataaggatctggtggaagcccttgaaagagacattgtatccaggaatcctagcattcattgggatgacatagcagatctggaagaagctaagaagttgctaagggaagctgttgttcttccaatgtggatgcctgactttttcaaagggattagaaggccatggaagggtgtactgatggttggacccccaggcactggtaaaactatgctagctaaagctgttgccactgaatgtggtacaacattcttcaacgtttcgtcttctacactgacatctaaatacagaggtgaatctgagaagttagttcgtctgttgtttgagatggctagattttatgcccctaccacgatcttcattgatgagatagattctatctgcagtcgaagaggaacctctgatgaacatgaggcaagtcgcagggtcaagtctgaactgctcattcagatggatggagttggaggagctttagaaaatgatgatccttccaaaatggttatggtattggctgctactaatttcccgtgggacattgatgaagctttgcgaagaaggttagaaaaaaggatatatatacctctcccaacagcaaaaggaagagctgagcttctgaagatcaaccttcgtgaggtcgaattagatcctgatattcaactggaagatatagccgagaagattgagggctattctggtgctgacatcactaatgtttgcagggatgcctctttaatggcaatgagacggcgtatcaatggcttaagtccagaagaaatccgtgcactttctaaagaggaacttcagatgcctgttaccaaaggagactttgaattggccctaaagaaaattgctaagtctgtctctgctgcagacttggagaagtatgaaaaatggatggttgaatttggatctgcttgaatttctgtcagctctttaatttctggtatttttgttgataaaatacgaagaaattcctgcaatttttaaaaaacaagtttggaatttttttcagtggagtggttttcgcttaaaggaaaaaaaaatctaaaactgcgaagaatactaaatgtagttgagaaataattgatggcgagagtttgctagtctccctccccggctttgtgctggtattccacgtattcctgcattaatattgcacacccaaaccagtctatcagggaggctgaagcaagggcgcagtgtgatattttaggaatacagaagatttagaaatacccctatttctcatttgcagtttttttttccaattctgtgctctgtcaacatgagggacctatctatgtatgttgacttttaacatcaaaattggatttgtgtcaaacattcattgttaagagaagaatgacagtatattttggaggaaataatgaatttactaattaaacctttagaatttatgacttactgttagagtctgtcatatggttagaatttttacttccgctacccctgccatttcttctgctagctacttcataatatcttgagctttactgaggaatattctcacgctctgtggtatttgaatcattttgccaggtcatttctctgtctttagtattttttgctggtgcttcttacatttaatatggaaaggtgggaagaatattactgcattagatgtaattcttcattctagacttccaagtttgttttcacttttttgtgtgtgcgtgaaggagtctgtgtcacccaggctgtgtagtgcagtggttgatcttggctcactgcaacctctgcctcctagattcaagcaattctcctgtctcagcctcccaagtagctgggattacaggtgcgcaccaccatgcctggctgtgttttcacttttctttcaacatgttcaaccagatatatagccattatttttctcagctccagcattgtttgatttttcttgagtttgattttagtatttgagataaatacttttacattctaaacaagtccactctctgtggctaacgcaaaacaaatgaaatctttattgttttccaaacagctagtttaacaaaacagcatcatacatagtgaatgatgttcattggaaaattctaaaatttgtccttgtctaggttgagaacttttacacacactaagataaagatagaaatctgacatgctcactcaattcagcaggaattacacattagaaagaagccagaaaaataaatggcatatatccaatcacaagtaaatgatcctggcgttagtttttatgattacatgtgtctcattaggcaatttatgctttaatggtcaagcttttaaaaatttgtatttgataacatcctgaattctcagtttcgaatagtgcctactggtttaaaactaaaaataatacagctttttggacatttaaccaagatactaagaaggttttttttaaaaaaagagatttgattatttttccctgctaaaaactgtaaatgccttatgttcttttcagataacttaagtctgacctaaactccagtattcatctgatgctgtaaattgcccttctttctgagacacagattataagatgccagatcataagacatcatgattttattgtaattgaattcttcctaaaaattgagaggtttccttttattaacttttaaaataaagaaataagtagtttcattacgattattttgcaaactattgccagtcagaaatgcactttttttttccctgaagttttaggagccgtcactaaaacattagtcttgtgattgttaaaacttgtttgtaatgggttggtgcaaaagtaattgtggtttttccattactttcaatggcaaaaaccgcaattacttttgcaccagcctaacaatagttgattagttagaccttttctgggttttgtattgattatcttggtgtgcatttaattatttttctgaattcttcatggataatgacatagtaattgtgattcttttaataccagttaagcagtatttggcaacttaaacttcctgggagcctaactttactatgttaagtgagtcaggtgtgctttttatttcccttgtttctcattttgccctgtcagtggatggtagatgctttgtatatcttaaatcccttaaaggatcttaaagacatccctcaggtgttctatttaacttttattttattttattttatttatttatttattttgagactgagtcttgctctgtcgcccaggctggagtgcagtggcatgatctcggctcactgcaacttctgcctcccaggttcaagccattctcctgcctcggcctcctgagtagctgggattacagttgccgccacacccggcttatttttttgtatttttagtagaggcagagtttcaccatgttggccaggctagtctcgaactcctgacctcagatgatccgcccacattggcctcccaaagtgctgggattacaggtgtgatccaccgcacctggccctaacttttaatatacaacacacacacacacacacacacacacacacacacacacacacacacacacacacacactatttcagaagacagtgtgttgccttacccagaatgagtgctaggattacaggcgtgagacagacacacatacacacacatacacacacacagagtctttattgcagaagacagtgtgttgccttataggcgtgagacacacacacacacacacacacacacacacacacacacagtctttattgcagaagacagtgtgttgccttaccagaatgagtgcttggattacaggcgtgagccactgtgcccagccctaacttttaatgtacatcacacacacactcacactcacatacacacacacacacacactctgactgtctttattgcagaagacagtgtgttgccttacccagaatgagattgaattgttttgcttcgttttgttttgttattcagtgttgcggtagcagatgcattatcaaaggaaaaatatttggctcctttaattcctctgaaaacatgagtattttgagttctgcagcacaatgactgtaggactaagctaagtctgctttgcagatatctgatcagatagtcccttcattctgtagacgtgtattggttggtccaagacacagtgagtaggagctctgtggaccaagacaaagctggactagagagtacagttcaaacttggcagtttctctaacgactctgtatagcttctggcttctactactgaaacaagagtttagatcactgatggagaggcatagtaatctgtttgtgctttggaaaaatatataaaagtttttttcccctattttttgcactttaaatctgttttgaaattagaactgatatacatttatttgaataatgtgtaactattatggatctattttaatgaacaatttttaccatttcccaagctgcctgtttattataagcatgacatgtttactataaaccttttgcccccataatttctttttttaaaggaaattaatattagtaaaataaacacctctttaatggaagctgcaaccttctagtgatccaagtagacaatagatggtggcatcacagactttatctacacactttcgggtctgaccactacctcccacaatacctagccattttggaaggggaaaacatgcggtggtctagctgtatagctcagggcttaatttcagcttctgagattgtgatgtcatatttcactctcaaaacataggctgaaagcacgaattactcaaaaagtaagcaaaccaatacctggtgaatctatggacagtcatacacatacatcaggggaaaatgtgtgtgtacaacccaaatttacagtatgattgtcattctttgactttgttttgtatagcctgactctgttgaacatgaaattattagtactctaggttttggacagcttgagttcatttgaattccttccttaggaataagtttttatatacactgctaaatgtgtgatgagaatcataaaacactaaccagctgaggtagctgtgattcactttccccccaccctaacttgagataaaatgaaggactaggcaagtatttcatgttgtgtgagtggacttcggttccttcagtattgtctaggttattgagtctttctttgcctaatagtggattcccactcttaagataacttttattagtgataaatcagtttagggtatattctgtatgacaggcataaaatgttaagggtgaatgctggccttttccaagaaaaggccaccttaacttgtatgaggaaaaaatcctaactattctcttttttgtatctttttttccgtaactgttttgattgtatattttaaagaaaccacttaatttgtgatgcacgtaatatttgtgtgaacctgagaatatgtcacaataggaaaaagcagaaattatacttaggggacatgttaggggggtaaaaatatttaagcctcgaatgttttactgtcatctccactaactatttttacagaaaaagctaaaaactctgttgtaattattgtaagtttacttatttatacttttaaattaggcttttcatacttaaatttttttgacatttgcttttaatatttgtttcttaatgtggaaattgtgtattttaataatcaaattattaggataatagatatatttttaaacattcacctcattaacaaatagatctttgaatttttattaggttttttggctccagacaactgtttagctttaatgatatttctaaattcccagtgacttattaataaaaacaggaaaaatatttaggtaatgtcataaaatttattttacctttctcattttctgagaaaataaatgaaaaaaaccctagatattgctttattaccaacagtgtgtaggtttttgtacatatggaaatttgacacaaaaaaatagggaatttgtatagagaagtttccctcttataaaaggactcccatttgattgttcgaaactataaaatgcacttttactttaccatatctgaaatgacaaaatatcgccctttggaaaacctgactctttgcacgtgtaattcccagagtctacctcagttaaccaggcttagttttaggcaggaatgaattgaattaaattcagttcatcatctatgcagatttgtttcttttaagcacatccttccctcctgctgttgccctcctcccattaacttttctttttaatcttgaaattgtttaaaatattccatctttctttctctagcaaagtgtttgtattccaaataaggcctctgtgaaatgtctgaattacttttcccgtctttgttatggtcagcttcattatttggatgtattgcattcaaagcagcagttccaaacataacacacatctattttcttagagttttgtaaatacaaactaacctgatgacattaaaaattgtggatcctacatgttcctatgttcattctctaaaaacctgagtaactttatgaaaacacacaaacctggaaaaacatcacatttttgtcacatttttactgacaaatgtatattcatatgatggtacggcagcagggagtggcccccagttaacatggctgtgagtggacacagtgtctcgcaggatcactgcatgttatgatggcttgtaagtgcgttgttaagacttttgtttcagtgtttgtctcccagtatttgaacctaatttaaagaaaaagacgtttccaagttgtatttattaaatgtgtttttccttaccttttgtgctgctactttgctaatctcattagcttagctgtgtttgtgcataggttatatttggtaataaatttatagagtgttggttgtcaaaaaaaaaaaaaaaaaaaaaaa

SEQ ID NO: 45 - DNA, 4304 bp, Homo sapiens
cacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccaaaaaaaaaaaaaaaa

SEQ ID NO: 46 - DNA, 1724 bp, Homo sapiens
aaaaaaattttcttccgccgtccgccggtggcgaggcccaggctgtcgccgggtgtgcagcggcgtcgcggccagtagagggattctgggtaacggcccggacccccggctgggcttctggctcggcgcagcaggttccattcacgccaagtctgttggcagtggcagttgtagggccaagggcggttgtaggacccggagcagccggacatggaacaaccgtggccgcctccgggaccctggagcctccctcgggccgagggtgaggctgaggaagagagtgacttcgacgtgttccccagttctccccgctgcccgcagctgccaggcggcggcgcccagatgtatagccatggaattgaattggcttgccaaaagcagaaagagtttgtgaagagctctgtggcgtgcaaatggaatcttgctgaagctcaacagaaacttggtagcttagcactgcataattctgagtccttggatcaggagcatgccaaagcacaaacagcagtatcagaactgaggcaacgggaagaagagtggcgacagaaagaagaagctctagtacaaagagagaagatgtgtctgtggagcacggatgccattagcaaggatgtttttaataagagttttattaatcaagataaaagaaaagacacagaagatgaagataaatcagaatcatttatgcaaaaatatgagcaaaaaatcagacattttggtatgttgagtcgatgggatgatagccagagatttttgtctgaccatccataccttgtatgtgaagaaactgctaaatatcttattttatggtgttttcacctggaagctgagaagaaaggggctttaatggaacaaatagcacatcaagctgttgtaatgcagtttattatggaaatggccaaaaactgtaatgtggatccaagagggtgttttcgtttatttttccagaaagccaaagcagaggaagaaggttattttgaagcattcaaaaatgaacttgaagctttcaagtcaagagtaagactttattctcaatcacaaagttttcaacctatgacagttcagaatcatgttccccattctggtgttggatctataggtttattagaatccttaccacagaatccagattatcttcagtattctatcagtacagctctctgcagcttaaactcggtggtacataaagaagatgatgaacccaaaatgatggacactgtataatttggttaagactgctgaggccaagtgctattttgttacaagaaaggaagaacttggctattttcttgacacttttatgggtgctgcactttatttttgttcggtttttgatgggagggaaagagtactgaaatgttttgtaaattttttttaatgtgctgctaggttttttgttttgttttgttctgaagagaagagtggtaccatatgttgcaggaagtcaaactggactttttgtggctactaaatttgcttttaatcttattgttctcaattttggaatcaagtatgaaaatctgcacaaatgcaatgtttacaagaactggttgattctgggaggcatctgctacagtctctttttatatggatatgtacatgtcctattctacaaaaatgattaaagataaaaacatacttgtatcccactgctactttagctgtcaaatttggtgtttcatcacattaaaagcaataaatcagtaaaaaaaaaaaaaaaaaaa

SEQ ID NO: 47 - DNA, 4706 bp, Homo sapiens
cgcggccccggaggcagcagcagcggcggcggcagccggagcagtaggcacccgagcagcgccagcggccgagcgggcggcttcctggcctgggcgctccggtggcggcggaggtgcgcgcggagccatggttatcatgtcggagttcagcgcggaccccgcgggccagggtcagggccagcagaagcccctccgggtgggtttttacgacatcgagcggaccctgggcaaaggcaacttcgcggtggtgaagctggcgcggcatcgagtcaccaaaacgcaggttgcaataaaaataattgataaaacacgattagattcaagcaatttggagaaaatctatcgtgaggttcagctgatgaagcttctgaaccatccacacatcataaagctttaccaggttatggaaacaaaggacatgctttacatcgtcactgaatttgctaaaaatggagaaatgtttgattatttgacttccaacgggcacctgagtgagaacgaggcgcggaagaagttctggcaaatcctgtcggccgtggagtactgtcacgaccatcacatcgtccaccgggacctcaagaccgagaacctcctgctggatggcaacatggacatcaagctggcagattttggatttgggaatttctacaagtcaggagagcctctgtccacgtggtgtgggagccccccgtatgccgccccggaagtctttgaggggaaggagtatgaaggcccccagctggacatctggagcctgggcgtggtgctgtacgtcctggtctgcggttctctccccttcgatgggcctaacctgccgacgctgagacagcgggtgctggagggccgcttccgcatccccttcttcatgtctcaagactgtgagagcctgatccgccgcatgctggtggtggaccccgccaggcgcatcaccatcgcccagatccggcagcaccggtggatgcgggctgagccctgcttgccgggacccgcctgccccgccttctccgcacacagctacacctccaacctgggcgactacgatgagcaggcgctgggtatcatgcagaccctgggcgtggaccggcagaggacggtggagtcactgcaaaacagcagctataaccactttgctgccatttattacctcctccttgagcggctcaaggagtatcggaatgcccagtgcgcccgccccgggcctgccaggcagccgcggcctcggagctcggacctcagtggtttggaggtgcctcaggaaggtctttccaccgaccctttccgacctgccttgctgtgcccgcagccgcagaccttggtgcagtccgtcctccaggccgagatggactgtgagctccagagctcgctgcagtggcccttgttcttcccggtggatgccagctgcagcggagtgttccggccccggcccgtgtccccaagcagcctgctggacacagccatcagtgaggaggccaggcaggggccgggcctagaggaggagcaggacacgcaggagtccctgcccagcagcacgggccggaggcacaccctggccgaggtctccacccgcctctccccactcaccgcgccatgtatagtcgtctccccctccaccacggcaagtcctgcagagggaaccagctctgacagttgtctgaccttctctgcgagcaaaagccccgcggggctcagtggcaccccggccactcaggggctgctgggcgcctgctccccggtcaggctggcctcgcccttcctggggtcgcagtccgccaccccagtgctgcaggctcaggggggcttgggaggagctgttctgctccctgtcagcttccaggagggacggcgggcgtcggacacctcactgactcaagggctgaaggcctttcggcagcagctgaggaagaccacgcggaccaaagggtttctgggactgaacaaaatcaaggggctggctcgccaggtgtgccaggcccccgccagccgggccagcaggggcggcctgagccccttccacgcccctgcacagagcccaggcctgcacggcggcgcagccggcagccgggagggctggagcctgctggaggaggtgctagagcagcagaggctgctccagttacagcaccacccggccgctgcacccggctgctcccaggccccccagccggcccctgccccgtttgtgatcgccccctgtgatggccctggggctgccccgctccccagcaccctcctcacgtcggggctcccgctgctgccgcccccactcctgcagaccggcgcgtccccggtggcctcagcggcgcagctcctggacacacacctgcacattggcaccggccccaccgccctccccgctgtgcccccaccacgcctggccaggctggccccaggttgtgagcccctggggctgctgcagggggactgtgagatggaggacctgatgccctgctccctaggcacgtttgtcctggtgcagtgagggcagccctgcatcctggcacggacactgactcttacagcaataacttcagaggaggtgaagacatctggcctcaaagccaagaactttctagaagcgaaataagcaatacgttaggtgttttggctttttagtttatttttgttttatttttttcttgcactgagtgacctcaactttgagtagggactggaaactttaggaagaaagataattgaggggcgtgtctgggggcgggggcaggaggggagcggggtggagggaacacgtgcagtgccgtggtgtggggatctcggcccctctctctgggttcgtcgtggttgagatgattacctcggacgtctacggaaacgagcgggcgcattgttgtccgcttgtgtgtgtgtgtgtgtgtgtgtgtgtgcgcgtgcattgattactatccatttctttagtcaacgctctccacttcctgatttctgctttaaggaaaactgtgaactttctgcttcatgtatcagttttaaagcagcccaggcaaagatcatctacagattctaggaattctctcccctgaaatcaaaacctggaagacttttttttcttattttagttgagaagtttcataaactgctcaaggattagttttccaggactctgcggaggaacggcaggaagaacctcagagagggcagaggtgacttcaaagtgctggggactccgtcctgagggtcacttggccctgagcccctgcgtgcccttgcggaagcccagaagcttcttcctgctgcacctcccgtttccgctgctgctgacgtttatgcatttcatgatggggtccaacaagaacacctgacttgggtgaagttgtgcaatattggaggctgactgtagggctgggcagctgggagacaggctcatggctcatggctcatggctcagggcggtgcctgccctgggccgggacccccctccccaccccccacctaggctttttgggttttgttcaaggaaggtaaagtgagaggtttaggtcagtgtttttaagtttttgttttttttttaaagcaaatcctgtatatgtatctacatgggagacaggtagacactacttatttgttacattttgtactatacgtttgtgttccaggtttcagcttccctcgctcctgttgttaagaagcgtccctgtcagcacaggtgtgcattgaggaaggggccccagggccttcgctccctcagcactggggtggaggcggcaggaaggggcggcccttacctggcaggtctgggcgcacctttagcaggtggactccgtggggctccaccagccagaagcctctggaaggcaacgaaggcaatgctgctccctgagtccagtccccgcccccaaacccagcccaggtgccttcagctacttcggcttcttaaaccctgcagtgttaaacagaggcattgagaaaggggaaaggcgggtatttttaaaagccaaagattgacccagttacttgagggtagggaggcgggcccagtgcaggaggctgcatccctggcctgctggtgcccaccgggggctgtgcctgtgccgggccgcagggaagctggctgcccccattcctgctgctgctgctgctgctgctctgtggctgtttcaaagactgggcgaaaggctgtccggagggcagaccaggtgccttgccgcagagaaaacaccaaagtctcctgttcgctcataaagaagtttttgggatgggagagaatccagaccatcttggggcagccaggcccttgccttcatttttacagaggtagcacaactgattccaacacaaaaccccttcccctttttaaaatgatttctgttctaatgccatagatcaaaggcctcagaaaccattgtgtgtttcctctttgaagcaatgacaagcactttactttcacggtggtttttgttttttcttattgctgtggaacctcttttggaggacgttaaaggcgtgttttacttgtttttttaagagtgtgtgatgtgtgttttgtagatttcttgacagtgctgtaatacagacggcaatgcaatagcctatttaaagacactacgtgatctgattgagatgtacatagtttttttttttaccataactgaattattttatctcttatgttaacatgagaaatgtatgccaaatgattagttgatgtatgttttttaatttaatatttaaataaaatatttggaaggaaaaaa

(19) United States
(12) Patent Application Publication
(10) Pub. No.: US 2009/0136957 A1
(43) Pub. Date: May 28, 2009

(54) Methods and compositions for regulating cell cycle progression via the miR-106B family

(76) Inventors: Irena Ivanovska, Seattle, WA (US); Michael O. Carleton, Bothell, WA (US); Aimee L. Jackson, Carlsbad, CA (US); Michele A. Cleary, Bothell, WA (US); Peter S. Linsley, Encinitas, CA (US)

Correspondence Address: Eileen S. Sun;Merck & Co., Inc., P.O. Box 2000 - Patent Department RY60-30, Rahway, NJ, 07065-0907, US

(21) Appl. No.: 12/283,903

(22) Filed: Sep. 15, 2008

Related U.S. Application Data
(60) Provisional application No. 60/993,737, filed on Sep. 15, 2007.
(60) Provisional application No. 61/005,322, filed on Dec. 3, 2007.

(51) Int. Cl. C12Q 1/68; C12N 5/06

(52) U.S. Cl. 435/6; 435/375

(57) ABSTRACT

In one aspect, a method is provided of inhibiting proliferation of a mammalian cell comprising introducing into said cell an effective amount of at least one microRNA-specific inhibitor of at least one miR-106b family member. In another aspect a method is provided for accelerating proliferation of a mammalian cell comprising introducing into said cell an effective amount of at least one miR-106b family member.

This application claims priority to U.S. Provisional Patent Application Ser. No. 60/993,737 filed on Sep. 15, 2007, and U.S. Provisional Patent Application Ser. No. 61/005,322 filed on Dec. 3, 2007, each of which is incorporated by reference herein in its entirety.

This application includes a Sequence Listing submitted on compact disc, recorded on three compact discs, including one duplicate and a computer readable copy, containing Filename RS0230Y.txt, of size 69,632 bytes, created Sep. 12, 2008. The sequence listing on the compact discs is incorporated by reference herein in its entirety.

### BACKGROUND OF THE INVENTION

The following is a discussion of relevant art pertaining to miRNAs and p21. The discussion is provided only for understanding of the various embodiments of invention that follow. The summary and references cited throughout the specification herein are not an admission that any of the content below is prior art to the claimed invention.

miRNAs play important roles in diverse biological systems and miRNA mis-regulation contributes to development of disease. Our understanding of miRNA function is based primarily on determining their gene targets and, to a lesser extent, the phenotypes of miRNA overexpression and knockdown. In the context of cancer, miRNAs act either as tumor suppressors or as oncogenes. The tumor suppressor activity of the let-7 family of miRNA stems from its repression of the oncogenes Ras and HMGA2 (Lee and Dutta, 2007, Genes Dev. 21:1025-30; Mayr et al., 2007, Science 315:1576-9). The miR-16 family has an anti-proliferative effect by targeting transcripts that negatively regulate cell cycle progression, and induces apoptosis by repressing the anti-apoptotic gene BCL2 (Cimmino et al., 2005, Proc. Natl. Acad. Sci. USA 102:13944-9; Linsley et al, 2007, Mol. Cell. Biol. 27:2240-52). miR-34a is upregulated by DNA damage via the TP53 tumor suppressor and causes a cell cycle block by downregulating genes involved in cell cycle progression (Chang et al., 2007, Mol. Cell. 26:745-52; He et al, 2007, Nature 447:1130-4; Raver-Shapira et al., 2007, Mol. Cell. 26:731-43; Tarasov et al., 2007, Cell Cycle 6:1586-93). The tumor-suppressor microRNAs are often deleted or downregulated in cancers. Thus, their reintroduction may prove a viable therapeutic strategy.

Conversely, miRNAs with oncogenic properties are overexpressed or amplified in cancer and have been shown to drive tumor progression in mouse models. Individual miRNAs with oncogenic properties include miR-21, miR-155/BIC, miR-372 and miR-373 (E is et al., 2005, Proc. Natl. Acad. Sci. USA 102:3627-32; Kluiver et al., 2005, J. Pathol. 207:243-9; Si et al., 2007 Oncogene 26:2799-803; Voorhoeve et al., 2006, Cell 124:1169-81; Zhu et al., 2007, J. Biol. Chem. 282:14328-36). Several miRNA clusters show potent oncogenic characteristics. The miR-17-92 cluster is located in a region of chromosome 13 that is amplified in B cell lymphomas and ectopic expression of this cluster was shown to accelerate tumor growth in a mouse model (He et al., 2005, Nature 435:828-33). The miR-106a-363 cluster on chromosome X was identified as a site of retroviral insertion in a mouse T-cell lymphoma (Landais et al, 2007, Cancer Res. 67:5699-707). These clusters contain multiple members of a microRNA family referred here as the miR-106b family with seed-region homology, suggesting that they promote tumor growth through related, though poorly-understood cellular mechanisms.

To date, over 500 microRNAs have been described in humans, however, the current state of knowledge regarding microRNA targets and the determination of microRNA functions is incomplete. Although thousands of miRNA targets have been predicted using computational methods, relatively few predications have been experimentally validated. Computational methods are not optimal for predicting miRNA target sites. Bioinformatics approaches generally rely heavily on the detection of seed region (encompassing the first 10 bases of the mature miRNA sequence) complementary motifs that are conserved in the 3′ UTR sequences of genes across divergent species (see, e.g., John, B. et al., PloS Biol 2(11):e363, 2004). Therefore, such methods are not predictive for microRNA targets sites that are not conserved across species, or for identifying target sites that are not perfectly matched with seed regions. Moreover, target prediction using different computational methods often do not agree. Since relatively few predicted microRNA: target interactions have been experimentally confirmed, it is difficult to know how accurate such predictions are. Available methods for validation are laborious and not easily amenable to high-throughput methodologies (see e.g., Bentwich, I., FEBS Lett 579:5904-5910 (2005)).

It is important to assign functions to miRNAs and to accurately identify miRNA responsive targets. Since a single miRNA can regulate hundreds of targets, understanding of biological pathways regulated by microRNAs is not obvious from examination of their targets. As functions are assigned to miRNAs, it is also important to determine which of their target(s) are responsible for a phenotype. It is also currently unknown whether the numerous miRNA responsive targets act individually or in concert.

### SUMMARY OF THE INVENTION

In accordance with the foregoing, in one aspect, the present invention provides a method of inhibiting proliferation of a cell comprising introducing into said cell an effective amount of a miR-specific inhibitor of at least one miR-106b family member. In some embodiments, the method comprises a method of inhibiting proliferation of a mammalian cell. In a particular embodiment, said cell is a cancer cell.

In some embodiments, the at least one miR-106b family member is selected from the group consisting of: miR-106b (SEQ ID NO:1), miR-106a (SEQ ID NO:2), miR-20a (SEQ ID NO:3), miR-20b (SEQ ID NO:4), miR-17-5-p (SEQ ID NO:5). In other embodiments, the at least one miR-106b family member is selected from the group consisting of: miR-106b (SEQ ID NO:1), miR-106a (SEQ ID NO:2), miR-20a (SEQ ID NO:3), miR-20b (SEQ ID NO:4), miR-17-5-p (SEQ ID NO:5), miR-372_{—}2 (SEQ ID NO:6), and miR-93_{—}2 (SEQ ID NO:7). In one particular embodiment, the at least one miR-106b family member comprises miR-106b (SEQ ID NO:1).

Another aspect of the invention provides a method for increasing p21 function of a mammalian cell comprising introducing into said mammalian cell an effective amount of a miR-specific inhibitor of at least one miR-106b family member into the mammalian cell. In some embodiments, the at least one miR-106b family member is selected from the group consisting of: miR-106b (SEQ ID NO:1), miR-106a (SEQ ID NO:2), miR-20a (SEQ ID NO:3), miR-20b (SEQ ID NO:4), miR-17-5-p (SEQ ID NO:5). In other embodiments, the at least one miR-106b family member is selected from the group consisting of: miR-106b (SEQ ID NO:1), miR-106a (SEQ ID NO:2), miR-20a (SEQ ID NO:3), miR-20b (SEQ ID NO:4), miR-17-5-p (SEQ ID NO:5), miR-372_{—}2 (SEQ ID NO:6), and miR-93-2 (SEQ ID NO:7). In one particular embodiment, the at least one miR-106b family member comprises miR-106b (SEQ ID NO:1).

In some embodiments, the miR-specific inhibitor may be an anti-miR, antagomir, microRNA sponge, and target mimics. In a particular embodiment, the miR-specific inhibitor comprises a polynucleic acid molecule comprising a nucleotide sequence of at least six contiguous nucleotides that is complementary to positions 2-8 of the miR-106b seed region (“AAAGUGC” SEQ ID NO:8).

Another aspect of the invention provides a method of accelerating proliferation of a cell comprising introducing an effective amount of a small interfering nucleic acid (siNA) into the cell, wherein said siNA comprises a guide strand of contiguous nucleotide sequence of at least 18 nucleotides, wherein said guide strand comprises a seed region consisting of nucleotides positions 1 to 10, wherein position 1 represents the 5′ end of said guide strand and wherein said seed region comprises a nucleotide sequence of at least 6 contiguous nucleotides that are identical to the miR-106b seed region (“AAAGUGC” SEQ ID NO:8). In another embodiment, said effective amount of a small interfering nucleic acid comprises miR-106b (SEQ ID NO:1), miR-106a (SEQ ID NO: 2), miR-20a (SEQ ID NO:3), miR-20b (SEQ ID NO:4), or miR-17-5-p (SEQ ID NO:5).

Another aspect of the invention provides a method for decreasing p21 function of a mammalian cell comprising introducing into said mammalian cell an effective amount of a small interfering nucleic acid (siNA) into the cell, wherein said siNA comprises a guide strand contiguous nucleotide sequence of at least 18 nucleotides, wherein said guide strand comprises a seed region consisting of nucleotides positions 1 to 10, wherein position 1 represents the 5′ end of said guide strand and wherein said seed region comprises a nucleotide sequence of at least 6 contiguous nucleotides that are identical to the miR-106b seed region (“AAAGUGC” SEQ ID NO:8). In another embodiment, said effective amount of a small interfering nucleic acid comprises miR-106b (SEQ ID NO:1), miR-106a (SEQ ID NO: 2), miR-20a (SEQ ID NO:3), miR-20b (SEQ ID NO: 4), or miR-17-5-p (SEQ ID NO:5).

Alternatively, the invention provides a method for decreasing LIMK1, NKIRAS1, MAPRE3, RNH1, or MAPK1 of a mammalian cell comprising introducing into said mammalian cell an effective amount of a small interfering nucleic acid (siNA) into the cell, wherein said siNA comprises a guide strand contiguous nucleotide sequence of at least 18 nucleotides, wherein said guide strand comprises a seed region consisting of nucleotides positions 1 to 10, wherein position 1 represents the 5′ end of said guide strand and wherein said seed region comprises a nucleotide sequence of at least 6 contiguous nucleotides that are identical to the miR-106b seed region (“AAAGUGC” SEQ ID NO:8). In another embodiment, said effective amount of a small interfering nucleic acid comprises miR-106b (SEQ ID NO:1), miR-106a (SEQ ID NO: 2), miR-20a (SEQ ID NO:3), miR-20b (SEQ ID NO: 4), or miR-17-5-p (SEQ ID NO:5).

In another embodiment, the invention provides a method for increasing LIMK1, NKIRAS1, MAPRE3, RNH1, or MAPK1 function of a mammalian cell comprising introducing into said mammalian cell an effective amount of miR-specific inhibitor of at least one miR-106b family member into the mammalian cell. In some embodiments, the at least one miR-106b family member is selected from the group consisting of: miR-106b (SEQ ID NO:1), miR-106a (SEQ ID NO:2), miR-20a (SEQ ID NO:3), miR-20b (SEQ ID NO:4), miR-17-5-p (SEQ ID NO:5). In other embodiments, the at least one miR-106b family member is selected from the group consisting of: miR-106b (SEQ ID NO:1), miR-106a (SEQ ID NO:2), miR-20a (SEQ ID NO:3), miR-20b (SEQ ID NO:4), miR-17-5-p (SEQ ID NO:5), miR-372_{—}2 (SEQ ID NO:6), and miR-93_{—}2 (SEQ ID NO:7). In one particular embodiment, the at least one miR-106b family member comprises miR-106b (SEQ ID NO:1).

In some embodiments, the miR-specific inhibitor may be an anti-miR, antagomir, microRNA sponge, and target mimics. In a particular embodiment, the miR-specific inhibitor comprises a polynucleic acid molecule comprising a nucleotide sequence of at least six contiguous nucleotides that is complementary to the miR-106b seed region (“AAAGUGC” SEQ ID NO:8).

In some embodiments, said siNA comprises synthetic RNA duplexes. In some embodiments, the siNA further comprises a non-nucleotide moiety. In another embodiment of said method, the guide strand and a passenger strand are stabilized against nucleolytic degradation. In a more particular embodiment, said siNA comprises at least one chemically modified nucleotide or non-nucleotide at the 5′ end and/or 3′ end of the guide strand and the 3′ end of the passenger strand.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates the miR-106b family. A) miR-106b family expression levels are positively correlated with cell-cycle functional annotation in a human tumor atlas. miRNA levels measured in human tumor samples were correlated with mRNA levels in the same samples. Sets of >100 transcripts positively correlated (r>0.4) with a miRNA were annotated with G0 Biological Process terms. Shown is a heat map depicting 2D cluster of E-value enrichment for the most common G0 Biological Process terms (X-axis) associated with microRNAs (Y-axis). The E-value is a conservative adjustment of the P-value to take into account that multiple sets were tested. To calculate the E-values, the p-value was Bonferroni corrected. B) Sequence alignment of the miR-106b family. This is one of the largest family of microRNAs with 18 members (not shown are 4 miR-520 and 4 miR-519 variants). *'s denote positions 2-8 of the seed region. miR-106b, miR-106a, miR-20b, miR-20a, and miR-17-5p share seed identity and phenotypes. miR-93, miR-372, miR-520, miR-526* and miR-519 have seed regions that are off-set by one base at the 5′ end and miR-18 has a seed region with one divergent base at position 4 (underlined). C) The miR-106b family is overexpressed in tumors. microRNA levels in tumor and adjacent normal tissues from a tumor atlas were measured (Raymond et al, 2005, RNA 11:1737-44). Shown are log 2 values for ratios of miR-106b family levels in tumor and normal samples to average levels of the same microRNAs in the corresponding normal tissues (normal pool).

FIG. 2. Gain-of-function of the miR-106b family accelerates cell cycle progression; knockdown decelerate the cell cycle. (A1) miR-106b promotes cell division. A growth curve measuring cell numbers following transfection of a control duplex, miR-106b, or anti-miR-106b into HMECs shows that miR106b promotes, whereas anti-miR-106b retards cell division. (A2) miR-106b and miR-106a gain-of-function led to an increase in S-phase cells. Human mammary epithelial cells (HMECs) were transfected with the indicated microRNA or control duplex (luciferase). BrdU incorporation was analyzed using flow cytometry. Shown are scatter plots of fluorescence intensities of BrdU incorporation (Y-axis) against DNA content (X-axis). Gates capture the S-phase populations (positive for BrdU incorporation) and the numbers depict percent of cells in S phase. (B) miR-106b family gain-of-function accelerates G1-to-S progression; anti-miRs retard this cell cycle transition. HMECs were transfected with control duplex (luciferase, top panel), microRNAs (middle panels), or anti-miRs (bottom panels) and treated with nocodazole for 16 hr. miR-106b mutant has mutations at positions 2 and 3 of the seed region (top right panel). Cell cycle profiles were analyzed using flow cytometry. Shown are histograms of cell numbers (Y-axis) against DNA content (measured by fluorescence intensity, X-axis). (C) miR-106b family levels are reduced by anti-miR-106b. HMECs were treated with anti-miRs against family members (anti-miR-106b shown) and RNA samples were profiled at the Rosetta gene expression laboratory. microRNA abundance of mock-treated cells (X-axis) was plotted against anti-miR-106b-treated cells (Y-axis).

FIG. 3. Microarray analysis identifies putative miR-106b-family targets. (A) Consensus set of genes regulated by the miR-106b family members. A heat map showing 96 of the 103 genes that are significantly downregulated by miR-106b, miR-106a, miR-17-5p, and miR-20b, representing the likely direct targets. miR-93 and miR-372 downregulate the majority of these genes whereas miR-18, miR-16, and miR-34a largely do not affect them. (B) siRNA knockdown of cell-cycle genes predicted to be downregulated by miR-106b family phenocopies miR-106b-family gain-of-function. siRNA-mediated knockdown of p21/CDKN1A, LIMK1, and NKIRAS1 leads to reduction in G1-phase cells upon treatment with nocodazole as seen for miR-106b gain-of-function.

FIG. 4. p21 mRNA and protein levels respond to miR-106b. (A.1) Transfection of several miR-106b family members down-regulates luciferase reporter/p21 3′UTR expression construct. Shown is a representative experiment for miR-106b and miR-106a transfection. Mock transfection, miR-18 and miR-34a do not affect the reporter, as expected. p21 siRNAs against the 3′UTR are used as a positive control. (A.2) Several miR-106b family members modulate a p21-3′UTR reporter plasmid. The luciferase open reading frame was fused to the entire p21 3′UTR. Co-transfection of this construct with miR-106b family duplexes resulted in down-regulation of luciferase activity (gray bars), as compared to a construct in which the miR-106b-seed-region complementarity sites were mutated (black bars). (B) p21 mRNA levels were reduced by miR-106b gain of function. HMECs were treated with miR-106b or a luciferase control and p21 mRNA levels were measured by TaqMan. Shown are relative levels normalized against hGUS (a housekeeping gene). (C) p21 protein levels were reduced by miR-106b overexpression and increased by anti-miR-106b. HMECs were treated with miR-106b, anti-miR-106b or a luciferase control and p21 protein levels were measured by immunoblotting. Shown are relative levels normalized against HSP70 (a housekeeping protein). (D) p21 is required for the anti-miR-106b phenotype. HMECs were transfected with control duplex (luciferase), anti-miR-106b, p21 siRNAs, or anti-miR106b and p21 siRNAs and were treated with nocodazole for 24 hours. Cell cycle profiles were analyzed using flow cytometry. Shown are histograms of cell numbers (Y-axis) against DNA content (measured by fluorescence intensity, X-axis). Numbers denoted the percent of cells in G1.

FIG. 5. miR-106b gain-of-function and p21 knockdown share common phenotypes and

p21 is required for anti-miR-106b to slow cell cycle progression. (A) p21 knockdown results in increased S-phase population. HMECs were transfected with control duplex (luciferase, left panel), miR-106b (middle panel) or p21 siRNAs (right panel). BrdU incorporation was analyzed using flow cytometry. Shown are scatter plots of fluorescence intensities of BrdU incorporation (Y-axis) against DNA content (X-axis). Gray gates capture the S-phase populations (positive for BrdU incorporation) and the numbers depict percent of cells in S phase. (B) miR-106b overrides the TP53/p21-dependent G1 arrest upon DNA damage. HMECs were transfected with control duplex (luciferase), miR-106b, or p21 siRNAs and were treated with Doxorubicin for 48 hr. Cell cycle profiles were analyzed using flow cytometry. Shown are histograms of cell numbers (Y-axis) against DNA content (measured by fluorescence intensity, X-axis). Numbers denote the percent of cells in G1 and G2/M, respectively. (C) miR-106b and p21 loss promote endoreduplication in Nocodazole-blocked cells. HMECs were transfected with control duplex (luciferase), miR-106b, p21 siRNAs or miR-106b and p21 siRNAs and were treated with Nocodazole for 48 hr. Cell cycle profiles were analyzed using flow cytometry. Shown are histograms of cell numbers (Y-axis) against DNA content (measured by fluorescence intensity, X-axis). Numbers denote the percent of cells with 8N DNA content. (D) Loss of anti-miR-106b effect upon p21 knockdown. HMECs were transfected with control duplex (luciferase), anti-miR-106b, p21 siRNAs or anti-miR-106b and p21 siRNAs and were treated with nocodazole for 24 h. Cell cycle profiles were analyzed using flow cytometry. Shown are histograms of cell numbers (Y-axis) against DNA content (measured by fluorescence intensity, X-axis). Numbers denote the percent of cells in G1.

FIG. 6. miR-93 and miR-372 have subtle effects on cell cycle progression. (A) During the course of this study, an alternative miR-93 was cloned {Landgraf et al., 2007, Cell 129:1401-1414} (referred to here as miR-93_{—}2) containing an additional base at the 5′ end, putting it in register with miR-106a, miR106b, miR-17-5p, and miR-20. miR-372 was not cloned in this study likely due to its low expression in somatic tissues. We tested the phenotypes of both the original sequences and of sequences containing an addition base at the 5′ end to determine the dependence of microRNA sequence on the base composition. (B) We found that the original sequences did not show the miR-106b-phenotype of lower G1 population, whereas the sequences with the additional base at the 5′ end had a more subtle effect on the G1 population that the other family members. Anti-miRs that knock down the endogenous microRNAs resulted in slower cell-cycle progression similar to the effect of anti-miRs against other family members.

FIG. 7. anti-miR-106b causes G1 block. A subpopulation of 20% of cells remain in G1 even after prolonged exposure to nocodazole.

FIG. 8. miR-106b and p21 synergize in HCT116 cells. (A) miR-106b overexpression and

p21 knockout led to an increase in S-phase population in HCT116 cells. Combined, these treatments have a synergistic effect. (B) miR-106b overexpression and p21 knockout overrode the Nocodazole-mediated G2/M block and resulted in endoreduplication and the accumulation of 8N cells in HCT116. Combined, these treatments have a synergistic effect on endoreduplication.

FIG. 9. 2′-O-methyl modified anti-miR-106b causes G1 block. 2′-O-methyl modified anti-miR-106b reverses the miR-106b family phenotype with equivalent results as the LNA-modified anti-miR-106b. HMEC H1 term cells were transfected with 10 nM control siRNA, 10 nM miR-106b or 10 nM miR-106b+200 nM 2′-O-Me anti-miR-106b and were treated with nocodazole for 18 h.

### DETAILED DESCRIPTION OF THE INVENTION

This section presents a detailed description of the many different aspects and embodiments that are representative of the inventions disclosed herein. This description is by way of several exemplary illustrations, of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure the ordinary skilled artisan can identify and employ other components and methodology useful for practicing the present invention.

### I. DEFINITIONS

Unless defined otherwise, all technical and scientific terms used herein have the meaning commonly understood by one of ordinary skill in the art to which this invention belongs. Practitioners are particularly directed to Sambrook et al. (1989) Molecular Cloning: A Laboratory Manual, 2d ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It is contemplated that any embodiment discussed in this specification can be implemented with respect to any method, kit, reagent, or composition of the invention, and vice versa. Furthermore, compositions of the invention can be used to achieve methods of the invention.

“Biomarker” means any gene, protein, or an EST derived from that gene, the expression or level of which changes between certain conditions. Biomarker may also include miRNAs. Where the expression of the gene correlates with a certain condition, the gene is a biomarker for that condition. In particular, high miR-106b expression correlates with important characteristics of tumors, i.e. acceleration of cell cycle progression (i.e. G1-to-S phase).

As used herein, the term “gene” has its meaning as understood in the art. However, it will be appreciated by those of ordinary skill in the art that the term “gene” may include gene regulatory sequences (e.g., promoters, enhancers, etc.) and/or intron sequences. It will further be appreciated that definition of gene includes references to nucleic acids that do not encode proteins but rather encode functional RNA molecules such as tRNAs or precursor miRNAs. For clarity, the term gene generally refers to a portion of a nucleic acid that encodes a protein; the term may optionally encompass regulatory sequences. This definition is not intended to exclude application of the term “gene” to non-protein coding expression units but rather to clarify that, in most cases, the term, as used in this document refers to a protein coding nucleic acid. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular embodiments, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

As used herein, the term “microRNA species”, “microRNA”, “miRNA”, or “miR” refers to small, non-protein coding RNA molecules that are expressed in a diverse array of eukaryotes, including mammals. MicroRNA molecules typically have a length in the range of from 15 to 120 nucleotides, the size depending upon the specific microRNA species and the degree of intracellular processing. Mature, fully processed miRNAs are about 15 to 30, 15-25, or 20 to 30 nucleotides in length, and more often between about 16 to 24, 17 to 23, 18 to 22, 19 to 21, or 21 to 24 nucleotides in length. MicroRNAs include processed sequences as well as corresponding long primary transcripts (pri-miRNAs) and processed precursors (pre-miRNAs). Some microRNA molecules function in living cells to regulate gene expression via RNA interference. A representative set of microRNA species is described in the publicly available miRBase sequence database as described in Griffith-Jones et al., Nucleic Acids Research 32:D109-D111 (2004) and Griffith-Jones et al., Nucleic Acids Research 34:D 140-D144 (2006), accessible on the World Wide Web at the Wellcome Trust Sanger Institute website. MicroRNAs may also include synthetic RNA duplex and vector-encoded hairpin molecules, designed to mimic the miRNAs (Lim et al., 2005, Nature, 433:769773; Linsley et al., 2007, Mol. Cell. Biol., 27:2240-2252, which are incorporated by reference herein).

As used herein, the term “microRNA family” refers to a group of microRNA species that share identity across at least 6 consecutive nucleotides within nucleotide positions 1 to 10 of the 5′ end of the microRNA molecule, also referred to as the “seed region”, as described in Brennecke, J. et al., PloS biol 3(3):e85 (2005).

As used herein, the term “microRNA family member” refers to a microRNA species that is a member of a microRNA family, including naturally occurring microRNA species and artificially generated microRNA molecules.

As used herein, the term “RNA interference” or “RNAi” refers to the silencing or decreasing of gene expression by iRNA agents (e.g., siRNAs, miRNAs, shRNAs), via the process of sequence-specific, post-transcriptional gene silencing in animals and plants, initiated by an iRNA agent that has a seed region sequence in the iRNA guide strand that is complementary to a sequence of the silenced gene or target sequence.

As used herein, the term an “iNA agent” (abbreviation for “interfering nucleic acid agent”), refers to a nucleic acid agent, for example RNA, or chemically modified RNA, which can down-regulate the expression of a target gene. While not wishing to be bound by theory, an iNA agent may act by one or more of a number of mechanisms, including post-transcriptional cleavage of a target mRNA, or pre-transcriptional or pre-translational mechanisms. An iNA agent can include a single strand (ss) or can include more than one strands, e.g., it can be a double stranded (ds) iNA agent. An iNA agent may include iRNA agents.

As used herein, the term “single strand iRNA agent” or “ssRNA agent” is an iRNA agent which consists of a single molecule. It may include a duplexed region, formed by intra-strand pairing, e.g., it may be, or include, a hairpin or panhandle structure. The ssRNA agents of the present invention include transcripts that adopt stem-loop structures, such as shRNA, that are processed into a double stranded siRNA.

As used herein, the term “ds iNA agent” is a dsNA (double stranded nucleic acid (NA)) agent that includes two strands that are not covalently linked, in which interchain hybridization can form a region of duplex structure. The dsNA agents of the present invention include silencing dsNA molecules that are sufficiently short that they do not trigger the interferon response in mammalian cells.

As used herein, the term “siRNA” refers to a small interfering RNA. siRNA include short interfering RNA of about 15-60, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-40, 15-30, 15-25 or 19-25 nucleotides in length, preferably about 20-24 or about 21-22 or 21-23 nucleotides in length, preferably 19-21 nucleotides in length, and the double stranded siRNA is about 15-60, 15-50, 1540, 15-30, 15-25 or 19-25 preferably about 20-24 or about 21-22 or 19-21 or 21-23 base pairs in length). siRNA duplexes may further comprise 3′ overhangs of about 1 to about 4 nucleotides, preferably of about 2 to about 3 nucleotides and a 5′ phosphate termini. In some embodiments, the siRNA lacks a terminal phosphate. In some embodiments, one or both ends of siRNAs can include single-stranded 3′ overhangs that are two or three nucleotides in length, such as, for example, deoxythymidine (dTdT) or uracil (UU) that are not complementary to the target sequence. In some embodiments, siRNA molecules can include nucleotide analogs (e.g. phosphorothioate, phosphonoacetate, or thiophosphonoacetate) and other modifications useful for enhanced nuclease resistance, enhanced duplex stability, enhanced cellular uptake, or cell targeting.

In certain embodiments, at least one of the two strands of the siRNA further comprises a 1-4, preferably a 2 nucleotide overhang. The nucleotide overhang can include any combination of a thymine, uracil, adenine, guanine, or cytosine, or derivatives or analogues thereof. The nucleotide overhang in certain aspects is a 2 nucleotide overhang, where both nucleotides are thymine. Importantly, when the dsRNA comprising the sense and antisense strands is administered, it directs target specific interference and bypasses an interferon response pathway. In order to enhance the stability of the short interfering nucleic acids, the 3′ overhangs can also be stabilized against degradation. In one embodiment, the 3′ overhangs are stabilized by including purine nucleotides, such as adenosine or guanosine nucleotides. Alternatively, substitution of pyrimidine nucleotides by modified analogues, e.g. substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

As used herein, a “3′ overhang” refers to at least one unpaired nucleotide extending from the 3′ end of an siRNA sequence. The 3′ overhang can include ribonucleotides or deoxyribonucleotides or modified ribonucleotides or modified deoxyribonucleotides. The 3′ overhang is preferably from 1 to about 4 nucleotides in length, more preferably from 1 to about 4 nucleotides in length and most preferably from about 2 to about 4 nucleotides in length. The 3′ overhang can occur on the sense or antisense sequence, or on both sequences of an RNAi construct. The length of the overhangs can be the same or different for each strand of the duplex. Most preferably, a 3′ overhang is present on both strands of the duplex, and the overhang for each strand is 2 nucleotides in length. For example, each strand of the duplex can comprise 3′ overhangs of dithymidylic acid (“tt”) or diuridylic acid (“uu”).

Non limiting examples of siRNA molecules of the invention may include a double-stranded polynucleotide molecule comprising self-complementary sense and antisense regions, wherein the antisense region comprises nucleotide sequence that is complementary to nucleotide sequence in a target nucleic acid molecule or a portion thereof (alternatively referred to as the guide region, or guide strand when the molecule contains two separate strands) and the sense region having nucleotide sequence corresponding to the target nucleic acid sequence or a portion thereof (also referred as the passenger region, or the passenger strand when the molecule contains two separate strands). The siRNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 18 to about 30, e.g., about 18, 19, 20, 21, 22, 23, 24, 25, 26, 27, 28, 29 or 30 base pairs); the antisense strand (guide strand) comprises nucleotide sequence that is complementary to nucleotide sequence in a target nucleic acid molecule or a portion thereof and the sense strand (passenger strand) comprises nucleotide sequence corresponding to the target nucleic acid sequence or a portion thereof (e.g., about 15 to about 25 nucleotides of the siRNA molecule are complementary to the target nucleic acid or a portion thereof). Typically, a short interfering RNA (siRNA) refers to a double-stranded RNA molecule of about 17 to about 29 base pairs in length, preferably from 19-21 base pairs, one strand of which is complementary to a target mRNA, that when added to a cell having the target mRNA or produced in the cell in vivo, causes degradation of the target mRNA. Preferably the siRNA is perfectly complementary to the target mRNA. But it may have one or two mismatched base pairs.

Alternatively, the siRNA is assembled from a single oligonucleotide, where the self-complementary sense and antisense regions of the siRNA are linked by means of a nucleic acid based or non-nucleic acid-based linker(s). The siRNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having a nucleotide sequence corresponding to the target nucleic acid sequence or a portion thereof. The siRNA can be a circular single-stranded polynucleotide having two or more loop structures and a stem comprising self-complementary sense and antisense regions, wherein the antisense region comprises a nucleotide sequence that is complementary to a nucleotide sequence in a target nucleic acid molecule or a portion thereof and the sense region comprises a nucleotide sequence corresponding to the target nucleic acid sequence or a portion thereof, and wherein the circular polynucleotide can be processed either in vivo or in vitro to generate an active siRNA molecule capable of mediating RNAi. The siRNA can also comprise a single stranded polynucleotide having nucleotide sequence complementary to a nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell 110:563-574 and Schwarz et al., 2002, Molecular Cell, 10:537-568), or 5′,3′-diphosphate. In certain embodiments, the siRNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In another embodiment, the siRNA molecule of the invention interacts with nucleotide sequence of a target gene in a manner that causes inhibition of expression of the target gene.

As used herein, the siRNA molecules need not be limited to those molecules containing only RNA, but may further encompass chemically modified nucleotides and non-nucleotides. WO2005/078097; WO2005/0020521; and WO2003/070918 detail various chemical modifications to RNAi molecules, and the contents of each reference is hereby incorporated by reference in its entirety. In certain embodiments, for example, the siRNA molecules may lack 2′-hydroxyl (2′-OH) containing nucleotides. The siRNA can be chemically synthesized or may be encoded by a plasmid (e.g. transcribed as sequences that automatically fold into duplexes with hairpin loops). siRNA can also be generated by cleavage of longer dsRNA (e.g. dsRNA greater than about 25 nucleotides in length) with the E. coli RNAse III or Dicer. These enzymes process the dsRNA into biologically active siRNA (see, e.g. Yang et al., 2002, Proc. Natl. Acad. Sci. USA 99:9942-7; Calegari et al., 2002, Proc. Natl. Acad. Sci. USA 99:14236; Bryrom et al., 2003, Ambion TechNotes 10(1):4-6; Kawasaki et al., 2003, Nucleic Acids Res. 31:981-7; Knight and Bass, 2001, Science 293:2269-71; and Robertson et al., 1968, J. Biol. Chem. 243:81). The long dsRNA can encode for an entire gene transcript or a partial gene transcript.

As used herein, “percent modification” refers to the number of nucleotides in each of the strand of the siRNA or to the collective dsRNA that have been modified. Thus 19% modification of the antisense strand refers to the modification of up to 4 nucleotides/bp in a 21 nucleotide sequence (21 mer). 100% refers to a fully modified dsRNA. The extent of chemical modification will depend upon various factors well known to one skilled in the art. Such, as for example, target mRNA, off-target silencing, degree of endonuclease degradation, etc.

As used herein, the term “shRNA” or “short hairpin RNAs” refers to an RNA molecule that forms a stem-loop structure in physiological conditions, with a double-stranded stem of about 17 to about 29 base pairs in length, where one strand of the base-paired stem is complementary to the mRNA of a target gene. The loop of the shRNA stem-loop structure may be any suitable length that allows inactivation of the target gene in vivo. While the loop may be from 3 to 30 nucleotides in length, typically it is 1-10 nucleotides in length. The base paired stem may be perfectly base paired or may have 1 or 2 mismatched base pairs. The duplex portion may, but typically does not, contain one or more bulges consisting of one or more unpaired nucleotides. The shRNA may have non-base-paired 5′ and 3′ sequences extending from the base-paired stem. Typically, however, there is no 5′ extension. The first nucleotide of the shRNA at the 5′ end is a G, because this is the first nucleotide transcribed by polymerase III. If G is not present as the first base in the target sequence, a G may be added before the specific target sequence. The 5′ G typically forms a portion of the base-paired stem. Typically, the 3′ end of the shRNA is a poly U segment that is a transcription termination signal and does not form a base-paired structure. As described in the application and known to one skilled in the art, shRNAs are processed into siRNAs by the conserved cellular RNAi machinery. Thus, shRNAs may be precursors of siRNAs and are, in general, similarly capable of inhibiting expression of a target mRNA transcript. For the purpose of description, in certain embodiments, the shRNA constructs of the invention target one or more mRNAs that are targeted by miR-106b, miR-106a, miR-20b, miR-20a, or miR-17-5p. The strand of the shRNA that is antisense to the target gene transcript is also known as the “guide strand”.

As used herein, the term “microRNA responsive target site” or “microRNA binding site” refers to a nucleic acid sequence ranging in size from about 5 to about 25 nucleotides (such as 5, 6, 7, 8, 9, 10, 11, 12, 13, 14, 15, 16, 17, 18, 19, 20, 21, 22, 23, 24, or 25 nucleotides) that is complementary, or essentially complementary to at least a portion of a microRNA molecule. In some embodiments, the microRNA responsive target site comprises at least 5 consecutive nucleotides, at least 6 consecutive nucleotides, at least 7 consecutive nucleotides, at least 8 consecutive nucleotides, or at least 9 nucleotides that are complementary to the seed region of a microRNA molecule (i.e., within nucleotide positions 1 to 10 of the 5′ end of the microRNA molecule, referred to as the “seed region”. See, e.g. Brennecke et al., 2005, PLOS Biol. 3(3):e85. In some embodiments, the microRNA responsive target site comprises at least 6 consecutive nucleotides that are complementary to positions 2-8 of the seed region of a microRNA molecule.

As used herein, the term “miR-specific inhibitor” refers to a nucleic acid molecule that is complementary, or essentially complementary to at least a portion of a microRNA molecule and inhibits its binding or activity towards its target gene transcripts. A miR-specific inhibitor may interact with the miRNA directly or may interact with the miRNA binding site in a target transcript, preventing its interaction with a miRNA. In some embodiments, the miR-specific inhibitor comprises a nucleotide sequence of at least 5 consecutive nucleotides, at least 6 consecutive nucleotides, at least 7 consecutive nucleotides, at least 8 consecutive nucleotides, or at least 9 nucleotides that are complementary to the seed region of a microRNA molecule (i.e. within positions 1 to 10 of the 5′ end of the microRNA molecule referred to as the “seed region”). In a particular embodiment, the miR-specific inhibitor may comprise a nucleotide sequence of at least 6 consecutive nucleotides that are complementary to the seed region of a microRNA molecule at positions 2-8. These consecutive nucleotides complementary to the microRNA seed region may also be referred to as microRNA binding sites. A miR-specific inhibitor may be a single stranded molecule. The miR-specific inhibitor may be chemically synthesized or may be encoded by a plasmid. In some embodiments, the miR-specific inhibitor comprises RNA. In other embodiments, the miR-specific inhibitor comprises DNA. In other embodiments, the miR-specific inhibitor may encompass chemically modified nucleotides and non-nucleotides. See, e.g. Brennecke et al., 2005, PLOS Biol. 3(3):pe85.

In some embodiments, a miR-specific inhibitor may be an anti-miRNA (anti-miR) oligonucleotide (see WO2005054494; Hutvagner et al., 2004, PLoS Biol. 2:E98; Orom et al., 2006, Gene 372:137-141;). Anti-miRs may be single stranded molecules. Anti-miRs may comprise RNA or DNA or have non-nucleotide components. Alternative embodiments of anti-miRs may be as described above for miR-specific inhibitors. Anti-miRs anneal with and block mature microRNAs through extensive sequence complementarity. In some embodiments, an anti-miR may comprise a nucleotide sequence that is a perfect complement of the entire miRNA. In some embodiments, an anti-miR comprises a nucleotide sequence of at least 6 consecutive nucleotides that are complementary to the seed region of a microRNA molecule at positions 2-8 and has at least 50%, 60%, 70%, 80%, or 90% complementarity to the rest of the miRNA. In other embodiments, the anti-miR may comprise additional flanking sequence, complimentary to adjacent primary (pri-miRNA) sequences. Chemical modifications, such as 2′-O-methyl; LNA; and 2′-O-methyl, phosphorothioate, cholesterol (antagomir); 2′-O-methoxyethyl have been described for anti-miRs (WO2005054494; Hutvagner et al., 2004, PLoS Biol. 2:e98; Meister et al., 2004, RNA 10:544-50; Orom et al., 2006, Gene 372:137-41; WO2005079397; Krutzfeldt et al., 2005, Nature 438:685-689; Davis et al, 2006; Nucleic Acid Res. 34:2294-2304; Esau et al., 2006, Cell Metab. 3:87-98). Chemically modified anti-miRs are commercially available from a variety of sources, including but not limited to Sigma-Proligo, Ambion, Exiqon, and Dharmacon.

For example, an anti-miR-106b may comprise a single stranded molecule comprising a sequence that is a perfect complement of miR-106b (SEQ ID NO:1) (i.e. the guide strand sequence is “AUCUGCACUGUCAGCACUUUA” SEQ ID NO:9). Other exemplary anti-miRs comprising a nucleotide sequence that is a perfect complement of the target miRNA are shown below.

| | | |
|---|---|---|
| Anti-miR | Sequences | SEQ ID NO: |

| | | |
|---|---|---|
| Anti-miR-106b | AUCUGCACUGUCAGCACUUUA | SEQ ID NO:9 |
| Anti-miR-106a | GCUACCUGCACUGUAAGCACUUUU | SEQ ID NO:10 |
| Anti-miR-20a | CUACCUGCACUAUAAGCACUUUA | SEQ ID NO:11 |
| Anti-miR-20b | CUACCUGCACUAUAAGCACUUUG | SEQ ID NO:12 |
| Anti-miR-17- | ACUACCUGCACUGUAAGCACUUUG | SEQ ID NO:13 |
| 5-p | | |
| Anti-miR-93 | CUACCUGCACGAACAGCACUUU | SEQ ID NO:14 |
| Anti-miR- | CUACCUGCACGAACAGCACUUUG | SEQ ID NO:15 |
| 93_2 | | |
| Anti-miR-372 | ACGCUCAAAUGUCGCAGCACUUU | SEQ ID NO:16 |
| Anti-miR- | ACGCUCAAAUGUCGCAGCACUUUC | SEQ ID NO:17 |
| 372_2 | | |

In a specific embodiment, a miR-106b-family-specific inhibitor targets at least one miR-106b-family member and comprises a nucleotide sequence of at least 6 consecutive nucleotides that are complementary to positions 2-8 of the seed region of the miR-106b family member and has at least 50%, 60%, 70%, 80% or 90% complementarity to the rest of the miR-106b family members, wherein the miR-106b-family specific inhibitor retards the G1-S transition. Alternatively, the miR-106b-family-specific inhibitor up-regulates p21.

In some embodiments, miR-specific inhibitors possess at least one microRNA binding site, mimicking the microRNA target (target mimics). These target mimics may possess at least one nucleotide sequence comprising 6 consecutive nucleotides complementary to positions 2-8 of the miRNA seed region. Alternatively, these target mimics may comprise a nucleotide sequence with complementarity to the entire miRNA. These target mimics may be vector encoded. Vector encoded target mimics may have one or more microRNA binding sites in the 5′ or 3′ UTR of a reporter gene. The target mimics may possess microRNA binding sites for more than one microRNA family. The microRNA binding site of the target mimic may be designed to mismatch positions 9-12 of the microRNA to prevent miRNA-guided cleavage of the target mimic. Target mimics have been previously described (Franco-Zorrilla et al, 2007, Nature Genet. 39:1033-1037; Ebert et al., 2007, Nature Methods 4:721-6).

In an alternative embodiment, a miR-specific inhibitor may interact with the miRNA binding site in a target transcript, preventing its interaction with a miRNA. Target protector morpholino antisense oligonucleotides protect the target transcript from the miRNA (Choi et al., Aug. 30, 2007, Sciencexpress advance online publication, 10.1126/science.1147535). These target protector morpholino oligos comprise a nucleotide sequence of at least 6 consecutive nucleotides with 100% complementarity to the miRNA binding sequence (corresponding to positions 2-8 of the miRNA seed region) and additional sequences complementary to the 3′ UTR of the target mRNA transcript. The additional sequences complementary to the 3′ UTR of the target mRNA transcript may or may not have identity with the corresponding miRNA.

The phrase “inhibiting expression of a target gene” refers to the ability of an RNAi agent, such as an siRNA, to silence, reduce, or inhibit expression of a target gene. Said another way, to “inhibit”, “down-regulate”, or “reduce”, it is meant that the expression of the gene, or level of RNA molecules or equivalent RNA molecules encoding one or more proteins or protein subunits, or activity of one or more proteins or protein subunits, is reduced below that observed in the absence of the RNAi agent. For example, an embodiment of the invention proposes inhibiting, down-regulating or reducing expression of one or more p21 pathway genes, by introduction of a miR-106b-like siRNA molecule, below the level observed for that p21 pathway gene in a control cell to which a miR-106b-like siRNA molecule has not been introduced. In another embodiment, inhibition, down-regulation, or reduction contemplates inhibition of the target mRNA below the level observed in the presence of, for example, an siRNA molecule with scrambled sequence or with mismatches. In yet another embodiment, inhibition, down-regulation, or reduction of gene expression with an siRNA molecule of the instant invention is greater in the presence of the inventive siRNA e.g., siRNA that down regulates one or more p21 pathway gene mRNAs levels than in its absence. In one embodiment, inhibition, down regulation, or reduction of gene expression is associated with post transcriptional silencing, such as RNAi mediated cleavage of a target nucleic acid molecule (e.g. RNA) or inhibition of translation.

To examine the extent of gene silencing, a test sample (e.g., a biological sample from organism of interest expressing the target gene(s) or a sample of cells in culture expressing the target gene(s)) is contacted with an siRNA that silences, reduces, or inhibits expression of the target gene(s). Expression of the target gene in the test sample is compared to expression of the target gene in a control sample (e.g., a biological sample from organism of interest expressing the target gene or a sample of cells in culture expressing the target gene) that is not contacted with the siRNA. Control samples (i.e., samples expressing the target gene) are assigned a value of 100%. Silencing, inhibition, or reduction of expression of a target gene is achieved when the value of the test sample relative to the control sample is about 95%, 90%, 85%, 80%, 75%, 70%, 65%, 60%, 55%, 50%, 45%, 40%, 35%, 30%, 25%, 20%, 10% or 0%. Suitable assays include, e.g., examination of protein or mRNA levels using techniques known to those of skill in the art such as dot blots, northern blots, in situ hybridization, ELISA, microarray hybridization, immunoprecipitation, enzyme function, as well as phenotypic assays known to those of skill in the art.

An “effective amount” or “therapeutically effective amount” of an siRNA, RNAi agent, or miR-specific inhibitor is an amount sufficient to produce the desired effect, e.g., inhibition of expression of a target sequence in comparison to the normal expression level detected in the absence of the siRNA RNAi agent, or miR-specific inhibitor. Inhibition of expression of a target gene or target sequence by a siRNA, RNAi agent, or miR-specific inhibitor is achieved when the expression level of the target gene mRNA or protein is about 90%, 80%, 70%, 60%, 50%, 40%, 30%, 25%, 20%, 15%, 10%, 5%, or 0% relative to the expression level of the target gene mRNA or protein of a control sample. The desired effect of a miR-specific inhibitor may also be measured by detecting an increase in the expression of genes down-regulated by the miRNA targeted by the miR-specific inhibitor.

By “modulate” is meant that the expression of the gene, or level of RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term “modulate” can mean “inhibit,” but the use of the word “modulate” is not limited to this definition.

As used herein, “RNA” refers to a molecule comprising at least one ribonucleotide residue. The term “ribonucleotide” means a nucleotide with a hydroxyl group at the 2′ position of a β-D-ribofuranose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of an RNAi agent or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

As used herein, the term “complementary” refers to nucleic acid sequences that are capable of base-pairing according to the standard Watson-Crick complementary rules. That is, purines will base pair with pyrimidines to form combinations, e.g. guanine paired with cytosine (G:C) and adenine paired with either thymine (A:T) in the case of DNA, or adenine paired with uracil (A:U) in the case of RNA.

As used herein, the term “essentially complementary” with reference to microRNA target sequences refers to microRNA target nucleic acid sequences that are longer than 8 nucleotides that are complementary (an exact match) to at least 8 consecutive nucleotides of the 5′ portion of a microRNA molecule from nucleotide positions 1 to 10, (also referred to as the “seed region”), and are at least 65% complementary (such as at least 70%, at least 75%, at least 80%, at least 85%, at least 90%, at least 95%, or at least 96% identical) across the remainder of the microRNA target nucleic acid sequence as compared to a naturally occurring miR-106b family member. The comparison of sequences and determination of percent identity and similarity between two sequences can be accomplished using a mathematical algorithm of Karlin and Altschul (1990, PNAS 87:2264-2268), modified as in Karlin and Altschul (1993, PNAS 90:5873-5877). Such an algorithm is incorporated into the NBLAST and XBLAST programs of Altshcul et al. (1990 J. Mol. Biol. 215:403-410).

The term “gene expression”, as used herein, refers to the process of transcription and translation of a gene to produce a gene product, be it RNA or protein. Thus, modulation of gene expression may occur at any one or more of many levels, including transcription, post-transcriptional processing, translation, post-translational modification, and the like.

As used herein, the term “expression cassette” refers to a nucleic acid molecule, which comprises at least one nucleic acid sequence that is to be expressed, along with its transcription and translational control sequences. The expression cassette typically includes restriction sites engineered to be present at the 5′ and 3′ ends such that the cassette can be easily inserted, removed, or replaced in a gene delivery vector. Changing the cassette will cause the gene delivery vector into which it is incorporated to direct the expression of a different sequence.

As used herein, the term “phenotype” encompasses the meaning known to one of skill in the art, including modulation of the expression of one or more genes, as measured by gene expression analysis or protein expression analysis.

As used herein, the term “proliferative disease” or “cancer” as used herein is meant, any disease, condition, trait, genotype or phenotype characterized by unregulated cell growth or replication as is known in the art; including leukemias, for example, acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and any other cancer or proliferative disease, condition, trait, genotype or phenotype that can respond to the modulation of disease related gene expression in a cell or tissue, alone or in combination with other therapies.

As used herein, the term “source of biological knowledge” refers to information that describes the function (e.g., at molecular, cellular and system levels), structure, pathological roles, toxicological implications, etc., of a multiplicity of genes. Various sources of biological knowledge can be used for the methods of the invention, including databases and information collected from public sources such as Locuslink, Unigene, SwissTrEMBL, etc., and organized into a relational database following the concept of the central dogma of molecular biology. In some embodiments, the annotation systems used by the Gene Ontology (G0) Consortium or similar systems are employed. G0 is a dynamic controlled vocabulary for molecular biology which can be applied to all organisms as knowledge of gene function is accumulating and changing, it is developed and maintained by the Gene Ontology™ Consortium (Gene Ontology: tool for the unification of biology. The Gene Ontology Consortium (2000), Nature Genet. 25:25-29)).

As used herein, the term to “inhibit the proliferation of a mammalian cell” means to kill the cell, or permanently or temporarily arrest the growth of the cell. Inhibition of a mammalian cell can be inferred if the number of such cells, either in an in vitro culture vessel, or in a subject, remains constant or decreases after administration of the compositions of the invention. An inhibition of tumor cell proliferation can also be inferred if the absolute number of such cells increases, but the rate of tumor growth decreases.

As used herein, the terms “measuring expression levels,” “obtaining an expression level” and the like, includes methods that quantify a gene expression level of, for example, a transcript of a gene, including microRNA (miRNA) or a protein encoded by a gene, as well as methods that determine whether a gene of interest is expressed at all. Thus, an assay which provides a “yes” or “no” result without necessarily providing quantification, of an amount of expression is an assay that “measures expression” as that term is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see for example, U.S. Pat. No. 5,569,588) nuclease protection, RT-PCR, microarray profiling, Nanostring's NCOUNTER™ Digital Gene Expression System (Seattle, Wash.) (See also WO2007076128; WO2007076129); differential display, 2D gel electrophoresis, SELDI-TOF, ICAT, enzyme assay, antibody assay, and the like.

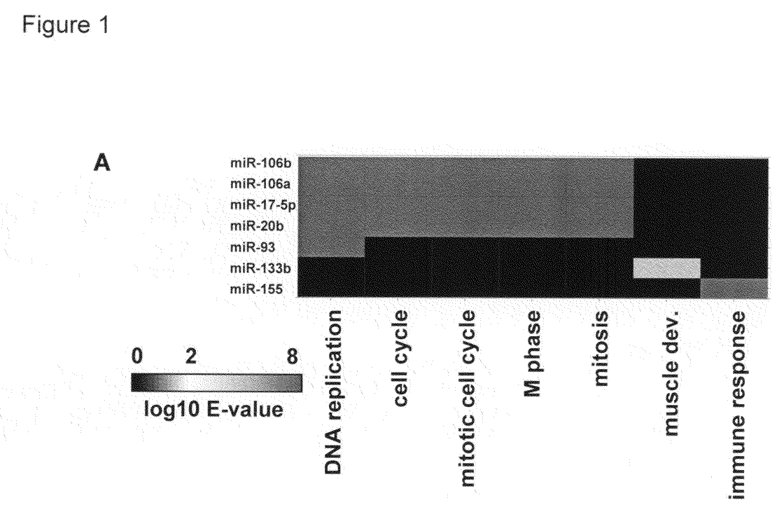

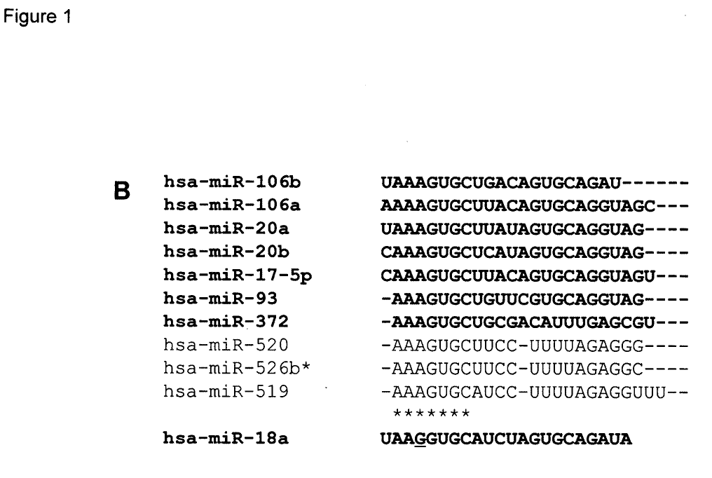

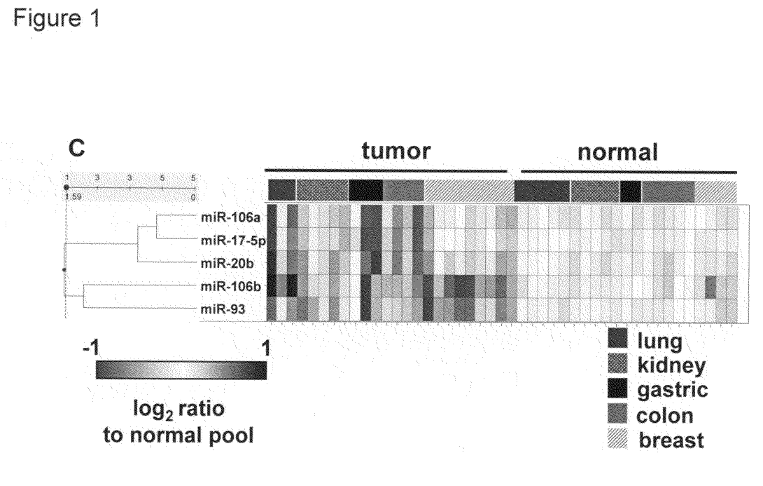

As used herein, “miR-106b family” refers to one or more microRNAs in the miR-106b family, including, but not limited to, miR-106b (SEQ ID NO:1), miR-106a (SEQ ID NO:2), miR-20a (SEQ ID NO:3), miR-20b (SEQ ID NO:4), and miR-17-5p (SEQ ID NO:5). The miR-106b family is the largest family of micro RNAs to date with 18 members (see FIG. 1B) (not shown are 4 miR-520, and 4 miR-519 variants). miR-106b (SEQ ID NO:1), miR-106a (SEQ ID NO:2), miR-20b (SEQ ID NO:4), miR-20a (SEQ ID NO: 3), and miR-17-5p (SEQ ID NO:5) share seed region identity at positions 2-8 of the miRNA and phenotypes (see Examples). miR-93 (SEQ ID NO:18), miR-372 (SEQ ID NO:19), miR-520 (SEQ ID NO:20), miR-526* (SEQ ID NO:21), and miR-519 (SEQ ID NO:22) have seed regions that are off-set by one base at position 1 of the 5′ end, and miR-18 (SEQ ID NO:23) has a seed region with one divergent base at position 4 (see FIG. 1B). These microRNAs do not exhibit the miR-106b phenotypes (see Examples). However, miR-93_{—}2 (SEQ ID NO:7) and miR-372_{—}2 (SEQ ID NO:6), which contain an additional base at the 5′ end such that the “AAAGUGC” miR-106 seed region sequence (SEQ ID NO:8) is shifted in alignment to positions 2-8, show subtle miR-106b phenotypes (see Examples and FIG. 6A). In this application, unless otherwise specified, it will be understood that “miR-106b family” refers to one or more microRNAs in the miR-106b family which share the miR-106b seed region (SEQ ID NO:8) at positions 2-8 and exhibit the cell cycle progression phenotype (acceleration of the G1-to-S transition). In one embodiment, the “miR-106b family” refers to miR-106b (SEQ ID NO:1), miR-106a (SEQ ID NO:2), miR-20a (SEQ ID NO:3), miR-20b (SEQ ID NO:4), and miR-17-5p (SEQ ID NO:5). In another embodiment, the “miR-106b family” refers to miR-106b (SEQ ID NO:1), miR-106a (SEQ ID NO:2), miR-20a (SEQ ID NO:3), miR-20b (SEQ ID NO:4), miR-17-5p (SEQ ID NO:5), miR-93_{—}2 (SEQ ID NO:7), and miR-373_{—}2 (SEQ ID NO:6). In another embodiment, the “miR-106b family” refers to all 18 members.

As used herein, “miR-106” refers to either miR-106a
(SEQ ID NO:2) or miR-106b (SEQ ID NO:1), or both
miRNA species.
As used herein, “miR-106b” refers to “UAAAGUGCUGACA
GUGCAGAU” (SEQ ID NO:1) and precursor RNA sequences
thereof, an example of which is “CCUGCCGGGGCUAAAGUG
CUGACAGUGCAGAUAGUGGUCCUCUCCGUGCUACCGCACUGUGGGUACUUG
CUGCUCCAGCAGG” (SEQ ID NO:24).

As used herein, “miR-106b seed region” refers to the nucleotides at positions 1-10 of the miRNA from the 5′ end. Positions 2-8 of the seed region of the miRNA from the 5′ end comprises the nucleotide sequence “AAAGUGC” (SEQ ID NO: 8). miR-106b (SEQ ID NO:1), miR-106a (SEQ ID NO:2), miR-20 (SEQ ID NOs:3, 4), and miR-17-5p (SEQ ID NO:5) share miR-106b seed region identity at positions 2-8. miR-372_{—}2 (SEQ ID NO:6) and miR-93_{—}2 (SEQ ID NO:7) also share the miR-106b seed region identity at positions 2-8.

As used herein, “miR-106a” refers to “AAAAGUGCUUACA
GUGCAGGUAGC” (SEQ ID NO:2) and precursor RNA
sequences thereof, an example of which is “CCUUGGCC
AUGUAAAAGUGCUUACAGUGCAGGUAGCUUUUUGAGAUCUACUGCAAUGUA
AGCACUUCUUACAUUACCAUGG” (SEQ ID NO:25).
As used herein, “miR-20” refers to miR-20a or
miR-20b, or both miRNA species.
As used herein, “miR-20a” refers to “UAAAGUGCUUAUAG
UGCAGGUAG” (SEQ ID NO:3) and precursor RNA sequen-
ces thereof, an example of which is “GUAGCACUAAAGUG
CUUAUAGUGCAGGUAGUGUUUAGUUAUCUACUGCAUUAUGAGCACUUAAAG
UACUGC” (SEQ ID NO: 26).
As used herein, “miR-20b” refers to “CAAAGUGCUUAUAG
UGCAGGUAG” (SEQ ID NO:4) and precursor RNA sequen-
ces thereof, an example of which is “AGUACCAAAGUGCU
CAUAGUGCAGGUAGUUUUGGCAUGACUCUACUGUAGUAUGGGCACUUCCAG
UACU” (SEQ ID NO: 27).
As used herein “miR-17-5p” refers to “CAAAGUGCUUACA
GUGCAGGUAGU” (SEQ ID NO:5) and precursor RNA se-
quences thereof, an example of which is “GUCAGAAUAA
UGUCAAAGUGCUUACAGUGCAGGUAGUGAUAUGUGCAUCUACUGCAGUGAA
GGCACUUGUAGCAUUAUGGUGAC” (SEQ ID NO: 28).
As used herein, “miR-93” refers to “AAAGUGCUGUUCGUG
CAGGUAG” (SEQ ID NO:18) and precursor RNA sequences
thereof, an example of which is “CUGGGGGCUCCAAAGUGC
UGUUCGUGCAGGUAGUGUGAUUACCCAACCUACUGCUGAGCUAGCACUUCC
CGAGCCCCCGG” (SEQ ID NO: 29).
As used herein, “miR-93_2” refers to “CAAAGUGCUGUUC
GUGCAGGUAG” (SEQ ID NO:7) and precursor sequences
thereof, an example of which is “CUGGGGGCUCCAAAGUGC
UGUUCGUGCAGGUAGUGUGAUUACCCAACCUACUGCUGAGCUAGCACUUCC
CGAGCCCCCGG” (SEQ ID NO: 30).
As used herein, “miR-372” refers to “AAAGUGCUGCGACA
UUUGAGCGU” (SEQ ID NO: 19) and precursor sequences
thereof, an example of which is “GUGGGCCUCAAAUGUGGA
GCACUAUUCUGAUGUCCAAGUGGAAAGUGCUGCGACAUUUGAGCGUCAC"
(SEQ ID NO:31).
As used herein, “miR-372_2” refers to “GAAAGUGCUGCG
ACAUUUGAGCGU” (SEQ ID NO:6) and precursor sequences
thereof, an example of which is “GUGGGCCUCAAAUGUGGA
GCACUAUUCUGAUGUCCAAGUGGAAAGUGCUGCGACAUUUGAGCGUCAC"
(SEQ ID NO:32).

As used herein, “cell cycle progression phenotype”, “miR-106b-family cell cycle phenotype” or “miR-106b phenotype” refers to acceleration of progression through the cell cycle. Acceleration may affect one or more phases in the cell cycle, or one or more transitions from one phase to the next. In most growing eukaryotic cells, the cell cycle comprises four phases, G1, S, G2, and M. In one embodiment, the cell cycle progression phenotype refers to acceleration of the G1-to-S transition. Methods of measuring acceleration or retardation of progression through the cell cycle have been previously described and are known in the art (see Examples).

As used herein, “p21,” also known as CDKN1A, CIP1, WAF1, CAP20, or SDI1, refers to cyclin-dependent kinase inhibitor 1A, which is encoded by NM_{—}078467 (SEQ ID NO:33) or NM_{—}000389 (SEQ ID NO:34). p21 is a transcriptional target for multiple tumor-suppressor signaling cascades, including p53, TGFβ and APC (reviewed in Rowland and Peeper, 2006, Nature Rev. Cancer 6:11-23). p21 prevents cell-cycle progression by inhibiting the activity of cyclin E-associated CDK2. In response to DNA damage signaling, p53 is stabilized and induces expression of many target genes, of which p21 is a crucial target. Up-regulation of p21 leads to G1-arrest following DNA damage.

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequences, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

The terms “over-expression”, “over-expresses”, “over-expressing”, or “gain-of-function” and the like, refer to the state of altering a subject such that expression of one or more genes in said subject is significantly higher, as determined using one or more statistical tests, than the level of expression of said gene or genes in the same unaltered subject or an analogous unaltered subject.

As used herein, a “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, chemically synthesized nucleic acid that has not been purified.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

As used herein, “subject”, as refers to an organism or to a cell sample, tissue sample or organ sample derived therefrom, including, for example, cultured cell lines, biopsy, blood sample, or fluid sample containing a cell. For example, an organism may be an animal, including but not limited to, a cow, a pig, a mouse, a rat, a chicken, a cat, a dog, etc., and is usually a mammal, such as a human.

### II. ASPECTS AND EMBODIMENTS OF THE INVENTION

In accordance with the methods of this invention, the level of at least one miR-106b family member is modulated (i.e. increased or decreased) in a cell type of interest to accelerate or inhibit cell cycle progression. In one embodiment, the level of at least one miR-106b family member is decreased in a cell type of interest. A decrease in miR-106b expression may be achieved using any suitable method, such as introducing a miR-specific inhibitor, such as an iRNA agent selected to inhibit expression of the endogenous gene encoding the miR-106b family member or an anti-miR selected to interact with the miR-106b family member and prevent target transcript binding and cleavage.

In an alternative embodiment, the level of at least one miR-106b family member is modulated (i.e. increased or decreased) in a cell type of interest to up or down regulate one or more genes from Table 2. Alternatively, the level of at least one miR-106b family member is modulated (i.e. increased or decreased) in a cell type of interest to up or down regulate one or more genes from Table 3. In a more specific embodiment, the level of at least one miR-106b family member is modulated to up or down regulate p21, NIKIRAS1, LIMK1, MAPRE3, RNH1, or MAPK1.

In another embodiment, the level of at least one miR-106b family member is increased in a cell type of interest. An increase in expression of a miR-106b family member may be achieved using any suitable method, such as by inducing expression of the endogenous miR-106b family member, by introducing an expression vector encoding a miR-106b family member, or by introducing one or more miR-106b family member synthetic duplex molecules into the cell type of interest.

In one embodiment, the level of at least one miR-106b family member is increased in a cell type of interest by introducing at least one miR-106b family member in the cell. The introduced miR-106b family member may be encoded in an expression vector, or may be a chemically synthesized or recombinantly produced gene product. The miR-106b family member for use in the practice of the methods of the invention can be obtained using a number of standard techniques. For example, the gene products can be chemically synthesized or recombinantly produced as described in more detail below.

The miR-106b family member may be introduced into the cell using various methods such as infection with a viral vector encoding the microRNA, microinjection, or by transfection using electroporation or with the use of a transfection agent. Transfection methods for mammalian cells are well known in the art, and include direct injection of the nucleic acid into the nucleus of a cell, electroporation, liposome transfer or transfer mediated by lipophilic materials, receptor mediated nucleic acid delivery, bioballistic or particle acceleration, calcium phosphosphate precipitation and transfection mediated by viral vectors. For example, cells can be transfected with a liposomal transfer compound, e.g., DOTAP (N-[1-(2,3-dioleoyloxy)propyl]-N,N,N,-trimethyl-ammonium methylsulfate, Boehringer-Mannheim) or an equivalent, such as LIPOFECTIN. An exemplary method for transfecting miRNA into mammalian cells is described in the EXAMPLES.

The methods of this aspect of the invention may be practiced using any cell type, such as primary cells or an established line of cultured cells may be used in the practice of the methods of the invention. For example, the methods may be used in any mammalian cell from a variety of species, such as a cow, horse, mouse, rat, dog, pig, goat, or primate, including a human. In some embodiments, the methods may be used in a mammalian cell type that has been modified, such as a cell type derived from a transgenic animal or a knockout mouse.

In some embodiments, the method of the invention is practiced using a cancer cell type. Representative examples of suitable cancer cell types that can be cultured in vitro and used in the practice of the present invention are colon cancer cells, such as wild type HCT116, wild-type DLD-1, HCT116-Dicer^{ex5} and DLD-1 Dicer^{ex5} cells described in Cummins, J. M., et al., PNAS103(10):3687-3692 (2006)) or breast cancer cells, such as HMEC (human mammary epithelial cells) described in Smith et al., 2007, J. Biol. Chem. 282:2135-43. Other non-limiting examples of suitable cancer cell types include A549, MCF7, and TOV21G and are available from the American Type Culture Collection, Rockville, Md. In further embodiments, the cell type is a microRNA mediated cancer cell type. For example, it has been shown that miR-17, 18, 19, 20, 25, 92, 93 and 106 corresponds to clusters of miRNAs that have been found to be expressed in skeletal muscle and dendritic cells and upregulated by Myc (O'Donnell et al., Nature 435:828 (2005)) and to promote tumor growth in a mouse model of B-cell lymphoma (He et al., Nature 435:828 (2005)).

One embodiment of therapeutic treatment, involves use of a therapeutically sufficient amount of a miR-106b family specific inhibitor to treat tumors classified as containing substantially inactive p21. Such treatment may be in combination with one or more DNA damaging agents. Therapeutic miR-106b family specific inhibitor compositions may comprise a single stranded contiguous nucleotide sequence of at least 18 nucleotides, wherein said sequence comprises a seed region consisting of nucleotide positions 1 to 10, wherein position 1 represents the 5′ end of said guide strand and wherein said seed region comprises a nucleotide sequence of at least six contiguous nucleotides at positions 2-8 that is identical to the miR-106b family seed region (SEQ ID NO: 8).

In order to enhance the stability of the short interfering nucleic acids, the 3′ overhangs can also be stabilized against degradation. In one embodiment, the 3′ overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

Another aspect of the invention provides chemically modified siRNA constructs. For example, the siRNA agent can include a non-nucleotide moiety. A chemical modification or other non-nucleotide moiety can stabilize the sense (passenger strand) and antisense (guide strand) sequences against nucleolytic degradation. Additionally, conjugates can be used to increase uptake and target uptake of the siRNA agent to particular cell types. Thus, in one embodiment the siRNA agent includes a duplex molecule wherein one or more sequences of the duplex molecule is chemically modified. Non-limiting examples of such chemical modifications include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5′-C-methyl nucleotides, and terminal glyceryl and/or inverted deoxy abasic residue incorporation. These chemical modifications, when used in siRNA agents, can help to preserve RNAi activity of the agents in cells and can increase the serum stability of the siRNA agents.

In one embodiment, the first and optionally or preferably the first two internucleotide linkages at the 5′ end of the antisense and/or sense sequences are modified, preferably by a phosphorothioate. In another embodiment, the first, and perhaps the first two, three, or four internucleotide linkages at the 3′ end of a sense and/or antisense sequence are modified, for example, by a phosphorothioate. In another embodiment, the 5′ end of both the sense and antisense sequences, and the 3′ end of both the sense and antisense sequences are modified as described.

Another aspect of the invention provides a method of inhibiting proliferation of a mammalian cell comprising introducing into said cell an effective amount of a miR-specific inhibitor of at least one miR-106b family member, wherein said miR-specific inhibitor comprises nucleotide sequence of at least 18 nucleotides, wherein said nucleotide sequence comprises at least 6 consecutive nucleotides that are complementary to positions 2-8 of a seed region of said miR-106b family member (SEQ ID NO:8), and has at least 50%, 60%, 70%, 80% or 90% complementarity to the rest of said miR-106b family member sequence, and wherein said miR-specific inhibitor retards the G1-to-S transition.

In one embodiment, the miR-specific inhibitor is an anti-miR molecule that is introduced into said cell by transfection. In some embodiments, the introduced miR-specific inhibitors includes one or more chemically modified nucleotides. An effective amount of miR-specific inhibitors, is the amount sufficient to cause a measurable change in the detected level of one or more microRNAs that are targeted by the miR-specific inhibitor, in one or more gene transcripts regulated by one or more members of the miR-106b family (i.e. reverses target gene regulation observed by the miRNA), or reverses the miR-106b family phenotype on cell cycle progression (accelerating G1-to-S transition). In one embodiment, the gene transcripts regulated by one or more members of the miR-106b family or miR-106b family specific inhibitor are selected from Table 2. In another embodiment, the gene transcripts regulated by the one or more members of the miR-106b family or miR-106b family specific inhibitor are selected from Table 3. Examples of anti-miRs useful for inhibiting miRNAs are well known in the art (WO2005054494; Hutvagner et al., 2004, PLoS Biol. 2:e98; Meister et al., 2004, RNA 10:544-50; Orom et al., 2006, Gene 372:137-41; WO2005079397; Krutzfeldt et al., 2005, Nature 438:685-689; Davis et al, 2006; Nucleic Acid Res. 34:2294-2304;Esau et al., 2006, Cell Metab. 3:87-98).

In another embodiment, cell division is inhibited by introduction of a target mimics, comprising at least one nucleotide sequence comprising 6 consecutive nucleotides that are complementary to positions 2-8 of the miR-106 family seed region (SEQ ID NO:8). Target mimics may contain multiple nucleotide sequences comprising 6 consecutive nucleotides that are complementary to positions 2-8 of the miR-106 family seed region (SEQ ID NO:8). The target mimic may be vector encoded. The target mimic may also comprise mismatches at positions 9-12 to prevent miRNA cleavage of the target mimic. Examples of target mimics useful for inhibiting miRNAs are known in the art (Franco-Zorrilla et al, 2007, Nature Genet. 39:1033-1037; Ebert et al., 2007, Nature Methods 4:721-6). An effective amount of a target mimic, is the amount sufficient to cause a measurable change in the detected level of one or more microRNAs that are targeted by the target mimic, one or more gene transcripts regulated by one or more members of the miR-106b family, or reverses the miR-106b family phenotype on cell cycle progression.

In one embodiment, the gene transcripts regulated by one or more members of the miR-106b family or miR-106b family specific inhibitor are selected from Table 2. In another embodiment, the gene transcripts regulated by the one or more members of the miR-106b family or miR-106b family specific inhibitor are selected from Table 3.

Another aspect of the invention, provides a method of treating a disease associated with a p21 defect of a mammalian cell, for example, cancer, comprising introducing into said cell an effective amount of a miR-specific inhibitor of at least one miR-106b family member (to up-regulate p21) or an siNA (to down-regulate p21), wherein the siNA comprises a guide strand contiguous nucleotide sequence of at least 18 nucleotides, wherein said guide strand comprises a seed region consisting of nucleotide positions 1 to 10, wherein position 1 represents the 5′ end of said guide strand and wherein said seed region comprises a nucleotide sequence of at least six contiguous nucleotides at positions 2-8 that is identical to SEQ ID NO:8, wherein said miR-specific inhibitor or siNA retards or accelerates the G1-to-S transition, respectively. An effective amount of miR-specific inhibitors or siNA, is the amount sufficient to cause a measurable change in the detected level of one or more microRNAs that are targeted by the miR-specific inhibitor, in one or more gene transcripts regulated by one or more members of the miR-106b family, or modulates the G1-to-S transition. In one embodiment, the gene transcripts regulated by one or more members of the miR-106b family or miR-106b family specific inhibitor are selected from Table 2. In another embodiment, the gene transcripts regulated by the one or more members of the miR-106b family or miR-106b family specific inhibitor are selected from Table 3.

In one embodiment, the siNA is a duplex RNA molecule that is introduced into said cell by transfection. In some embodiments, the introduced siNA includes one or more chemically modified nucleotides. In one embodiment, the miR-specific inhibitor is an anti-miR that is introduced into said cell by transfection. In some embodiments, the introduced anti-miR includes one or more chemically modified nucleotides. An effective amount of siNA, is the amount sufficient to cause a measurable change in the detected level of one or more gene transcripts that are regulated by one or more members of the miR-106b family or accelerate the G1-to-S transition. In one embodiment, the gene transcripts regulated by one or more members of the miR-106b family are selected from Table 2. In another embodiment, the gene transcripts are selected from Table 3.

In another embodiment, the disease associated with a p21 defect is treated by introduction of a nucleic acid vector molecule expressing a shRNA gene, wherein the shRNA transcription product acts as an RNAi agent. The shRNA gene may encode a microRNA precursor RNA, such as, for example, SEQ ID NO:24. Alternatively, the shRNA gene may encode any other RNA sequence that is susceptible to processing by endogenous cellular RNA processing enzymes into an active siRNA sequence, wherein the seed region of the active siRNA sequence contains at least a six contiguous nucleotide sequence at positions 2-8 of the guide strand that is identical SEQ ID NO:8. An effective amount of shRNA, is the amount sufficient to cause a measurable change in the detected level of one or more gene transcripts that are regulated by one or more members of the miR-106b family. In one embodiment, the gene transcripts regulated by one or more members of the miR-106b family are selected from Table 2. In another embodiment, the gene transcripts are selected from Table 3.

Another aspect of the invention provides a method of at least partially restoring p21 function of a mammalian cell comprising introducing into said cell an effective amount of a miR-specific inhibitor of at least one miR-106b family member, wherein said miR-specific inhibitor comprises nucleotide sequence of at least 18 nucleotides, wherein said nucleotide sequence comprises at least 6 consecutive nucleotides that are complementary to positions 2-8 of a seed region of said miR-106b family member (SEQ ID NO:8), and has at least 50%, 60%, 70%, 80% or 90% complementarity to the rest of said miR-106b family member sequence, and wherein said miR-specific inhibitor retards the G1-to-S transition.

In one embodiment, the miR-specific inhibitor is an anti-miR molecule that is introduced into said cell by transfection. In some embodiments, the introduced miR-specific inhibitor includes one or more chemically modified nucleotides. An effective amount of miR-specific inhibitor is the amount sufficient to cause a measurable change in the detected level of one or more microRNAs that are targeted by the miR-specific inhibitor, in the detected level of one or more gene transcripts that are regulated by one or more members of the miR-106b family, or retards the G-to-S transition. In one embodiment, the gene transcripts regulated by one or more members of the miR-106b family are selected from Table 2. In another embodiment, the gene transcripts are selected from Table 3. In another embodiment, the p21 defect is at least partially restored by introduction of target mimics.

In another aspect, the invention provides an isolated nucleic acid molecule comprising, or consisting essentially of, a guide strand nucleotide sequence of 18 to 25 nucleotides, said guide strand nucleotide sequence comprising a seed region nucleotide sequence and a non-seed region nucleotide sequence, said seed region consisting essentially of nucleotide positions 1 to 10 and said non-seed region consisting essential of nucleotide positions 11 to the 3′ end of said guide strand, wherein position 1 of said guide strand represents the 5′ end of said guide strand, wherein said seed region further comprises a consecutive nucleotide sequence of at least 6 nucleotides at positions 2-8 that is identical to a nucleotide SEQ ID NO:8, and wherein said isolated nucleic acid molecule accelerates the G1-to-S transition. In one embodiment, said guide strand nucleotide sequence outside of positions 2-8 has at least 50%, 60%, 70%, 80%, 90%, or 100% identity to the rest of a miR-106b family member. The guide strand may or may not have the same number of nucleotides as the miR-106b family member. The isolated nucleic acid molecule may be single stranded or double stranded. These isolated nucleic acid molecules may be used as synthetic mimics of miR-106b family members, to accelerate G1-to-S transition, to down-regulate p21, or to down regulate the gene transcripts listed in Table 2 or Table 3.

In another embodiment, the isolated nucleic acid molecule consists essentially of a guide strand nucleotide sequence of 19 to 23 nucleotides, said guide strand nucleotide sequence comprising a seed region nucleotide sequence and a non-seed region nucleotide sequence, said seed region consisting essentially of nucleotide positions 1 to 10 and said non-seed region consisting essential of nucleotide positions 11 to the 3′ end of said guide strand, wherein position 1 of said guide strand represents the 5′ end of said guide strand, wherein said seed region further comprises a consecutive nucleotide sequence of at least 6 nucleotides at positions 2-8 that is identical in sequence to SEQ ID NO: 8, and wherein said isolated nucleic acid molecule accelerates the G1-to-S transition.

Another aspect of the present invention provides a method for determining the proliferative status of a cell sample isolated from a subject. miR-106b family members may be used as biomarkers for cell cycle progression phenotype. High miR-106b expression correlates with important characteristics of tumors, i.e. acceleration of cell cycle progression (i.e. G1-to-S phase). Accelerated cell cycle progression is a hallmark of highly proliferative cells. In one embodiment, the method comprises obtaining a cell sample from a subject, measuring the expression levels of miR-106b family member in the cell sample, and comparing the measured expression levels of the miR-106b family member to a proliferation reference value, wherein expression levels of miR-106b above the proliferation reference value are indicative of accelerated cell cycle progression. In one embodiment, the proliferation reference value may be derived from adjacent normal tissue from said subject. In another embodiment, the proliferation reference value may be derived from a pool of normal samples from one or more subjects. In another embodiment, the miR-106b family member may comprise one or more of the following: miR-106b, miR-106a, miR-20a, miR-20b, miR-17-5p, miR-93_{—}2, and miR-372_{—}2.

In one embodiment, the expression level of a miR-106b family member is determined by measuring the amount of the mature microRNA. The amount of miR-106b family member present in cells or tissues can be measured using methods such as nucleic acid hybridization (Lu et al., 2005, Nature 435:834-838); quantitative PCR (Raymond et al., 2006, RNA 11:1737-1744), incorporated by reference, or any other method that is capable of providing a measured level (either as a quantitative amount or as an amount relative to a standard or control amount, i.e., a ratio or a fold-change) of a miRNA within a cell or sample. In another embodiment, the expression level of a miR-106b family member is determined by the amount of the primary transcript, pri-mi-106b family member. The amount of pri-mi-106b family member present in cells or samples can be measured using methods such as gene expression profiling using microarrays (Jakcson et al., 2003, Nat. Biotech. 21:635-637) or any other method that is capable of providing a measured level (either as a quantitative amount or as an amount relative to a standard or control amount, i.e., a ratio or a fold-change) of an RNA within a cell or sample. In another embodiment, the expression level of a miR-106b family member is determined by measuring the amount of the stem-loop precursor, pre-miR-106b.

Differences between the measured level of miR-106b family member in the cell sample and the proliferative reference value is evaluated using one or more statistical tests known in the art. Methods of comparison to reference values have been previously described in PCT Application “Compositions Comprising miR34Therapeutic Agents for Treating Cancer,” by Michele Cleary et al., filed on May 5, 2008, incorporated herein by reference; and Provisional Application “Methods of Using miR-210 as a Biomarker for Hypoxia and as a Therapeutic Agent for Treating Cancer,” by Zhan Zhang et al., filed on Apr. 24, 2008, incorporated here by reference.

### III. NUCLEIC ACID MOLECULES

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992, “DNA replication,” Freeman and Company, New York, N.Y.).

As used herein, a “nucleotide” refers to a nucleoside further comprising a “backbone moiety.” A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. Other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, “Nucleotide Analogs: Synthesis and Biological Function,” Wiley, N.Y.).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake and regulating RNA expression; U.S. Pat. No. 5,858,988, which describes hydrophobic carrier agent attached to the 2′-O position of oligonucleotides to enhance their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′deoxyguanosine nucleotides and nucleic acid analogs thereof).

shRNA Mediated Suppression

Alternatively, certain of the nucleic acid molecules of the instant invention can be expressed within cells from eukaryotic promoters (e.g., Izant and Weintraub, 1985, Science, 229: 345; McGarry and Lindquist, 1986, Proc. Natl. Acad. Sci., USA 83:399; Scanlon et al., 1991, Proc. Natl. Acad. Sci. USA, 88:10591-95; Kashani-Sabet et al., 1992, Antisense Res. Dev., 2:3-15; Dropulic et al., 1992, J. Virol., 66:1432-41; Weerasinghe et al., 1991, J. Virol., 65:5531-4; Ojwang et al., 1992, Proc. Natl. Acad. Sci. USA, 89:10802-06; Chen et al., 1992, Nucleic Acids Res., 20:4581 89; Sarver et al., 1990 Science, 247:1222-25; Thompson et al, 1995, Nucleic Acids Res., 23:2259; Good et al., 1997, Gene Therapy, 4:45). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27:15-6; Taira et al., 1991, Nucleic Acids Res., 19:5125-30; Ventura et al., 1993, Nucleic Acids Res., 21:3249-55; Chowrira et al., 1994, J. Biol. Chem., 269:25856). Gene therapy approaches specific to the CNS are described by Blesch et al., 2000, Drug News Perspect., 13:269-280; Peterson et al., 2000, Cent. Nerv. Syst. Dis., 485:508; Peel and Klein, 2000, J. Neurosci. Methods, 98:95-104; Hagihara et al., 2000, Gene Ther., 7:759-763; and Herrlinger et al., 2000, Methods Mol. Med., 35:287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient or subject followed by reintroduction into the patient or subject, or by any other means that would allow for introduction into the desired target cell (for a review see Couture et al., 1996, TIG., 12:510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operably linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid molecules of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid molecule of the invention; and/or an intron (intervening sequences).

Transcription of the nucleic acid molecule sequences are driven from a promoter for eukaryotic RNA polymerase I (pol 1), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87:6743-7; Gao and Huang 1993, Nucleic Acids Res., 21:2867-72; Lieber et al., 1993, Methods Enzymol., 217:47-66; Zhou et al., 1990, Mol. Cell. Biol., 10:4529-37).

Several investigators have demonstrated that nucleic acid molecules encoding shRNAs or microRNAs expressed from such promoters can function in mammalian cells (Brummelkamp et al., 2002 Science 296:550-553; Paddison et al., 2004, Nat. Methods 1:163-67; McIntyre and Fanning 2006 BMC Biotechnology (Jan 5) δ: 1; Taxman et al., 2006 BMC Biotechnology (Jan 24) 6:7). The above shRNA or microRNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule.

In yet another embodiment the expression vector comprises: a) a transcription initiation region;

b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### IV. MODIFIED siNA MOLECULES

Any of the siNA constructs described herein can be evaluated and modified as described below.

An siNA construct may be susceptible to cleavage by an endonuclease or exonuclease, such as, for example, when the siNA construct is introduced into the body of a subject. Methods can be used to determine sites of cleavage, e.g., endo- and exonucleolytic cleavage on an RNAi construct and to determine the mechanism of cleavage. A siNA construct can be modified to inhibit such cleavage.

Exemplary modifications include modifications that inhibit endonucleolytic degradation, including the modifications described herein. Particularly favored modifications include: 2′ modification, e.g., a 2′-O-methylated nucleotide or 2′-deoxy nucleotide (e.g., 2′deoxy-cytodine), or a 2′-fluoro, difluorotoluoyl, 5-Me-2′-pyrimidines, 5-allyamino-pyrimidines, 2′-O-methoxyethyl, 2′-hydroxy, or 2′-ara-fluoro nucleotide, or a locked nucleic acid (LNA), extended nucleic acid (ENA), hexose nucleic acid (HNA), or cyclohexene nucleic acid (CeNA). In one embodiment, the 2′ modification is on the uridine of at least one 5′-uridine-adenine-3′ (5′-UA-3′) dinucleotide, at least one 5′-uridine-guanine-3′ (5′-UG-3′) dinucleotide, at least one 5′-uridine-uridine-3′ (5′-UU-3′) dinucleotide, or at least one 5′-uridine-cytidine-3′ (5′-UC-3′) dinucleotide, or on the cytidine of at least one 5′-cytidine-adenine-3′ (5′-CA-3′) dinucleotide, at least one 5′-cytidine-cytidine-3′ (5′-CC-3′) dinucleotide, or at least one 5′-cytidine-uridine-3′ (5′-CU-3′) dinucleotide. The 2′ modification can also be applied to all the pyrimidines in an siNA construct. In one preferred embodiment, the 2′ modification is a 2′OMe modification on the sense strand of an siNA construct. In a more preferred embodiment the 2′ modification is a 2′ fluoro modification, and the 2′ fluoro is on the sense (passenger) or antisense (guide) strand or on both strands.

Modification of the backbone, e.g., with the replacement of an O with an S, in the phosphate backbone, e.g., the provision of a phosphorothioate modification can be used to inhibit endonuclease activity. In some embodiments, an siNA construct has been modified by replacing one or more ribonucleotides with deoxyribonucleotides. Preferably, adjacent deoxyribonucleotides are joined by phosphorothioate linkages, and the siNA construct does not include more than four consecutive deoxyribonucleotides on the sense or the antisense strands. Replacement of the U with a C5 amino linker; replacement of an A with a G (sequence changes are preferred to be located on the sense strand and not the antisense strand); or modification of the sugar at the 2′, 6′, 7′, or 8′ position can also inhibit endonuclease cleavage of the siNA construct. Preferred embodiments are those in which one or more of these modifications are present on the sense but not the antisense strand, or embodiments where the antisense strand has fewer of such modifications.

Exemplary modifications also include those that inhibit degradation by exonucleases. In one embodiment, an siNA construct includes a phosphorothioate linkage or P-alkyl modification in the linkages between one or more of the terminal nucleotides of an siNA construct. In another embodiment, one or more terminal nucleotides of a siNA construct include a sugar modification, e.g., a 2′ or 3′ sugar modification. Exemplary sugar modifications include, for example, a 2′-O-methylated nucleotide, 2′-deoxy nucleotide (e.g., deoxy-cytodine), 2′-deoxy-2′-fluoro (2′-F) nucleotide, 2′-O-methoxyethyl (2′-O-MOE), 2′-O-aminopropyl (2′-O-AP), 2′-O—N-methylacetamido (2′-O-NMA), 2′-O-dimethylaminoethlyoxyethyl (2′-DMAEOE), 2′-O-dimethylaminoethyl (2′-O-DMAOE), 2′-O-dimethylaminopropyl (2′-O-AP), 2′-hydroxy nucleotide, or a 2′-ara-fluoro nucleotide, or a locked nucleic acid (LNA), extended nucleic acid (ENA), hexose nucleic acid (HNA), or cyclohexene nucleic acid (CeNA). A 2′ modification is preferably 2′OMe, more preferably, 2′fluoro.

The modifications described to inhibit exonucleolytic cleavage can be combined onto a single siNA construct. For example, in one embodiment, at least one terminal nucleotide of an siNA construct has a phosphorothioate linkage and a 2′ sugar modification, e.g., a 2° F. or 2′OMe modification. In another embodiment, at least one terminal nucleotide of an siNA construct has a 5′ Me-pyrimidine and a 2′ sugar modification, e.g., a 2° F. or 2′OMe modification.

To inhibit exonuclease cleavage, an siNA construct can include a nucleobase modification, such as a cationic modification, such as a 3′-abasic cationic modification. The cationic modification can be, e.g., an alkylamino-dT (e.g., a C6 amino-dT), an allylamino conjugate, a pyrrolidine conjugate, a pthalamido or a hydroxyprolinol conjugate, on one or more of the terminal nucleotides of the siNA construct. In one embodiment, an alkylamino-dT conjugate is attached to the 3′ end of the sense or antisense strand of an RNAi construct. In another embodiment, a pyrrolidine linker is attached to the 3′ or 5′ end of the sense strand, or the 3′ end of the antisense strand. In one embodiment, an allyl amine uridine is on the 3′ or 5′ end of the sense strand, and not on the 5′ end of the antisense strand.

In one embodiment, the siNA construct includes a conjugate on one or more of the terminal nucleotides of the siNA construct. The conjugate can be, for example, a lipophile, a terpene, a protein binding agent, a vitamin, a carbohydrate, a retinoid, or a peptide. For example, the conjugate can be naproxen, nitroindole (or another conjugate that contributes to stacking interactions), folate, ibuprofen, cholesterol, retinoids, PEG, or a C5 pyrimidine linker. In other embodiments, the conjugates are glyceride lipid conjugates (e.g. a dialkyl glyceride derivatives), vitamin E conjugates, or thio-cholesterols. In one embodiment, conjugates are on the 3′ end of the antisense strand, or on the 5′ or 3′ end of the sense strand and the conjugates are not on the 3′ end of the antisense strand and on the 3′ end of the sense strand.

In one embodiment, the conjugate is naproxen, and the conjugate is on the 5′ or 3′ end of the sense or antisense strands. In one embodiment, the conjugate is cholesterol, and the conjugate is on the 5′ or 3′ end of the sense strand and not present on the antisense strand. In some embodiments, the cholesterol is conjugated to the siNA construct by a pyrrolidine linker, or serinol linker, aminooxy, or hydroxyprolinol linker. In other embodiments, the conjugate is a dU-cholesterol, or cholesterol is conjugated to the siNA construct by a disulfide linkage. In another embodiment, the conjugate is cholanic acid, and the cholanic acid is attached to the 5′ or 3′ end of the sense strand, or the 3′ end of the antisense strand. In one embodiment, the cholanic acid is attached to the 3′ end of the sense strand and the 3′ end of the antisense strand. In another embodiment, the conjugate is PEG5, PEG20, naproxen or retinal.

In another embodiment, one or more terminal nucleotides have a 2′-5′ linkage. In certain embodiments, a 2′-5′ linkage occurs on the sense strand, e.g., the 5′ end of the sense strand.

In one embodiment, a siNA construct includes an L-sugar, preferably at the 5′ or 3′ end of the sense strand.

In one embodiment, a siNA construct includes a methylphosphonate at one or more terminal nucleotides to enhance exonuclease resistance, e.g., at the 3′ end of the sense or antisense strands of the construct.

In one embodiment, an siRNA construct has been modified by replacing one or more ribonucleotides with deoxyribonucleotides. In another embodiment, adjacent deoxyribonucleotides are joined by phosphorothioate linkages. In one embodiment, the siNA construct does not include more than four consecutive deoxyribonucleotides on the sense or the antisense strands. In another embodiment, all of the ribonucleotides have been replaced with modified nucleotides that are not ribonucleotides.

In some embodiments, an siNA construct having increased stability in cells and biological samples includes a difluorotoluoyl (DFT) modification, e.g., 2,4-difluorotoluoyl uracil, or a guanidine to inosine substitution.

The methods can be used to evaluate a candidate siNA, e.g., a candidate siRNA construct, which is unmodified or which includes a modification, e.g., a modification that inhibits degradation, targets the dsRNA molecule, or modulates hybridization. Such modifications are described herein. A cleavage assay can be combined with an assay to determine the ability of a modified or non-modified candidate to silence the target transcript. For example, one might (optionally) test a candidate to evaluate its ability to silence a target (or off-target sequence), evaluate its susceptibility to cleavage, modify it (e.g., as described herein, e.g., to inhibit degradation) to produce a modified candidate, and test the modified candidate for one or both of the ability to silence and the ability to resist degradation. The procedure can be repeated. Modifications can be introduced one at a time or in groups. It will often be convenient to use a cell-based method to monitor the ability to silence a target RNA. This can be followed by a different method, e.g., a whole animal method, to confirm activity.

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344:565; Pieken et al., 1991, Science 253:314; Usman and Cedergren, 1992, Trends in Biochem. Sci. 17:334; Burgin et al., 1996, Biochemistry, 35:14090; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Vargeese et al., US 2006/021733). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

Chemically modified siNA molecules for use in modulating or attenuating expression of one or more genes regulated by one or more miR-106b family member are also within the scope of the invention. Described herein are isolated siNA agents, e.g., RNA molecules (chemically modified or not, double-stranded, or single-stranded) that mediate RNAi to inhibit expression of one or more genes that are regulated by one or more miR-106b family members.

The siNA agents discussed herein include otherwise unmodified RNA as well as RNAs which have been chemically modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al., 1994, Nucleic Acids Res. 22:2183-2196. Such rare or unusual RNAs, often termed modified RNAs (apparently because they are typically the result of a post-transcriptional modification) are within the term unmodified RNA, as used herein.

Modified RNA as used herein refers to a molecule in which one or more of the components of the nucleic acid, namely sugars, bases, and phosphate moieties that are the components of the RNAi duplex, are different from that which occur in nature, preferably different from that which occurs in the human body. While they are referred to as modified “RNAs,” they will of course, because of the modification, include molecules which are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of all of the above are discussed herein.

Modifications described herein can be incorporated into any double-stranded RNA and RNA-like molecule described herein, e.g., an siNA construct. It may be desirable to modify one or both of the antisense and sense strands of an siNA construct. As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not.

By way of example, a modification may occur at a 3′ or 5′ terminal position, may occur in a terminal region, e.g. at a position on a terminal nucleotide or in the last 2, 3, 4, 5, or 10 nucleotides of a strand. A modification may occur in a double strand region, a single strand region, or in both. For example, a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, a modification may occur on an internal residue to the exclusion of adjacent residues. In some cases, the sense and antisense strand will have the same modifications or the same class of modifications, but in other cases the sense and antisense strand will have different modifications, e.g., in some cases it may be desirable to modify only one strand, e.g. the sense strand. In some cases, the sense strand may be modified, e.g., capped in order to promote insertion of the anti-sense strand into the RISC complex.

Other suitable modifications that can be made to a sugar, base, or backbone of a siNA construct are described in US2006/0217331, US2005/0020521, WO2003/70918, WO2005/019453, PCT Application No. PCT/US2004/01193. A siNA construct can include a non-naturally occurring base, such as the bases described in any one of the above mentioned references. See also PCT Application No. PCT/US2004/011822. A siNA construct can also include a non-naturally occurring sugar, such as a non-carbohydrate cyclic carrier molecule. Exemplary features of non-naturally occurring sugars for use in siNA agents are described in PCT Application No. PCT/US2004/11829.

Two prime objectives for the introduction of modifications into siNA constructs of the invention is their stabilization towards degradation in biological environments and the improvement of pharmacological properties, e.g. pharmacodynamic properties. There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17:34; Usman et al., 1994, Nucleic Acids Symp. Ser. 31:163; Burgin et al., 1996, Biochemistry, 35:14090). Sugar modification of nucleic acid molecules have been extensively described in the art (see Eckstein et al., International Publication PCT No. WO 92/07065; Perrault et al. 1990, Nature, 344:565-568; Pieken et al., 1991, Science 253:314-317; Usman and Cedergren, 1992, Trends in Biochem. Sci. 17, 334-339; Usman et al. International Publication PCT No. WO 93/15187; Sproat, U.S. Pat. No. 5,334,711 and Beigelman et al., 1995, J. Biol. Chem., 270:25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39:1131; Earnshaw and Gait, 1998, Biopolymers (Nucleic Acid Sciences), 48:39-55; Verma and Eckstein, 1998, Annu. Rev. Biochem., 67:99-134; and Burlina et al., 1997, Bioorg. Med. Chem., 5:1999-2010). Such publications describe general methods and strategies to determine the location of incorporation of sugar, base and/or phosphate modifications and the like into nucleic acid molecules without modulating catalysis. In view of such teachings, similar modifications can be used as described herein to modify the siNA molecules of the instant invention so long as the ability of siNA to promote RNAi in cells is not significantly inhibited.

Modifications may be modifications of the sugar-phosphate backbone. Modifications may also be modification of the nucleoside portion. Optionally, the sense strand is a RNA or RNA strand comprising 5%, 10%, 20%, 30%, 40%, 50%, 60%, 70%, 80%, 90% or 100% modified nucleotides. In one embodiment, the sense polynucleotide is an RNA strand comprising a plurality of modified ribonucleotides. Likewise, in other embodiments, the RNA antisense strand comprises one or more modifications. For example, the RNA antisense strand may comprise no more than 5%, 10%, 20%, 30%, 40%, 50% or 75% modified nucleotides. The one or more modifications may be selected so as increase the hydrophobicity of the double-stranded nucleic acid, in physiological conditions, relative to an unmodified double-stranded nucleic acid having the same designated sequence.

In certain embodiments, the siNA construct comprising the one or more modifications has a logP value at least 0.5 logP units less than the logP value of an otherwise identical unmodified siRNA construct. In another embodiment, the siNA construct comprising the one or more modifications has at least 1, 2, 3 or even 4 logP units less than the logP value of an otherwise identical unmodified siRNA construct. The one or more modifications may be selected so as increase the positive charge (or increase the negative charge) of the double-stranded nucleic acid, in physiological conditions, relative to an unmodified double-stranded nucleic acid having the same designated sequence. In certain embodiments, the siNA construct comprising the one or more modifications has an isoelectric pH (pI) that is at least 0.25 units higher than the otherwise identical unmodified siRNA construct. In another embodiment, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety and a 2′-deoxy-2′fluoride moiety.

In certain embodiments, the RNAi construct is a hairpin nucleic acid that is processed to an siRNA inside a cell. Optionally, each strand of the double-stranded nucleic acid may be 19-100 base pairs long, and preferably 19-50 or 19-30 base pairs long.

An siNAi construct can include an internucleotide linkage (e.g., the chiral phosphorothioate linkage) useful for increasing nuclease resistance. In addition, or in the alternative, an siNA construct can include a ribose mimic for increased nuclease resistance. Exemplary internucleotide linkages and ribose mimics for increased nuclease resistance are described in PCT Application No. PCT/US2004/07070.

An siRNAi construct can also include ligand-conjugated monomer subunits and monomers for oligonucleotide synthesis. Exemplary monomers are described, for example, in U.S. application Ser. No. 10/916,185.

An siNA construct can have a ZXY structure, such as is described in co-owned PCT Application No. PCT/US2004/07070. Likewise, an siNA construct can be complexed with an amphipathic moiety. Exemplary amphipathic moieties for use with siNA agents are described in PCT Application No. PCT/US2004/07070.

The sense and antisense sequences of an siNAi construct can be palindromic. Exemplary features of palindromic siNA agents are described in PCT Application No. PCT/US2004/07070.

In another embodiment, the siNA constructs of the invention can be complexed to a delivery agent that features a modular complex. The complexes can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane); and (c) a targeting group, e.g., a cell or tissue targeting agent, e.g., a lectin, glycoprotein, lipid or protein, e.g., an antibody, that binds to a specified cell type. iRNA agents complexed to a delivery agent are described in PCT Application No. PCT/US2004/07070.

The siNA construct of the invention can have non-canonical pairings, such as between the sense and antisense sequences of the iRNA duplex. Exemplary features of non-canonical iRNA agents are described in PCT Application No. PCT/US2004/07070.

In one embodiment, nucleic acid molecules of the invention include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex, see for example, Lin and Matteucci, 1998, J. Am. Chem. Soc., 120:8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands.

In another embodiment, nucleic acid molecules of the invention include one or more (e.g., about 1, 2, 3, 4, 5, 6, 7, 8, 9, 10, or more) LNA “locked nucleic acid” nucleotides such as a 2′,4′-C methylene bicyclo nucleotide (see for example Wengel et al., International PCT Publication No. WO 00/66604 and WO 99/14226).

An siNA agent of the invention, can be modified to exhibit enhanced resistance to nucleases. An exemplary method proposes identifying cleavage sites and modifying such sites to inhibit cleavage. Exemplary dinucleotides 5′-UA-3′,5′-UG-3′,5′-CA-3′,5′-UU-3′, or 5′-CC-3′ as disclosed in PCT/US2005/018931 may serve as a cleavage site.

For increased nuclease resistance and/or binding affinity to the target, an siRNA agent, e.g., the sense and/or antisense strands of the iRNA agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R.dbd.H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e., deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. In one embodiment, the substitutents are 2′-methoxyethyl, 2′-OCH₃,2′-O-allyl, 2′-C-allyl, and 2′-fluoro.

In another embodiment, to maximize nuclease resistance, the 2′ modifications may be used in combination with one or more phosphate linker modifications (e.g., phosphorothioate). The so-called “chimeric” oligonucleotides are those that contain two or more different modifications.

In certain embodiments, all the pyrimidines of an siNA agent carry a 2′-modification, and the molecule therefore has enhanced resistance to endonucleases. Enhanced nuclease resistance can also be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. The siNA agent can include at least 2, at least 3, at least 4 or at least 5 of such dinucleotides. In some embodiments, the 5′-most pyrimidines in all occurrences of the sequence motifs 5′-UA-3′,5′-CA-3′,5′-UU-3′, and 5′-UG-3′ are 2′-modified nucleotides. In other embodiments, all pyrimidines in the sense strand are 2′-modified nucleotides, and the 5′-most pyrimidines in all occurrences of the sequence motifs 5′-UA-3′ and 5′-CA-3′. In one embodiment, all pyrimidines in the sense strand are 2′-modified nucleotides, and the 5′-most pyrimidines in all occurrences of the sequence motifs 5′-UA-3′,5′-CA-3′, 5′-UU-3′, and 5′-UG-3′ are 2′-modified nucleotides in the antisense strand. The latter patterns of modifications have been shown to maximize the contribution of the nucleotide modifications to the stabilization of the overall molecule towards nuclease degradation, while minimizing the overall number of modifications required to a desired stability, see PCT/US2005/018931. Additional modifications to enhance resistance to nucleases may be found in US2005/0020521, WO2003/70918, WO2005/019453.

The inclusion of furanose sugars in the oligonucleotide backbone can also decrease endonucleolytic cleavage. Thus, in one embodiment, the siNA of the invention can be modified by including a 3′ cationic group, or by inverting the nucleoside at the 3′-terminus with a 3′-3′ linkage. In another alternative, the 3′-terminus can be blocked with an aminoalkyl group, e.g., a 3′C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

Similarly, 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′-end of oligonucleotide. Even small alkyl chains, aryl groups, or heterocyclic conjugates or modified sugars (D-ribose, deoxyribose, glucose etc.) can block 3′-5′-exonucleases.

An alternative approach to increasing resistance to a nuclease by an siNA molecule proposes including an overhang to at least one or both strands of an duplex siNA. In some embodiments, the nucleotide overhang includes 1 to 4, preferably 2 to 3, unpaired nucleotides. In another embodiment, the unpaired nucleotide of the single-stranded overhang that is directly adjacent to the terminal nucleotide pair contains a purine base, and the terminal nucleotide pair is a G-C pair, or at least two of the last four complementary nucleotide pairs are G-C pairs. In other embodiments, the nucleotide overhang may have 1 or 2 unpaired nucleotides, and in an exemplary embodiment the nucleotide overhang may be 5′-GC-3′. In another embodiment, the nucleotide overhang is on the 3′-end of the antisense strand.

Thus, an siNA molecule can include monomers which have been modified so as to inhibit degradation, e.g., by nucleases, e.g., endonucleases or exonucleases, found in the body of a subject. These monomers are referred to herein as NRMs, or Nuclease Resistance promoting Monomers or modifications. In some cases these modifications will modulate other properties of the siNA agent as well, e.g., the ability to interact with a protein, e.g., a transport protein, e.g., serum albumin, or a member of the RISC, or the ability of the first and second sequences to form a duplex with one another or to form a duplex with another sequence, e.g., a target molecule.

While not wishing to be bound by theory, it is believed that modifications of the sugar, base, and/or phosphate backbone in an siNA agent can enhance endonuclease and exonuclease resistance, and can enhance interactions with transporter proteins and one or more of the functional components of the RISC complex. In some embodiments, the modification may increase exonuclease and endonuclease resistance and thus prolong the half-life of the siNA agent prior to interaction with the RISC complex, but at the same time does not render the siNA agent inactive with respect to its intended activity as a target mRNA cleavage directing agent. Again, while not wishing to be bound by any theory, it is believed that placement of the modifications at or near the 3′ and/or 5′-end of antisense strands can result in siNA agents that meet the preferred nuclease resistance criteria delineated above.

Modifications that can be useful for producing siNA agents that exhibit the nuclease resistance criteria delineated above may include one or more of the following chemical and/or stereochemical modifications of the sugar, base, and/or phosphate backbone, it being understood that the art discloses other methods as well than can achieve the same result:

(i) chiral (Sp) thioates. An NRM may include nucleotide dimers with an enriched or pure for a particular chiral form of a modified phosphate group containing a heteroatom at the nonbridging position, e.g., Sp or Rp, at the position X, where this is the position normally occupied by the oxygen. The atom at X can also be S, Se, Nr₂, or Br₃. When X is S, enriched or chirally pure Sp linkage is preferred. Enriched means at least 70, 80, 90, 95, or 99% of the preferred form.

(ii) attachment of one or more cationic groups to the sugar, base, and/or the phosphorus atom of a phosphate or modified phosphate backbone moiety. In some embodiments, the may include monomers at the terminal position derivatized at a cationic group. As the 5′-end of an antisense sequence should have a terminal—OH or phosphate group this NRM is preferably not used at the 5′-end of an antisense sequence. The group should preferably be attached at a position on the base which minimizes interference with H bond formation and hybridization, e.g., away form the face which interacts with the complementary base on the other strand, e.g, at the 5′ position of a pyrimidine or a 7-position of a purine.

(iii) nonphosphate linkages at the termini. In some embodiments, the NRMs include Non-phosphate linkages, e.g., a linkage of 4 atoms which confers greater resistance to cleavage than does a phosphate bond. Examples include 3′CH₂—NCH₃—O—CH₂-5′ and 3′CH₂—NH—(O.dbd.)-CH₂-5′;

(iv) 3′-bridging thiophosphates and 5′-bridging thiophosphates. In certain embodiments, the NRM's can included these structures;

(v) L-RNA, 2′-5′ linkages, inverted linkages, a-nucleosides. In certain embodiments, the NRM's include: L nucleosides and dimeric nucleotides derived from L-nucleosides; 2′-5′ phosphate, non-phosphate and modified phosphate linkages (e.g., thiophosphates, phosphoramidates and boronophosphates); dimers having inverted linkages, e.g., 3′-3′ or 5′-5′ linkages; monomers having an alpha linkage at the 1′ site on the sugar, e.g., the structures described herein having an alpha linkage;

(vi) conjugate groups. In certain embodiments, the NRM's can include, e.g., a targeting moiety or a conjugated ligand described herein conjugated with the monomer, e.g., through the sugar, base, or backbone;

(vi) abasic linkages. In certain embodiments, the NRM's can include an abasic monomer, e.g., an abasic monomer as described herein (e.g., a nucleobaseless monomer); an aromatic or heterocyclic or polyheterocyclic aromatic monomer as described herein; and

(vii) 5′-phosphonates and 5′-phosphate prodrugs. In certain embodiments, the NRM's include monomers, preferably at the terminal position, e.g., the 5′ position, in which one or more atoms of the phosphate group is derivatized with a protecting group, which protecting group or groups, are removed as a result of the action of a component in the subject's body, e.g., a carboxyesterase or an enzyme present in the subject's body. For example, a phosphate prodrug in which a carboxy esterase cleaves the protected molecule resulting in the production of a thioate anion which attacks a carbon adjacent to the 0 of a phosphate and resulting in the production of an unprotected phosphate.

“Ligand”, as used herein, means a molecule that specifically binds to a second molecule, typically a polypeptide or portion thereof, such as a carbohydrate moiety, through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity (e.g., agonists or antagonists), the term as used herein applies more generally.

One or more different NRM modifications can be introduced into an siNA agent or into a sequence of an siRNA agent. An NRM modification can be used more than once in a sequence or in an siRNA agent. As some NRMs interfere with hybridization the total number incorporated, should be such that acceptable levels of siNA agent duplex formation are maintained.

In some embodiments NRM modifications are introduced into the terminal cleavage site or in the cleavage region of a sequence (a sense strand or sequence) which does not target a desired sequence or gene in the subject.

In most cases, the nuclease-resistance promoting modifications will be distributed differently depending on whether the sequence will target a sequence in the subject (often referred to as an antisense sequence) or will not target a sequence in the subject (often referred to as a sense sequence). If a sequence is to target a sequence in the subject, modifications which interfere with or inhibit endonuclease cleavage should not be inserted in the region which is subject to RISC mediated cleavage, e.g., the cleavage site or the cleavage region (As described in Elbashir et al., 2001, Genes and Dev. 15:188). Cleavage of the target occurs about in the middle of a 20 or 21 nucleotide guide RNA strand, or about 10 or 11 nucleotides upstream of the first nucleotide which is complementary to the guide sequence. As used herein cleavage site refers to the nucleotide on either side of the cleavage site, on the target or on the iRNA agent strand which hybridizes to it. Cleavage region means a nucleotide with 1, 2, or 3 nucleotides of the cleave site, in either direction.)

Such modifications can be introduced into the terminal regions, e.g., at the terminal position or with 2, 3, 4, or 5 positions of the terminus, of a sequence which targets or a sequence which does not target a sequence in the subject.

### VI. THERAPEUTIC USE

Mice without p21 develop certain types of cancer after a long latency period (Martin-Caballero et al., 2001, Cancer Res. 61:6234-6238). Loss of p21 in mice with disruptions in other cancer-associated genes also accelerates tumorigenesis (Yang et al., 2001, Cancer Res. 61: 565-569; Adnane et al., 2000, Oncogene 19:5338-5347; Bearss et al., 2002, Cancer Res. 62:2077-2084). These observations indicate a role for p21 in tumor suppression. Attenuation of p21 function in cancer cells may make cancer agents more effective (Weiss et al., Cancer Cell 2003, 4:425-429). Alternatively, there may be situations where it is desirable to accelerate cell cycle progression by increasing p21 function. For example, it may be desirable to accelerate cell cycle progression for wound healing or cell culture purposes (for example, to grow skin grafts in vitro). Therefore, identification of miRNAs that inhibit or accelerate cell cycle progression via p21 and other cell cycle regulatory genes (NKIRAS1, LIMK1, MAPRE3, RNH1, MAPK1) may be useful for treatment of patients, particularly cancer patients.

Examples of cancers that can be treated using the methods of the invention include, but are not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer. In general, an effective amount of the one or more compositions of the invention for treating cancer will be that amount necessary to inhibit mammalian cancer cell proliferation in situ. Those of ordinary skill in the art are well-schooled in the art of evaluating effective amounts of anti-cancer agents.

In some cases, treatment methods may be combined with known cancer treatment methods. The term “cancer treatment” as used herein, may include, but is not limited to, chemotherapy, radiotherapy, adjuvant therapy, surgery, or any combination of these and/or other methods. Particular forms of cancer treatment may vary, for instance, depending on the subject being treated. Examples include, but are not limited to, dosages, timing of administration, duration of treatment, etc. One of ordinary skill in the medical arts can determine an appropriate cancer treatment for a subject.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered (e.g., RNA, DNA or protein complex thereof) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16 26); biodegradable polymers, such as poly (DL-lactide-coglycolide) microspheres for sustained release delivery after implantation (Emerich, D F et al, 1999, Cell Transplant, 8, 47 58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941949, 1999). Other non-limiting examples of delivery strategies, including CNS delivery of the nucleic acid molecules of the instant invention include material described in Boado et al., 1998, J. Pharm. Sci., 87, 1308 1315; Tyler et al, 1999, FEBS Lett., 421, 280 284; Pardridge et al., 1995, PNAS USA., 92, 5592 5596; Boado, 1995, Adv. Drug Delivery Rev., 15, 73 107; Aldrian-Herrada et al., 1998, Nucleic Acids Res., 26, 4910 4916; and Tyler et al., 1999, PNAS USA., 96, 7053 7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601 2627; Ishiwata et al., Chem. Pharm. Bull. 1995, 43, 1005 1011). Such liposomes have been shown to accumulate selectively in tumors, presumably by extravasation and capture in the neovascularized target tissues (Lasic et al., Science 1995, 267, 1275 1276; Oku et al., 1995, Biochim. Biophys. Acta, 1238, 86 90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864 24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

### EXAMPLES

Examples are provided below to further illustrate different features and advantages of the present invention. The examples also illustrate useful methodology for practicing the invention. These examples do not limit the claimed invention.

### Materials and Methods:

Functional annotation of microRNAs. microRNA levels were measured in a collection of tumors and adjacent non-involved tissues that were obtained from between 50 to 70 patients for five different solid tumors (breast, colon, kidney, gastric, and lung). The tissue blocks were obtained from Genomics Collaborative, Inc (Laurel, Md.). Each sample was pulverized and split into two tubes, one of which was used for RNA extraction by use of an RNEasy kit (Qiagen, Valencia, Calif.). Purified total RNA samples were profiled on 25K Human Agilent microarrays. Samples were hybridized against pooled normal samples from the same tissue. Some of the remaining tumor RNA crude extracts, before purification on RNEasy columns, were used for miRNA analysis as described (Raymond et al., 2005, RNA 11: 1737-44). We looked at microRNAs in the signature of 10 or more tumor samples and annotated sets of ≧100 mRNAs correlating with microRNAs at r>0.4 or r<−0.4.

Microarray analysis. HCT116 Dicer^{ex5} cells were transfected in 6-well plates, and RNA was isolated 10 hr after transfection. Microarray analysis was performed as described previously (Jackson et al., 2003, Nat. Biotechnol. 21:635-7).

microRNA, mRNA and protein levels. microRNA levels were determined with the SuperScript III SYBR Green One-Step qRT-PCR system (Invitrogen, Carlsbad, Calif.). p21 mRNA levels were measured by qRT-PCR on an Applied Biosystems instrument. Protein levels were measured with antibodies against p21/CDKN1A (Cell Signaling Technology, Danvers, Mass.) in immunohistochemistry analysis in accordance with the manufacturer's instructions. Anti-HSP70 antibodies (Santa Cruz Biotechnology, Santa Cruz, Calif.) were used to test for equal loading.

Cell cycle analysis. Human mammary epithelial cells (HMECs) immortalized by stable integration of human telomerase (hTERT) (Smith et al., 2007, J. Biol. Chem. 282:2135-43) were obtained from J. Roberts (Fred Hutchinson Cancer Research Center, Seattle, Wash.) and were used in all experiments unless indicated otherwise. Cells expressing an inducible p21 shRNA were generated by lentiviral integration of a doxycyclin-responsive p21 shRNA construct into HMECs. Tumor-derived human color cancer cell lines, HCT116p21+′+ and HCT116p21l—, were obtained from B. Vogelstein (Johns Hopkins University School of Medicine, Baltimore, Md.). HCT116 Dicer^{ex5} cells were previously described (Bentwich et al., 2005, Nat. Genet. 37:766-70).

RNA duplexes corresponding to mature miRNAs were designed as previously described (Lim et al., 2005, Nature 433:769-73). miRNA mimics (10 nM), LNA anti-miR molecules (50 nM, Exiqon, Woburn, Mass.), and 2′-O-methyl anti-miR (Sigma-Proligo) were transfected using Lipofectamine RNAiMax (Invitrogen, Carlsbad, Calif.). Unless otherwise indicated, the anti-miR is LNA-modified. The anti-miR molecules are single stranded nucleotide sequences that are perfect complements of their target miRNAs. For p21 siRNA mediated knockdown, three siRNAs (obtained from Sigma-Proligo) designed with an algorithm that increases silencing efficiency and decreases off-target effects (Jackson et al, 2003, Nat. Biotechnol. 21:635-7) were pooled and transfected at 25 nM of each duplex. Subsequently, the siRNA pools were deconvolved and each siRNA was transfected at 25 nM, unless otherwise indicated. Where indicated, Nocodazole (100 ng ml⁻¹, Sigma Aldrich) was added 24 hr after transfection for 16-48 hr. Doxorubicin (500 nM) was added for 48 hr. Cell cycle distributions were measured by staining with propidium iodide as described (Linsley et al., 2007, Mol. Cell. Biol. 27:2240-52), followed by analysis on a FACSCalibur flow cytometer (Beckton Dickson). Data were analyzed with FlowJo software (Tree Star, Ashland, Oreg.).

For BrdU-incorporation analysis, 48 h after transfection, HMECs were pulsed with BrdU for 1 h (BD Bioscience). Fixed cells were stained with FITC-conjugated anti-BrdU antibody and the DNA dye 7-amino-actinomycin D (7-AAD).

miR-106b-mediated suppression reporter analysis. The 3′UTR from human p21/CDKN1A was amplified from human genomic DNA (Roche) and cloned into a vector containing the luciferase ORF (pMIR, Ambion, Austin, Tex.; pSGG_{—}3′UTR, SwitchGear Genomics). Seed regions were mutated to remove complementarity to the miR-106b by using the Quickchange II XL Mutagenesis Kit (Stratagene, La Jolla, Calif.). HCT116 Dicer^{ex5} cells were co-transfected with reporter construct and miRNAs using Lipofectamine 2000 (Invitrogen, Carlsbad, Calif.). pRL (Promega, Madison, Wis.) was transfected as a normalization control. Cells were lysed 24 hr after transfection, and ratios between firefly luciferase and Renilla luciferase activity were measured with a dual luciferase assay (Promega, Madison, Wis.).

### Example 1

### miR-106b Correlates with Cell Cycle Annotation and is Overexpressed in Tumor Samples

A number of miRNAs were functionally classified by correlating their expression levels in a set of human tumor and adjacent normal tissue samples with the expression of mRNA transcripts. The correlated mRNA transcripts were annotated with Gene Ontology (G0) Biological Processes terms (Ashburner et al., 2000, Nat. Genet. 25:25-29). Transcripts whose expression in vivo is correlated with expression of individual miRNAs may be enriched for transcripts characteristic of pathways regulated by the miRNAs.

FIG. 1A depicts a heat map of the expectation (E-value) for enrichment for G0 Biological Process terms in sets of transcripts that were positively correlated with the given miRNAs. Several miRNAs with known functions were correlated with transcripts with the expected annotation. miR-133b (and miR-1 and miR-133a, data not shown) levels correlated with transcripts annotated as being associated with muscle development, as would be expected for miRNAs that are specifically expressed in the muscle and that regulate muscle cell proliferation and differentiation (Chen et al, 2006, Nat. Genet. 38:228-33). miR-155 (BIC, B-cell integration cluster), a leukemogenic miRNA (Kluiver et al., 2005, J. Pathol. 207:243-249; E is et al, 2005, Proc. Natl. Acad. Sci. USA 102:3627-32), levels correlated with transcripts annotated to be involved in immune response. It is interesting to note that this oncogenic miRNA is not correlated with cell cycle related terms, suggesting that high levels of miR-155 in tumor samples may disrupt some other cellular processes that prevent tumorigenesis, rather than cell cycle progression.

Several members of the miR-106b family were correlated with cell cycle related terms (see FIG. 1A). miR-106b, miR-106a, miR-20, and miR-17-5p were correlated with DNA replication and mitosis, whereas miR-93 was correlated with DNA replication. These observations suggest that the miR-106b family regulates cell cycle progression and that the oncogenic potential of the miRNAs in this family is elicited by direct effects on cell division.

The positive correlation of the miR-106b family with cell-cycle terms predicts that the expression levels of these miRNAs are elevated in highly proliferative tissues and cancer samples. Previously, it was shown that miRNAs derived from the miR-17-92 locus are overexpressed in B-cell lymphomas that have chromosomal amplifications of this locus (He et al., 2005, Nature 435:828-33) and in some lung cancer cell lines (Hayashita et al., 2005, Cancer Res. 65:9628-32). We extended these findings in an unbiased approach and compared a panel of tumor samples from several tissues. We found that the miR-106b family is overexpressed in a tumor samples from five different tissues as compared to adjacent, normal samples (FIG. 1C). The positive correlation with cell-cycle terms and the high levels in proliferative tissues suggest that the miR-106b family may contribute to tumor growth by promoting cell proliferation.

### Example 2

### miR-106b Affects Cell Cycle Progression

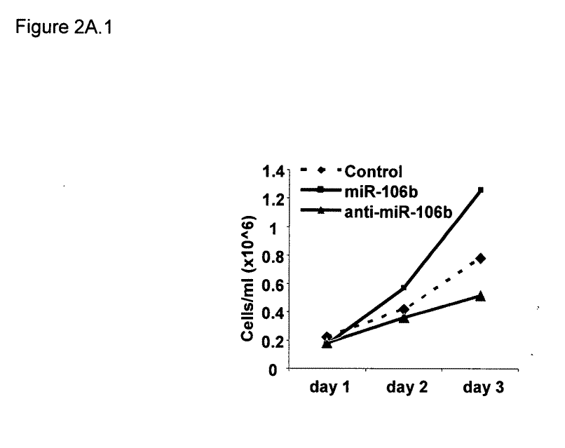

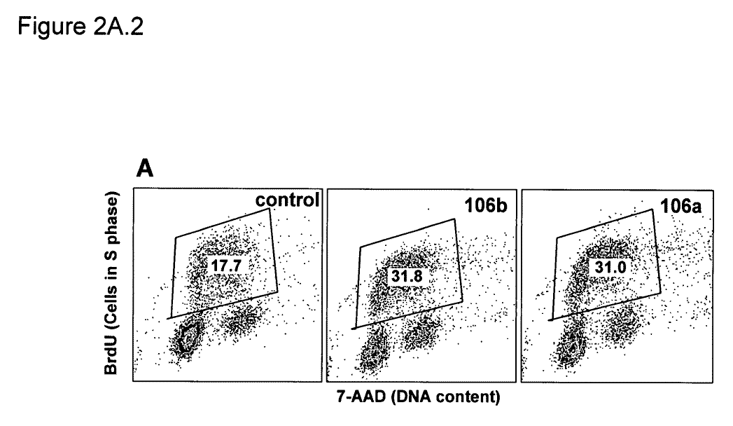

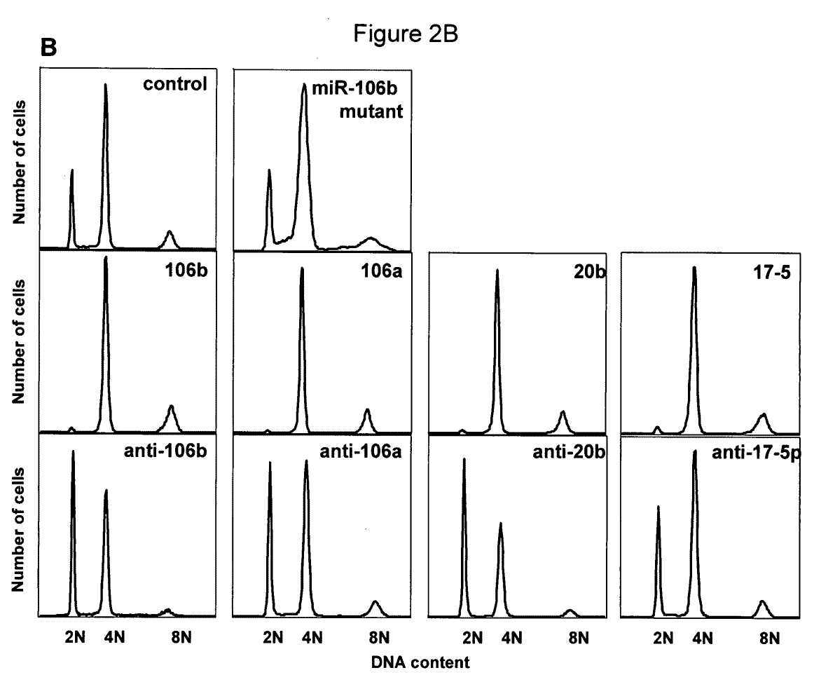

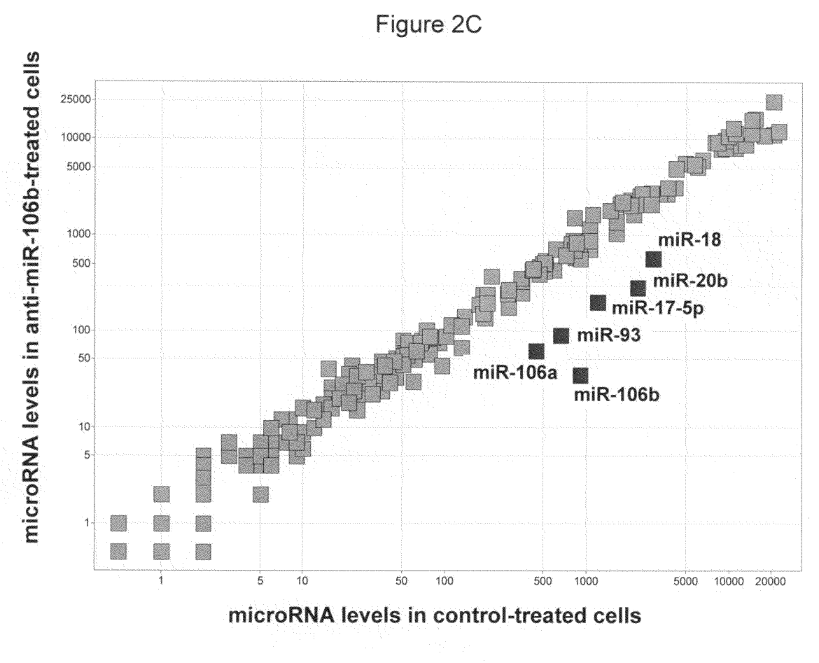

To directly test whether the miR-106b family accelerates cell cycle progression, we performed gain-of-function or loss-of-function experiments. Synthetic RNA duplexes, designed to mimic the miRNAs, or anti-miRs, to inhibit the microRNAs, were transfected into asynchronously-growing cells. As shown in FIG. 2A.1, a miR-106 duplex promoted cell division compared with a control duplex, whereas anti-miR-106b retarded cell division.

Cell cycle profiles were analyzed by flow cytometry. Overexpression of miR-106b, miR-106a, miR-20b and miR-17-5p resulted in an increase in the S phase population as measured by propidium iodide staining (data not shown) and BrdU incorporation (FIG. 2A.2). Following a one hour pulse of BrdU, control-treated cells had 17.7% of cells in S phase, whereas miR-106b- and miR-106a-treated cultures had 31.8% and 31.0% S-phase cells, respectively. No increase in the S-phase population was observed with miR-93 and miR-372 and with miRNAs with unrelated seed regions (miR-18, miR-19, miR-92, data not shown), suggesting that these 106b-family members may not have cell proliferative functions.

The accumulation of cells in S phase by the miR-106b-family suggests that these miRNAs either accelerate progression from G1 to S or retard progression through S phase. To distinguish between these possibilities, we treated cells with the microtubule depolymerizing drug, nocodazole to block cell cycle progression at G2/M (FIG. 2B). Retardation of S phase is predicted to result in S phase accumulation following nocodazole treatment, whereas acceleration of the G1 to S transition will lead to more complete accumulation in G2/M. As shown in FIG. 2B (top), control-treated cells accumulated in G2/M (with 4N DNA content), with a residual G6 population (2N). miR-106b, miR-106a, miR-20b, and miR-17-5p mimics reduced the G1 population, indicating that these miRNAs drive cells out of G1 and accelerate the nocodazole-mediated accumulation in G2/M (FIG. 2B, middle panel). This phenotype is dependent on seed-region complementarity, as mutation nucleotides 2 and 3 in miR-106b abrogated the effect (FIG. 2B, top right)

During the course of this study, an alternative miR-93 was cloned, containing an additional base at the 5′ end (referred to as miR-93_{—}2 (SEQ ID NO:7); see FIG. 6A; Landgraf et al., 2007, Cell 129:1401-1414), putting it in the register with miR-106a, miR-106b, miR-17-5-p, and miR-20. miR-372 was not cloned in the Landgraf study, likely due to its low expression in somatic tissues. In the present study, phenotypes of both the original miR-93 (SEQ ID NO:18) and miR-372 (SEQ ID NO:19) sequences and of sequences containing an additional base at the 5′ end (miR-93_{—}2 (SEQ ID NO:7) and miR-372_{—}2 (SEQ ID NO:6), respectively, see FIG. 6A) were examined to determine the dependence of miRNA sequence on the base composition. The original miR-93 (SEQ ID NO: 18) and miR-372 (SEQ ID NO: 19) did not show the miR-106b phenotype of lower G1 population, whereas the sequences with the additional base at the 5′ end (shifting the “AAAGUG” seed region (SEQ ID NO:8) in alignment with positions 2-8), miR-93_{—}2 (SEQ ID NO:7) and miR-372_{—}2 (SEQ ID NO:6), had more subtle effects on this phenotype (FIG. 6B). This data suggests that positions 2-8 of the miR-106b seed region (SEQ ID NO:8) mediate the cell cycle phenotype of miR-106b. Other miR-106b family members may show this phenotype as nucleotide variations in the miR seed region are discovered. We concentrated on the four miRNAs with the most robust phenotypes (miR-106b (SEQ ID NO:1), miR-106a (SEQ ID NO:2), miR-17-5p (SEQ ID NO:5), and miR-20b (SEQ ID NO:4)) for further study. It should be noted that miR-20a (SEQ ID NO:3) (which has the miR-106b seed region at positions 2-8) also demonstrated the same cell cycle progression phenotype as miR-106b, miR-106a, miR-17-5-p, and miR-20b (data not shown).

In summary, these experiments revealed a functional conservation within the miR-106b family as positive regulators of the G1-to-S transition.

### Example 3

### Anti-miR-106b Slows Cell Cycle Progression by Targeting Multiple Family Members

Acceleration of cell cycle progression by the miR-106b family may reflect the intrinsic function of the miRNAs, or an ectopic gain-of-function as a result of non-physiological levels. To identify the cellular function of the miR-106b family, we used LNA-conjugated anti-miRs to suppress the endogenous miRNAs.

If the miR-106b family is required for progression from G1 to S, then a decrease of mature miRNA levels will result in slower cell cycle progression and in accumulation of cells in G1. We found that anti-miR-106b (SEQ ID NO:9), anti-miR-106a (SEQ ID NO:10), anti-miR-20b (SEQ ID NO:12), and anti-miR-17-5p (SEQ ID NO:13) had the reverse effect from the miRNA overexpressions and resulted in an accumulation of cells in G1 (2N) upon treatment with nocodazole (FIG. 2B, bottom panels). Even after prolonged exposure to nocodazole (72 hr), a subpopulation of ˜20% of cells remained blocked in G1 (2N), suggesting that the miR-106b family is required for the G1-to-S transition (FIG. 7). Similarly, 2′-O-methyl modified anti-miR-106b (SEQ ID NO:9) also reversed the effect of miR-106b overexpression (FIG. 9), indicating that this miR-106b inhibition may be achieved by different means.

To confirm that the phenotypes of the LNA-conjugated anti-miRs are due to engagement of their miRNA targets, we performed miRNA expression profiling on anti-miR-treated samples (Raymond et al., 2005, RNA 11:1737-44). FIG. 2C shows that treatment with anti-miR-106b resulted in specific knockdown of all miR-106b family members assayed (the assay for miR-372 failed in this platform, whereas miR-520, miR-526b* and miR-519 were not included). Similar results were observed with anti-miRs against the other family members (data not shown). Thus, treatment with anti-miRs results in specific knockdown of miRNAs dependent on seed-region similarity.

To test phenotypically whether anti-miRs target multiple family members, we performed mixing experiments. Cells initially transfected with a miRNA, were subsequently transfected with an anti-miR, and cell cycle profiles were analyzed. As miR-106b family leads to accumulation of cells in S phase (FIG. 2A.2), the percent of cells in S phase was recorded as a reliable metric for miRNA effect. As expected, treatment with miR-106b, miR-106a, miR-20 and miR-17-5p followed by control duplex resulted in an accumulation of S-phase cells (26-38% vs. 15% in control-miR-treated cells, Table 1A). anti-miR-106b (SEQ ID NO:9) reversed this effect regardless of the miRNA used in the initial treatment resulting in 14-20% of cells in S phase (Table 1A). Similarly, anti-miR-106b (SEQ ID NO:9), anti-miR-106a (SEQ ID NO:10), anti-miR-20 (SEQ ID NO:12), and anti-miR-17-5p (SEQ ID NO:13) reversed the miR-106b-mediated accumulation in S phase from 26% to 17-21% (Table 1B). Interestingly, anti-miR-372 (SEQ ID NO:16) had a less potent effect on miR-106b suggesting that seed-region identity at positions 2-8 is important for miRNA:anti-miR interactions. Thus, anti-miRs against one family member reverse the phenotypes of multiple microRNAs in the family, revealing that anti-miRs may not be exquisitely specific in targeting individual members of a miRNA family.

Table 1. anti-miRs reverse the accumulation of cells in S phase elicited by multiple miR-106b family members. (A). Cells were first transfected with the indicated miRNA, and subsequently with either a control duplex or with anti-miR-106b. Cells were harvested and cell cycle profiles were analyzed by flow cytometry. The numbers indicate percent of cells in S phase. miR-106b, miR-106a, miR-20 and miR-17-5p led to an accumulation of cells in S phase over the control duplex, whereas miR-93 does not show this phenotype. anti-miR-106b reverses the effect of miR-106b, miR-106a, miR-20 and miR-17-5p resulting in S phase percentages that are comparable to the control treated cells. (B). In a reciprocal experiment, cells were first transfected with miRNA-106b, and subsequently with either a control duplex or the indicated anti-miRs. Cells were harvested and cell cycle profiles were analyzed by flow cytometry. The numbers indicate percent of cells in S phase. The S phase accumulation elicited by miR-106b (top row, 26%), was reversed by each of anti-miR-106b, anti-miR-106a, anti-miR-20b and anti-miR-17-5p. The effect of anti-miR-372 was more subtle.

TABLE 1A

anti-miR

| microRNA | control | anti-miR-106b |
|---|---|---|
| control | 15 | 14 |
| miR-106b | 26 | 20 |
| miR-106a | 34 | 20 |
| miR-20b | 38 | 14 |
| miR-17-5p | 30 | 18 |
| miR-93 | 13 | 16 |

TABLE 1B

| | microRNA |
|---|---|
| anti-miR | miR-106b |
| control | 26 |
| anti-miR-106b | 20 |
| anti-miR-106a | 18 |
| anti-miR-20b | 17 |
| anti-miR-17-5p | 21 |
| anti-miR-372 | 23 |

In summary, anti-miRs against miR-106b family lead to slowing of cell cycle progression at the G1-to-S transition. This phenotype is likely a result of knockdown of multiple miRNAs in the miR-106b family. Therefore, the G1-to-S function for the miR-106b family inferred by the gain-of-function analysis is the endogenous cellular mechanism for these miRNAs.

### Example 4

### Cell-Cycle Targets of miR-106b Family

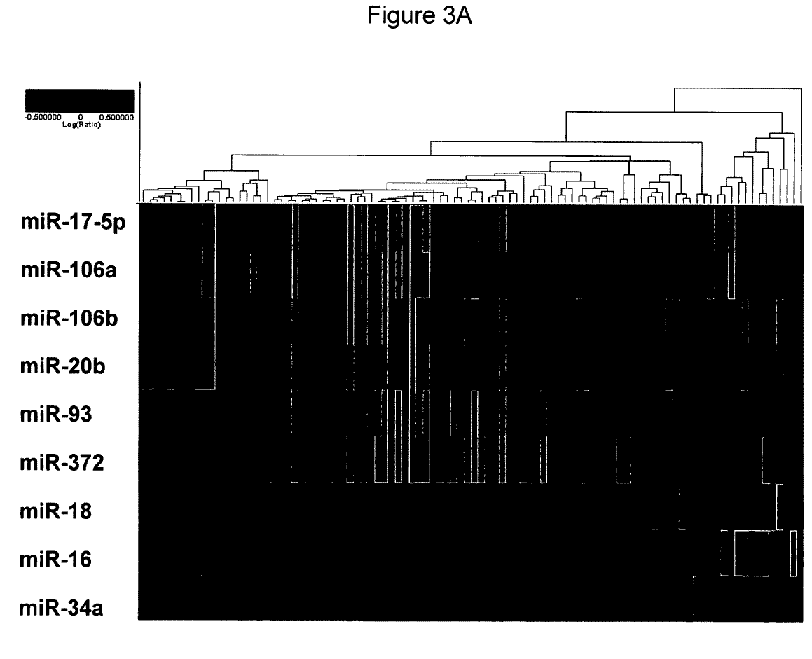

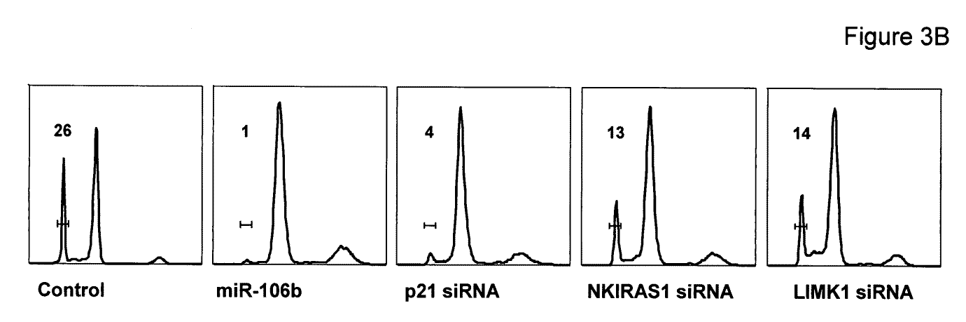

The positive effects of the miR-106b family on cell cycle progression are likely a result of downregulation of gene(s) that negatively regulate cell cycle progression. To identify the targets of the miR-106b family, we performed mRNA expression profiling after transfection of microRNA mimics. We concentrated on genes that were downregulated at an early time point (10 h post-transfection of HCT116 Dicer^{ex5}) to enrich for direct targets. Finally, we filtered the genes for those containing miR-106b family hexamers (the miR-106b seed region of positions 2-8 (SEQ ID NO:8)) in their 3′UTRs. The resulting of set of 103 transcripts that were down-regulated by miR-106b, miR-106a, miR-20b, and miR-17-5p at 10 hrs after transfection and contained miR-106b-family seed region hexamers in their 3′ UTR is listed in Table 2; a subset of 96 genes is shown in a heatmap in FIG. 3A (Contig identifiers for some transcripts are described in Ewing et al., 25:232-234 and available on www.phrap.org). The down regulation signatures of miR-93 and miR-372 were largely overlapping with the other miR-106b family members (17-5p, 106a, 106b, 20b), whereas miR-18 (a microRNA with one base pair mismatch in the seed region from the miR-106b family, FIG. 1B), miR-16, and miR-34a (the latter two are unrelated microRNAs with cell-cycle functions) did not affect the majority of the miR-106b-family regulated genes (see FIG. 3A). Coordinate regulation of these genes is the likely mechanism by which the miR-106b family regulates cellular processes, including the G1-to-S transition.

TABLE 2
103 gene targets of miR-106b family

| Transcript Identifier | Gene Name |
|---|---|
| NM_020792 | AADACL1 |
| NM_016248 | AKAP11 |
| NM_022476 | AKTIP |
| NM_016374 | ARID4B |
| NM_014109 | ATAD2 |
| NM_030803 | ATG16L1 |
| Contig53819_RC | BRMS1L |
| NM_006806 | BTG3 |
| NM_145247 | C10orf78 |
| hCT401251.3 | C21orf25 |
| NM_018275 | C7orf43 |
| NM_017998 | C9orf40 |
| NM_032012 | C9orf5 |
| NM_023925 | CAPRIN2 |
| NM_053056 | CCND1 |
| NM_017913 | CDC37L1 |
| NM_000389 | CDKN1A (p21) |
| NM_018132 | CENPQ |
| NM_021914 | CFL2 |
| NM_024692 | CLIP4 |
| NM_004898 | CLOCK |
| NM_024843 | CYBRD1 |
| NM_014764 | DAZAP2 |
| NM_032998 | DEDD |
| NM_004418 | DUSP2 |
| Contig48913_RC | E2F2 |
| NM_012199 | EIF2C1 |
| NM_004094 | EIF2S1 |
| NM_020390 | EIF5A2 |
| NM_021572 | ENPP5 |
| NM_024293 | FAM134A |
| NM_024792 | FAM57A |
| NM_182752 | FAM79A |
| NM_006712 | FASTK |
| NM_012161 | FBXL5 |
| NM_024513 | FYCO1 |
| NM_003506 | FZD6 |
| NM_002077 | GOLGA1 |
| NM_003272 | GPR137B |
| AL834372 | GPR137C |
| NM_012080 | HDHD1A |
| NM_181507 | HPS5 |
| Contig52427_RC | ITGB8 |
| NM_002227 | JAK1 |
| Contig56422_RC | KATNAL1 |
| NM_014774 | KIAA0494 |
| NM_014732 | KIAA0513 |
| NM_004522 | KIF5C |
| NM_005655 | KLF10 |
| NM_016357 | LIMA1 |
| NM_002314 | LIMK1 |
| NM_002745 | MAPK1 |
| NM_012326 | MAPRE3 |
| NM_014874 | MFN2 |
| NM_013446 | MKRN1 |
| NM_005955 | MTF1 |
| NM_000431 | MVK |
| NM_013262 | MYLIP |
| NM_017567 | NAGK |
| NM_018092 | NETO2 |
| NM_020345 | NKIRAS1 |
| NM_032235 | NPAS2 |
| NM_014778 | NUPL1 |
| NM_000297 | PKD2 |
| NM_006823 | PKIA |
| NM_002657 | PLAGL2 |
| NM_020353 | PLSCR4 |
| NM_000950 | PRRG1 |
| NM_024081 | PRRG4 |
| AK056651 | PURB |
| NM_020673 | RAB22A |
| NM_145313 | RASGEF1A |
| NM_020211 | RGMA |
| NM_002939 | RNH1 |
| NM_001034 | RRM2 |
| Contig55558_RC | SAR1B |
| Contig54946_RC | SENP1 |
| NM_004694 | SLC16A6 |
| AL122071 | SLC16A9 |
| NM_014585 | SLC40A1 |
| NM_173354 | SNF1LK |
| NM_013323 | SNX11 |
| NM_145251 | STYX |
| AB020689 | TBC1D9 |
| NM_017849 | TMEM127 |
| NM_032780 | TMEM25 |
| Contig53226_RC | TMEM64 |
| NM_020644 | TMEM9B |
| NM_021137 | TNFAIP1 |
| NM_003842 | TNFRSF10B |
| NM_014452 | TNFRSF21 |
| NM_018700 | TRIM36 |
| NM_007275 | TUSC2 |
| NM_022832 | USP46 |
| NM_025076 | UXS1 |
| Contig49273_RC | VANGL1 |
| NM_014872 | ZBTB5 |
| NM_006626 | ZBTB6 |
| NM_053023 | ZFP91 |
| NM_007324 | ZFYVE9 |
| NM_153695 | ZNF367 |
| Contig40903_RC | ZNF800 |
| NM_021035 | ZNFX1 |

This 103 gene miR-106b-family signature contains 14 genes annotated as “cell cycle” genes by G0 Biological Processes (listed in Table 3). This set of genes contains the likely relevant targets that mediate the cell-cycle phenotype of the miR-106b family. We reasoned that robust down-regulation of these genes should phenocopy miR-106b-family gain-of-function. Therefore, we tested whether siRNA-mediated knockdown leads to reduction in G1-phase cells upon treatment with nocodazole as seen for miR-106b family (Table 3 and FIG. 3B). We found that knockdown of six genes (p21/CDKN1A (SEQ ID NOs: 33,34), LIMK1 (SEQ ID NO:35), NKIRAS1 (SEQ ID NO:36), MAPRE3 (SEQ ID NO:37), RNH1 (SEQ ID NO:38), and MAPK1(SEQ ID NO:39)) phenocopied miR-106b-family gain-of-function, two genes had no effect on this phenotype, and six genes led to an accumulation of cells in G1 (FIG. 3B). Silencing of an additional 74 targets did not reveal any that strongly phenocopied miR-106b (data not shown).

To test whether the miR-106b-family cell-cycle phenotype is a result of coordinate regulation of these targets, we combined siRNAs targeting the top three genes (p21, LIMK1, and NKIRAS1) at suboptimal concentrations. Under these conditions, the siRNAs led to a partial gene knock down comparable to the miR-106b-family-mediated levels, and each siRNA alone had no effect on cell cycle progression. When combined, partial knockdown of the top three targets phenocopied miR-106b family gain-of-function, consistent with coordinate regulation of several targets (data not shown).

Strikingly, one target, p21/CDKN1A, had consistently stronger effect on cell cycle progression and therefore stood out as a likely key target of the miR-106b family in the cell cycle phenotype. p21/CDKN1A is a known negative regulator of the G1-to-S transition (reviewed in Sherr and Roberts, 1999, Genes Dev.13:1501-12). Therefore, we explored the relationship between p21 and miR-106b further.

TABLE 3
Cell-cycle genes downreguiated by the miR-106b
family.

| | | |
|---|---|---|
| | % G1 in nocodazole by | |
| | siRNA poci or a repre- | |
| Gene | sentative single siRNA | SEQ ID NO: |

| | | |
|---|---|---|
| Luc | 25 | |
| miR-106b | 5 | |
| CDKN1A (p21) | 6 | 33,34 |
| NKIRAS1 | 6 | 36 |
| LIMK1 | 10 | 35 |
| MAPRE3 | 15 | 37 |
| RNH1 | 16 | 38 |
| MAPK1 | 17 | 39 |
| TUSC2 | 24 | 40 |
| BTG3 | 27 | 41 |
| ARID4B | 41 | 42 |
| RRM2 | 44 | 43 |
| KATNAL1 | 48 | 44 |
| CCND1 | 50 | 45 |
| CDC37L1 | 50 | 46 |
| SNF1LK | 53 | 47 |

### Example 5

### p21 is a Direct Target of miR-106b

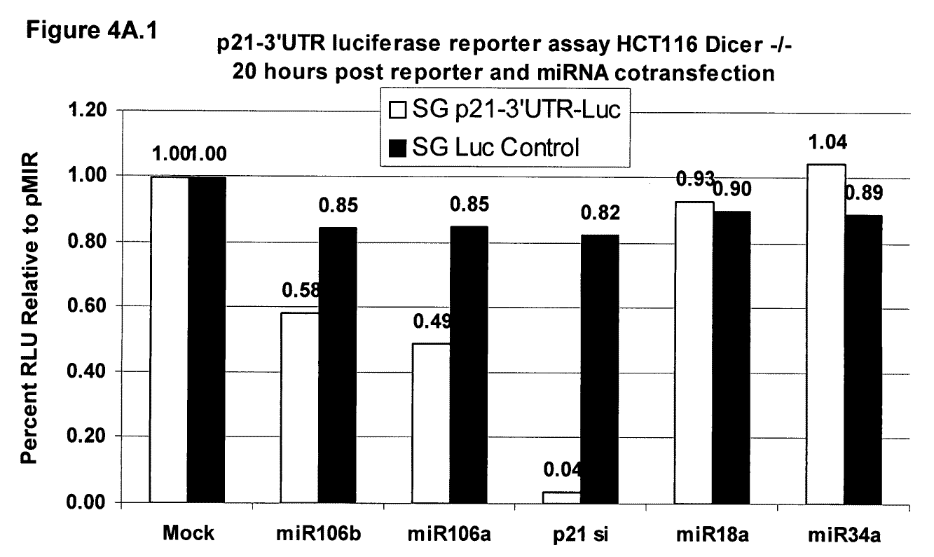

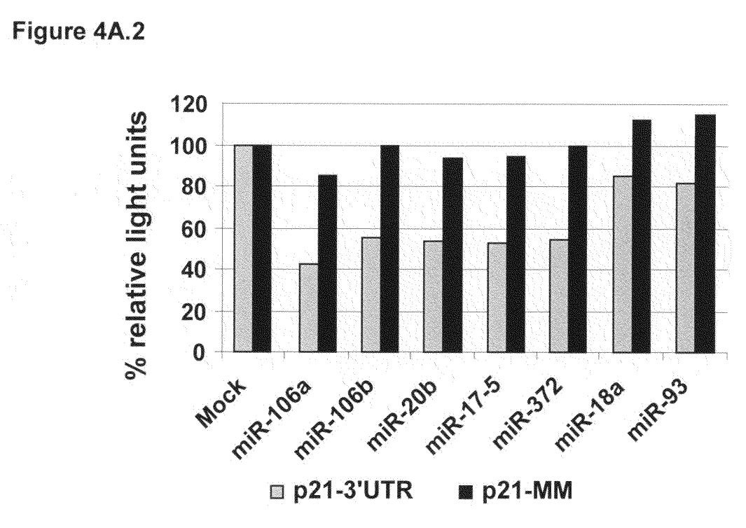

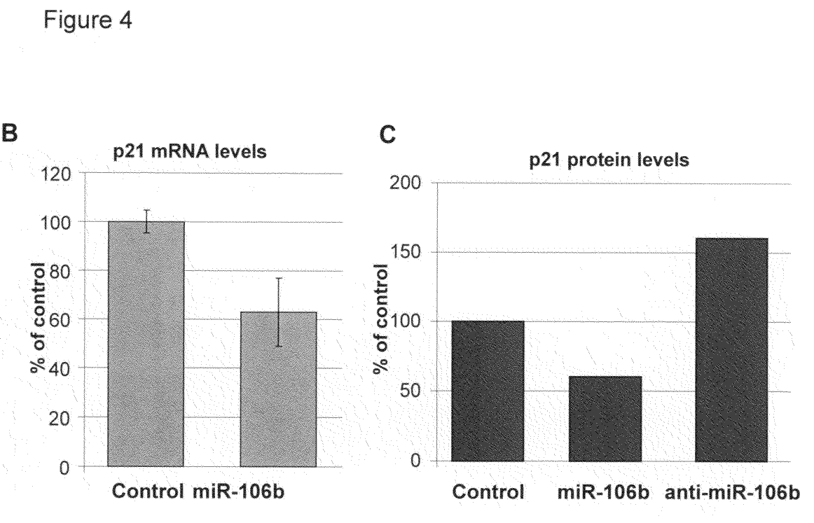

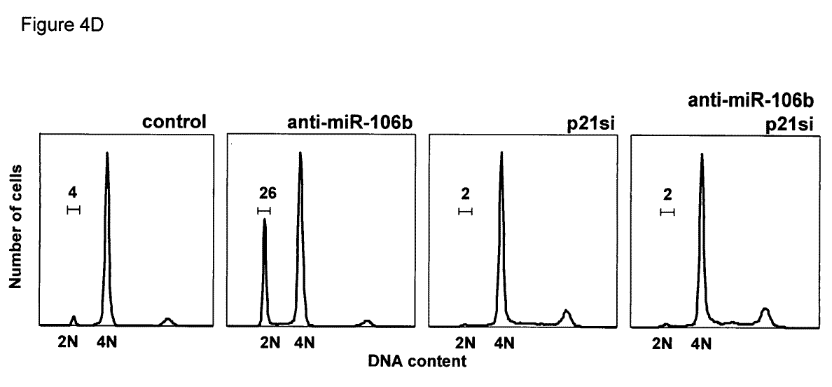

To test whether miR-106b directly affects p21 transcript levels, we performed in vitro Luciferase reporter assays. The p21/CDKN1A 3′UTR contains two hexamer complementary to the miR-106b-family seed region (SEQ ID NO:8). We found that transfection of several members of the miR-106b family resulted in downregulation of the luciferase reporter when it was followed by the p21 3′UTR (FIG. 4A.1). This effect depends upon the seed region hexamer complementarity in the p21 3′UTR, as mutations in these sequences rendered the reporter non-responsive to micro RNA transfection (see FIG. 4A.2, black bars). A p21-promoter-luciferase reporter did not respond to miR-106b transfection, ruling out indirect effects of miR-106b on p21 transcription (data not shown). Because all family members behaved comparably, we concentrated on miR-106b as a representative.

We next tested whether p21 mRNA and protein levels fluctuate upon miR-106b gain-of-function and knockdown. We observed a 38% reduction of p21 mRNA in miR-106b-overexpressing cells by qPCR analysis (FIG. 4B). p21 protein levels were reduced to 60% in miR-106b overexpressing cells and increased to 160% by anti-miR-106b as compared to control-treated cells (FIG. 4C). These results are consistent with p21 being a key target of miR-106b.

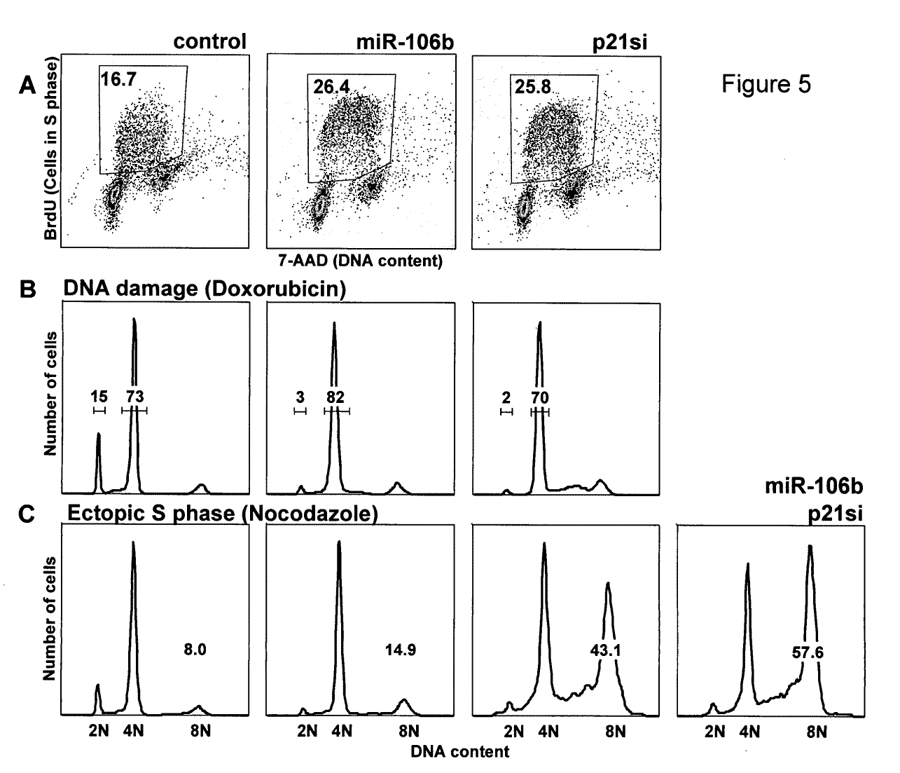

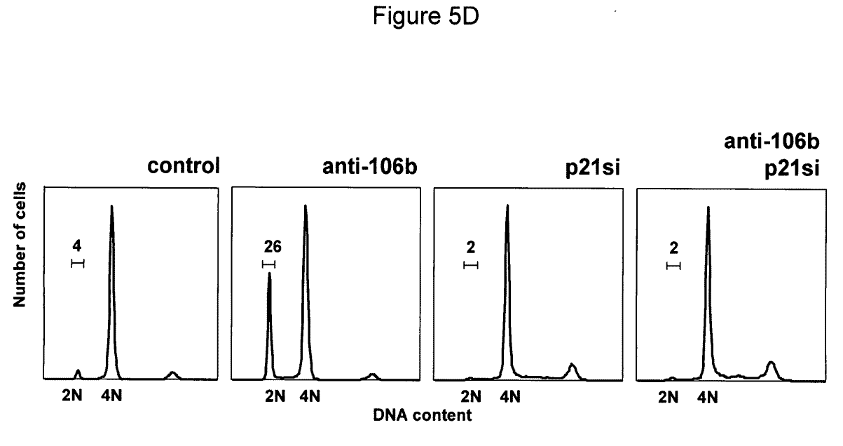

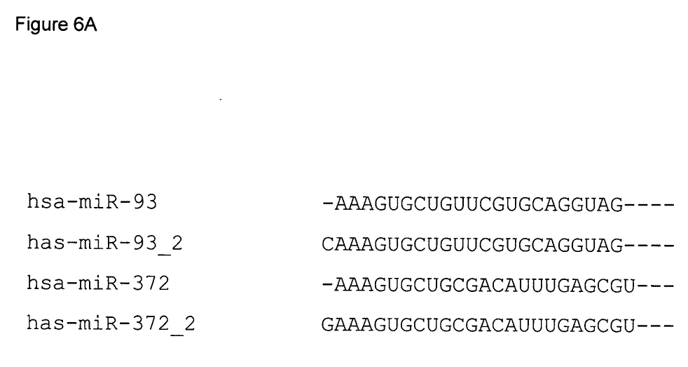

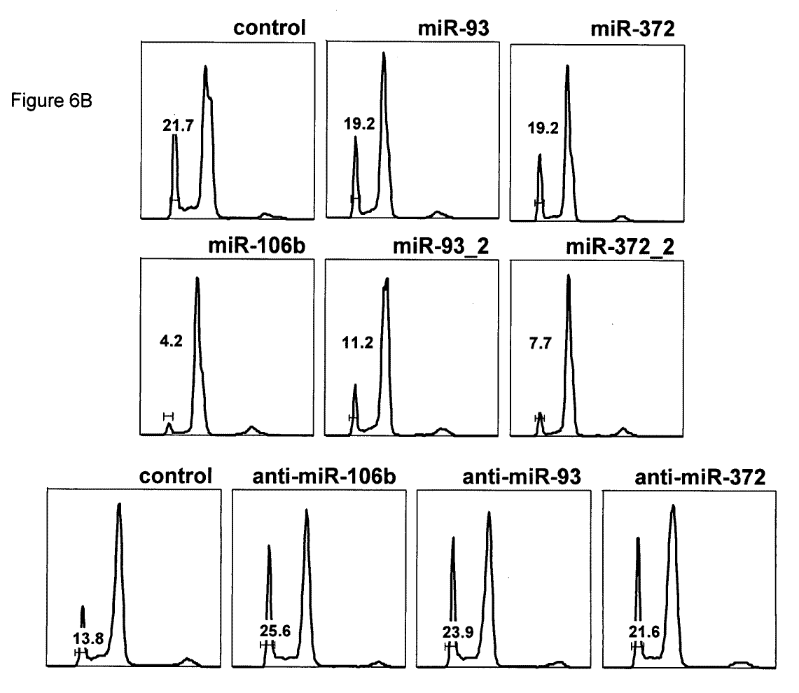

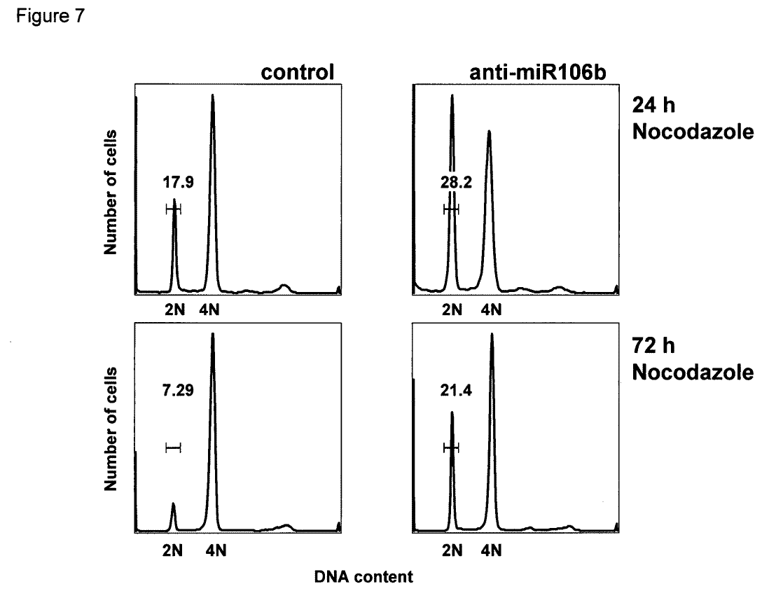

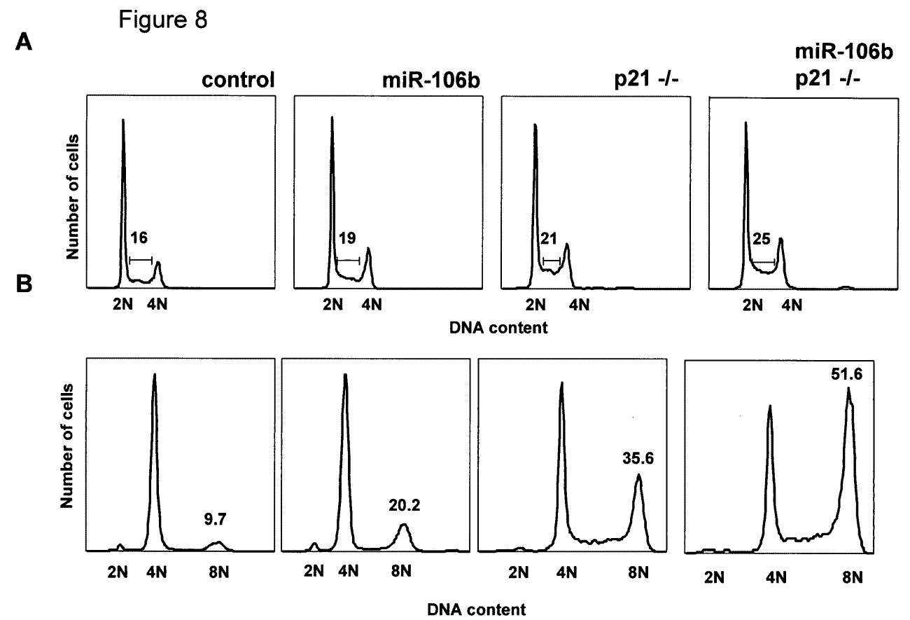

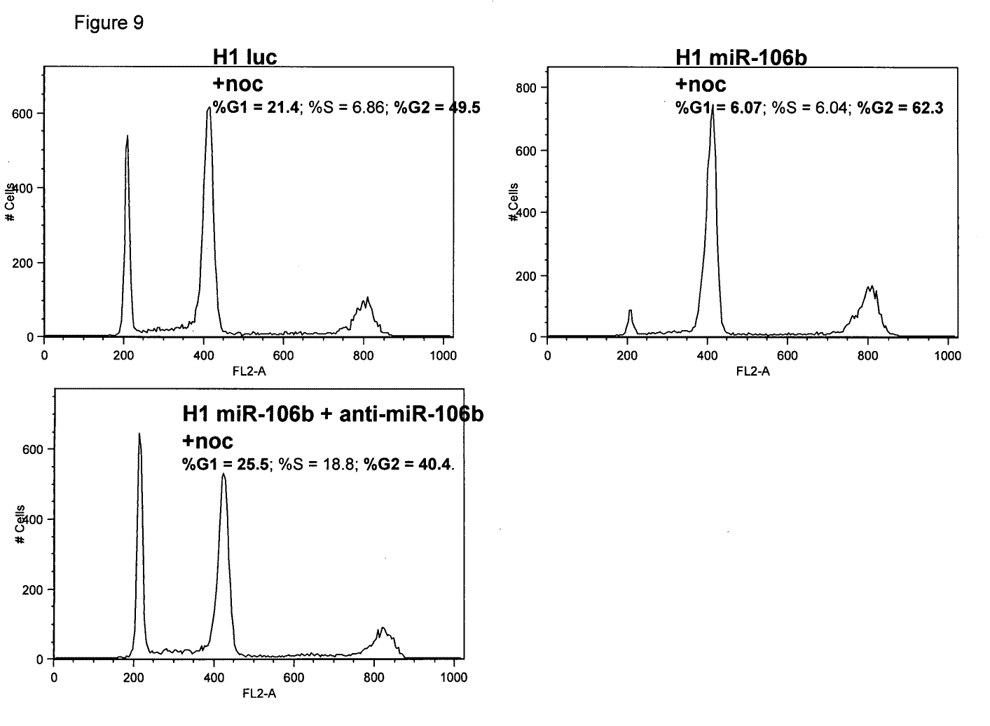

Physiologically-relevant targets of a microRNA should reflect the phenotypes of that microRNA. Knockdown of the target gene is expected to phenocopy gain-of-function of the microRNA. We used a pool of three siRNAs against p21 to knockdown the gene by 87% at the mRNA level. FIGS. 5A and 8A show that p21 knockdown resulted in S phase accumulation comparable to that observed by miR-106b gain-of-function. We obtained analogous results with HCT116 colon carcinoma cells deleted for p21 by homologous recombination (Waldman et al, 1995, Cancer Res. 55:5187-90) (FIG. 8). In addition, p21 knockdown resulted in reduction of the G1 population in nocodazole-treated cells, from 26% G1 in control-treated cells to 5% and 3% in miR-106b and p21 siRNA-treated cells, respectively. Therefore, p21 is likely one of the in vivo targets of miR-106b.

To further establish a functional connection between miR-106b and p21, we tested whether p21 is required for the anti-miR-106b phenotype. If the anti-miR-106b phenotype depends on increased p21 levels, then the absence of p21 should abrogate the effect. When we silenced p21 with an siRNA, anti-miR-106b no longer elicited an accumulation in G1 (FIG. 4D). This phenotype is not due to competition between p21 siRNA and anti-miR-106b, as similar results were obtained in HCT116 p21^{-/-} cells (data not shown). These results show that p21 is required for the anti-miR-106b phenotype and that the observed increase in p21 protein levels in anti-miR-106b-treated cells is not a secondary consequence of increased numbers of cells in G1.

### Example 6

### miR-106b Modulates the Checkpoint Functions of p21

p21 is the main downstream target of TP53 that transduces the TP53-dependent G1 block upon DNA damage (Waldman et al., 1995, Cancer Res. 55:5187-90) and is required to prevent nocodazole-treated cells from reentering an unscheduled round of DNA synthesis (Lanni and Jacks, 1998, Mol. Cell. Biol. 18:1055-64). Therefore, we investigated whether miR-106b gain-of-function phenocopies p21 loss for its inhibition of a robust G1-checkpoint.

Treatment with Doxorubicin, a DNA damage reagent, causes a dual cell cycle block in G1 and G2/M (FIG. 5B, left panel). The G1 block is dependent on p21 and TP53 (FIG. 5B, right panel, Waldman et al., 1995, Cancer Res. 55:5187-90)). We found that miR-106b gain-of-function overrides this block in a p21-proficient background (FIG. 5B, middle panel).

Cells treated with nocodazole for prolonged periods (≧48 hr) adopt to the G2/M block, flatten and are similar to G1 cells, with upregulated cyclin E (Lanni and Jacks, 1998, Mol. Cell. Biol. 18:1055-64). TP53 and p21 prevent adopted cells from entering an unscheduled S phase and accumulating as polyploid cells with 8N or greater DNA content due to endoreduplication (FIG. 5C) (Cross et al., 1995, Science 267:1353-6; Lanni and Jacks, 1998, Mol. Cell. Biol. 18:1055-64; Stewart et al., 1999, Mol. Cell. Biol. 19:205-15). We found that overexpression of miR-106b also led to an accumulation of 8N cells upon prolonged nocodazole block (FIG. 5C). The miR-106b phenotype (15% 8N) is less pronounced than that of p21 siRNA-mediated knockdown (43% 8N) likely because there is more p21 protein remaining in the microRNA-treated cells (FIG. 4B) as compared to siRNA-treated cells (data not shown). We observed greater accumulation of 8N cells in cultures overexpressing miR-106b and with p21 siRNA knockdown than in either condition alone, suggesting a synergistic effect (FIG. 5C). We also observed the endoreduplication phenotype in HCT116 cells overexpressing miR-106b and/or deleted for p21 (FIG. 8B). In summary, miR-106b overexpression overrides the cell cycle checkpoint established by the TP53-p21 pathway in G2/M-blocked cells.

Finally, we assayed the effects of anti-miR-106b in a p21-deficient background. If miR-106b acts through p21 then, upon downregulation of its substrate by alternate means, the effects of anti-miR-106b are expected to be lost. We found that p21 loss was epistatic to anti-miR-106b (FIG. 5D), suggesting that in a p21-deficient background, silencing of miR-106b no longer results in upregulation of p21 and to slowing of cell cycle progression.

1. A method of inhibiting proliferation of a cell comprising introducing an effective amount of a miR-specific inhibitor of at least one miR-106b family member into the cell.

2. The method of claim 1, wherein the cell is a mammalian cell.

3. The method of claim 1, wherein the cell is a cancer cell.

4. The method of claim 1, wherein the at least one miR-106b family member is selected from the group consisting of miR-106b, miR-106a, miR-20a, miR-20b, and miR-17-5p.

5. The method of claim 1, wherein the at least one miR-106b family member comprises miR-106b.

6. The method of claim 1, wherein the at least one miR-106b family member comprises miR-106a.

7. The method of claim 1, wherein the miR-specific inhibitor is selected from the group consisting of anti-miRs and target mimics.

8. The method of claim 1, wherein the miR-specific inhibitor comprises a nucleotide sequence of least 6 consecutive nucleotides that are complementary to the positions 2-8 of the seed region of said miR-106b family member, and has at least 50% complementarity to the rest of said miR-106b family member sequence, and wherein the miR-specific inhibitor of at least one miR-106b family member retards the G1-to-S transition.

9. The method of claim 8, wherein the miR-specific inhibitor of at least one miR-106b family member up-regulates p21.

10. The method of claim 8, wherein said miR-specific inhibitor has at least 60% complementarity to the rest of said miR-106b family member sequence.

11. The method of claim 8, wherein said miR-specific inhibitor has at least 70% complementarity to the rest of said miR-106b family member sequence.

12. The method of claim 8, wherein said miR-specific inhibitor has at least 80% complementarity to the rest of said miR-106b family member sequence.

13. The method of claim 8, wherein said miR-specific inhibitor has at least 90% complementarity to the rest of said miR-106b family member sequence.

14. The method of claim 8, wherein said miR-specific inhibitor is chemically modified on at least one nucleotide.

15. The method of claim 14, wherein said chemical modification comprises LNA.

16. The method of claim 14, wherein said chemical modification comprises 2′-O-methyl.

17. The method of claim 5, wherein the miR-specific inhibitor comprises a polynucleic acid molecule that is essentially complementary to miR-106b.

18. The method of claim 5, wherein the miR-specific inhibitor comprises a polynucleic acid molecule that is 100% complementary to miR-106b.

19. The method of claim 6, wherein the miR-specific inhibitor comprises a polynucleic acid molecule that is essentially complementary to miR-106a.

20. The method of claim 6, wherein the miR-specific inhibitor comprises a polynucleic acid molecule that is 100% complementary to miR-106a.

21. A method of up-regulating p21 in a mammalian cell comprising introducing into said mammalian cell an effective amount of a miR-specific inhibitor of at least one miR-106b family member into the mammalian cell.

22. The method of claim 21, wherein said mammalian cell is a cancer cell.

23. The method of claim 21, wherein the at least one miR-106b family member is selected from the group consisting of miR-106b, miR-106a, miR-20a, miR-20b, and miR-17-5p.

24. The method of claim 21, wherein the at least one miR-106b family member comprises miR-106b.

25. The method of claim 21, wherein the at least one miR-106b family member comprises miR-106a.

26. The method of claim 21, wherein the miR-specific inhibitor is selected from the group consisting of anti-miR and target mimics.

27. The method of claim 21, wherein the miR-specific inhibitor comprises a nucleotide sequence of least 6 consecutive nucleotides that are complementary to the positions 2-8 of the seed region of said miR-106b family member, and has at least 50% complementarity to the rest of said miR-106b family member sequence, and wherein the miR-specific inhibitor of at least one miR-106b family member retards the G1-to-S transition.

28. The method of claim 27, wherein said miR-specific inhibitor is chemically modified on at least one nucleotide.

29. The method of claim 28, wherein said chemical modification comprises LNA.

30. The method of claim 28, wherein said chemical modification comprises 2′-O-methyl.

31. The method of claim 24, wherein the miR-specific inhibitor comprises a polynucleic acid molecule that is essentially complementary to miR-106b.

32. The method of claim 24, wherein the miR-specific inhibitor comprises a polynucleic acid molecule that is 100% complementary to miR-106b.

33. A method of down-regulating p21 in a mammalian cell comprising introducing into said mammalian cell an effective amount of a miR-106b family member.

34. The method of claim 33, wherein the at least one miR-106b family member is selected from the group consisting of miR-106b, miR-106a, miR-20a, miR-20b, and miR-17-5p.

35. The method of claim 33, wherein the at least miR-106b family member comprises miR-106b.

36. A method of accelerating proliferation of a cell comprising introducing an effective amount of a small interfering nucleic acid (siNA) into the cell, wherein said siNA comprises a guide strand contiguous nucleotide sequence of at least 18 nucleotides, wherein said guide strand comprises a seed region consisting of nucleotide positions 1 to 10, wherein position 1 represents the 5′ end of said guide strand and wherein said seed region comprises a nucleotide sequence of at least 6 contiguous nucleotides at positions 2 to 8 that are identical to SEQ ID NO:3.

37. The method of claim 36, wherein said siNA further comprises a non-nucleotide moiety.

38. The method of claim 36, wherein the guide strand and the passenger strand are stabilized against nucleolytic degradation.

39. The method of claim 36, wherein said siNA further comprises at least one chemically modified nucleotide or non-nucleotide at the 5′ end and/or 3′ end of the guide strand and the 3′ end of the passenger strand.

40. The method of claim 36, wherein said siNA comprises SEQ ID NO: 1.

41. The method of claim 36, wherein said siNA comprises SEQ ID NO: 4.

42. The method of claim 36, wherein said siNA comprises SEQ ID NO: 6.

43. The method of claim 36, wherein said siNA comprises SEQ ID NO: 8.

44. The method of claim 36, wherein said siNA comprises SEQ ID NO: 10.

45. A method for determining the cell cycle progression phenotype of a cell sample obtained from a subject, comprising:
a) measuring the level of at least one miR-106b family member in the cell sample; and
b) comparing the level of at least one miR-106b family member with a cell cycle progression reference value, wherein a level greater than the cell cycle progression reference value is indicative of an accelerated cell cycle progression in the cell sample.

46. The method of claim 45, wherein said at least one miR-106b family member is selected from the group consisting of miR-106b, miR-106a, miR-20a, miR-20b, and miR-17-5p.

47. The method of claim 45, wherein said the at least miR-106b family member comprises miR-106b.
